(19) United States
(12) Patent Application Publication
(10) Pub. No.: US 2008/0008707 A1
(43) Pub. Date: Jan. 10, 2008

(54) Denatured collagen peptides and uses thereof

(75) Inventors: Bruce Freimark, Laguna Niguel, CA (US); Dennis Van Epps, Santa Barbara, CA (US); Derek Clark, Morgantown, WV (US)

Correspondence Address: WILSON SONSINI GOODRICH & ROSATI, 650 PAGE MILL ROAD, PALO ALTO, CA, 94304-1050, US

(73) Assignee: Cell-Matrix, Inc., Carlsbad, CA (US)

(21) Appl. No.: 11/820,004

(22) Filed: Jun. 14, 2007

Related U.S. Application Data
(60) Provisional application No. 60/813,724, filed on Jun. 14, 2006.

(51) Int. Cl. A61K 39/395; A61P 43/00; C07K 16/18; C07K 7/00

(52) U.S. Cl. 424/133.1; 424/138.1; 424/139.1; 530/350; 530/387.3; 530/387.9; 530/388.1; 530/389.1

(57) ABSTRACT

The invention provides peptide antagonists, such as synthetic collagen peptides. The invention provides antibody antagonists, or functional fragments thereof, that preferentially bind to denatured extracellular matrix components. It additionally provides methods for using the antagonists for inhibiting angiogenesis, tumor metastasis, and other tumor developmental processes, including cell migration, cell adhesion, cell proliferation, and tumor growth and for treating angiogenesis-dependent conditions or collagen-dependent conditions. The application also provides for use of the antagonists as a vaccine for inducing an immune response, immune focusing and induction of antibody responses.

### CROSS-REFERENCE

This application claims the benefit of U.S. Provisional Application No. 60/813,724, filed Jun. 14, 2006, which application is incorporated herein by reference in its entirety.

### FIELD OF THE INVENTION

The invention relates generally to the field of medicine, and relates specifically to compositions that act as antagonists of denatured or proteolyzed forms of collagen, and uses thereof.

### BACKGROUND OF THE INVENTION

Identification of proteins involved in tumor cell interactions with the proteolytically-remodeled ECM can provide novel therapeutic targets and treatment strategies for treating malignant tumors. While many studies have confirmed the importance of targeting specific secreted growth factors, proteases, cell surface adhesion receptors and intracellular regulatory molecules, the success of these approaches has been limited due in part to the genetic instability of tumor cells (Molife, et al., Crit. Rev. Oncol. Hematol. 2002, 44:81-102; Brown, et al., Melanoma 2001, 3:344-352; Soengas, et al., Oncogene 2003, 22:3138-3151; Masters, et al., Nat. Rev. Cancer 2003, 3:517-525). Metastasis, or the spread of malignant tumor cells from the primary tumor mass to distant sites, involves a complex series of interconnected events. Understanding the biochemical, molecular, and cellular processes that regulate tumor metastasis are of great importance to treating these tumors. The metastatic cascade is thought to be initiated by a series of biochemical and genetic alterations leading to changes in cell-cell interactions allowing disassociation of cells from the primary tumor mass. These events are followed by local invasion and migration through the proteolytically-remodeled extracellular matrix (ECM) to allow access of the tumor cells to the host circulation. In order to establish secondary metastatic deposits, the malignant cells evade the host immune surveillance, arrest in the microvasculature and extravasate out of the circulation. Finally, circulating tumor cells can adhere to the ECM in a new location, proliferate, and recruit new blood vessels by induction of angiogenesis, thereby forming secondary metastatic foci (Liotta, et al., Cell 1991, 64:327-336; Wyckoff, et al., Cancer Res. 2000, 60:2504-2511; Kurschat, et al., Clin. Exp. Dermatol. 2000, 25:482-489; Pantel, et al., Nat. Rev. Cancer 2004, 4:448-456; Hynes, et al., Cell 2003, 113:821-823; Bashyam, M. D., Cancer 2002, 94:1821-1829). Therefore, identifying new functional targets within the non-cellular compartment provides a promising clinical strategy.

The ECM is an interconnected molecular network that not only provides mechanical support for cells and tissues, but also regulates biochemical and cellular processes such as adhesion, migration, gene expression and differentiation. Extracellular matrix components include, e.g., collagen, fibronectin, osteopontin, laminin, fibrinogen, elastin, thrombospondin, tenascin and vitronectin.

Cryptic sites within ECM components, including those within collagen recognized by the HUI77 antibody, regulate angiogenesis and endothelial cell behavior (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210). These functional cryptic sites were shown to be highly expressed within the ECM of malignant tumors and within the subendothelial basement membrane of tumor-associated blood vessels, and this exposure was found to be involved in the regulation of angiogenesis in vivo (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210, and U.S. Ser. No. 09/478,977, now U.S. Pub. No. 2003/0113331; the disclosure of each of which is incorporated herein by reference in its entirety).

Cryptic sites in the ECM component, laminin, have also been described, e.g., in U.S. Publication No. 2004/224896 A1 (the disclosure of which is incorporated herein by reference in its entirety), and WO 2004/087734.

There are potentially important cryptic epitopes in other ECM proteins, e.g., fibronectin (Hocking, et al., J. Cell. Biol. 2002, 158:175-184), fibrinogen (Medved et al., Ann. N.Y. Acad. Sci. 2001, 936:185-204) and osteopontin (Yamamoto, et al., J. Clin. Invest. (2003) 12:181-188).

Angiogenesis is the physiological process by which new blood vessels develop from pre-existing vessels (Varner, et al., Cell Adh. Commun. 1995, 3:367-374; Blood, et al., Biochim. Biophys. Acta. 1990, 1032:89-118; Weidner, et al., J. Natl. Cancer Inst. 1992, 84:1875-1887). Angiogenesis has been suggested to play a role in both normal and pathological processes. For example, angiogenic processes are involved in the development of the vascular systems of animal organs and tissues. These processes are also involved in transitory phases of angiogenesis, for example during the menstrual cycle, in pregnancy, and in wound healing. On the other hand, a number of diseases are known to be associated with deregulated angiogenesis.

In certain pathological conditions, angiogenesis is stimulated as a means to provide adequate blood and nutrient supply to the cells within affected tissue. Many of these pathological conditions involve aberrant cell proliferation and/or regulation. Therefore, inhibition of angiogenesis is a potentially useful approach to treating diseases that are characterized by new blood vessel development. For example, angiogenesis is involved in pathologic conditions including: ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis. Angiogenesis is also involved in cancer-associated disorders, including, for example, solid tumors, tumor metastases, angiofibromas, Kaposi's sarcoma, benign tumors such as hemangiomas, acoustic neuromas, neurofibromas, as well as other tumors which require neovascularization to support tumor growth. Other angiogenesis-dependent conditions include, for example, hereditary diseases such as Osler-Weber Rendu disease and hemorrhagic teleangiectasia; myocardial angiogenesis; plaque neovascularization; hemophiliac joints and wound granulation. Progression of tumors such as melanoma, from benign to metastatic disease, correlates with an increase in angiogenesis as well as an increase in expression of specific cell adhesion receptors including integrins (Srivastava, et al., Am. J. Pathol. 1988, 133:419-423; Koth, et al., N. Engl. J. Med. 1991, 325:171-182). Thus, angiogenesis likely plays a critical role in melanoma progression.

Examples of normal physiological processes involving angiogenesis include embryo implantation, embryogenesis and development, and wound healing. It is conceivable that angiogenesis can also be altered to beneficially influence normal physiological processes. Furthermore, studies have indicated that adipose tissue growth is dependent on angiogenesis, likely due to the need for recruitment of new blood vessels. Delivery of an angiogenesis inhibitor to mice was found to reduce diet-induced obesity, the most common type of obesity in humans (Brakenhielm, et al., Circ. Res. 2004, 94(12):1579-88). This finding suggests utility for angiogenesis inhibitors in addressing obesity and certain related conditions. Therefore, the inhibition of angiogenesis potentially can be applied in normal angiogenic responses where a prophylactic or therapeutic need or benefit exists.

In certain pathological conditions, interactions of integrins on cells, such as tumor cells, with an ECM component is as a means to develop tumors (or loci of tumor cells). Many of these pathological conditions involve aberrant cell proliferation or regulation. Progression of tumors such as melanoma, from benign to metastatic disease, correlates with an increase in expression of specific cell adhesion receptors including integrins (Srivastava, et al., Am. J. Pathol. 1988, 133:419-423; Koth, et al., N. Engl. J. Med. 1991, 325:171-182). Therefore, inhibition of interactions with cell-surface integrins is a potentially useful approach to treating diseases that are characterized by aberrant cell proliferation or regulation.

The ECM, in a vastly simplified fashion, can be characterized as being composed of two general compartments. Embracing this two-compartment concept, the ECM can be divided into the interstitial ECM and the basal lamina or basement membrane. The basement membrane is a specialized form of ECM that separates both epithelia and endothelia from their underlying mesenchyme (Timpl, R. (1989) European Journal of Biochemistry 180(3), 487-502; Timpl, R., and Brown, J. C. (1996) Bioessays 18(2), 123-32; Yurchenco, P. D., and Schittny, J. C. (1990) FASEB Journal 4(6), 1577-90; Schittny, J. C., and Yurchenco, P. D. (1989) Current Opinion in Cell Biology 1(5), 983-8). Components of the basement membrane include, for example, laminin, Type IV collagen, enactin/nidogen, SPARC and perlecan, as well as other proteoglycans. These components exhibit a complex pattern of molecular interconnections and supramolecular assemblies that are organized into a mesh-like network.

The mesh-like network of the basement membrane is connected to the underlying interstitial matrix by a series of anchoring fibers including collagen-VII and fibrillin. Some of the well-characterized components include a variety of genetically distinct forms of collagen, such as collagen-I, II, III and V. In addition, a number of non-collagenous glycoproteins including fibronectin, fibrinogen/fibrin, thrombospondin, and vitronectin also help compose the interstitial matrix (Adechi, E., Hopkinson, I., and Hayashi, T. (1997) International Review of Cytology 173, 73-156; Mosher, D. F., Sottile, J., Wu, C.; and McDonald, J. A. (1992) Curr. Opin. Cell Biol. 4, 810-818). Finally, a number of proteoglycans also contribute to the complex architecture of the interstitial matrix. The networks of proteins that make up the ECM in conjunction with integrins function cooperatively to regulate new blood vessel development.

Historically, the ECM was thought to provide mechanical and structural support to cells and tissues. However, following the development of new molecular, cellular and biochemical techniques, this limited view of the ECM has expanded dramatically. In fact, the ECM can be defined in broad terms as a complex interconnected network of fibrous proteins, proteoglycans and structural glycoproteins that provide both mechanical and biochemical regulatory functions to cells and tissues. In angiogenesis, the regulatory information contained within the three dimensional structure of the ECM must be recognized and transferred to recipient cells capable of forming new blood vessels. To this end, integrin-mediated ligation of ECM components has been shown to activate distinct signal transduction pathways which, in turn, may regulate neovascularization.

An important group of molecules that mediate cellular interactions with the ECM include the integrin family of cell adhesion receptors. Integrins are a family of heterodimeric cell surface proteins composed of non-covalently associated a and β chains (Jin, et al., Br. J. Cancer. 2004, 90:561-565; Bershadsky, et al., Annu. Rev. Cell Dev. Biol. 2003, 19:677-695, and; Parise, et al., Semin. Cancer Biol. 2003, 10:407-414). Integrins not only facilitate physical interactions with the ECM but also play critical roles in bi-directional signaling between the ECM and cells. In this regard, αvβ3 is one of the most well-studied integrins thought to play a critical role in invasive cellular processes such as angiogenesis and tumor invasion (Jin, et al., Br. J. Cancer. 2004, 90:561-565; Bershadsky, et al., Ann. Rev. Cell Dev. Biol. 2003, 19:677-695; Parise, et al., Semin. Cancer Biol. 2003, 10:407-414). In fact, expression of αvβ3 in endothelial cells regulates cell survival and apoptosis by a mechanism that likely depends on p53 (Stromblad, et al., J. Clin. Invest. 1996, 98:426-433; Stromblad, et al., J. Biol. Chem. 2002, 277:13371-13374; Lewis, et al., Proc. Natl. Acad. Sci. USA. 2002, 99:3627-3632). Therefore, αvβ3 ligation might suppress p53 activity. Furthermore, antagonists of αvβ3 failed to inhibit retinal neovascularization in p53 null mice. Stromblad, et al., J. Clin. Invest. 1996, 98:426-433; Stromblad, et al., J. Biol. Chem. 2002, 277:13371-13374).

Studies have indicated that integrins play a critical role in angiogenesis since antagonists directed to integrins inhibit angiogenesis and tumor growth in multiple models (Brooks, et al., Science 1994, 264:569-571; Brooks, et al., Cell, 1994, 79:1157-1164; Brooks, et al., J. Clin. Invest. 1995, 96:1815-1822). However in recent studies, mice lacking expression of αvβ3 exhibited enhanced growth of transplanted tumors (Taverna, et al., Proc. Natl. Acad. Sci. USA. 2001, 101:763-768). Interestingly, αvβ3 and αvβ5 may regulate angiogenesis induced by distinct growth factors by mechanisms dependent on differential phosphorylation of Raf (Hood, et al., J. Cell Biol. 2003, 162:933-943; Alavi, et al., Science 2003, 301:204-206). Studies have also provided evidence that integrins can regulate signaling cascades in both the unligated and ligated states (Stupack, et al., J. Cell Biol. 2001, 155:459-470). Furthermore, studies suggest that unligated αvβ3 may lead to induction of apoptosis by a mechanism involving recruitment of caspase-8 (Stupack, et al., J. Cell Biol. 2001, 155:459-470). Thus, the ability of an integrin to either interact or not with distinct ligands may differentially impact invasive cellular behavior.

Proteolytic activity plays a crucial role in controlling angiogenesis by releasing matrix-sequestered growth factors as well as remodeling ECM proteins. ECM remodeling of the matrix can alter the three-dimensional structure of ECM proteins such as collagen and laminin, thereby exposing cryptic regulatory sites that are recognized by integrins (Xu, et al., J. Cell Biol. 2001, 154:1069-1079; Hangai, et al., Am. J. Pathol. 2002, 161:1429-1437; Xu, et al., Hydridoma 2000, 19:375-385).

The physiological importance of cellular interactions with these cryptic sites has been suggested, since function-blocking monoclonal antibodies (Mabs, mAbs) directed to the cryptic collagen binding site block angiogenesis and tumor growth in a number of animal models (Xu, et al., J. Cell Biol. 2001, 154:1069-1079; Hangai, et al., Am. J. Pathol. 2002, 161:1429-1437; Xu, et al., Hydridoma 2000, 19:375-385). Manipulating the interactions between integrins and ECM components could provide a productive strategy for identifying methods to treat tumor development processes, including, but not limited to, tumor metastasis, tumor growth, angiogenesis, cell migration, cell adhesion and cell proliferation. However, the genes regulated in response to interactions involving integrin receptors and cryptic ECM components have not been previously characterized, and relatively little is known concerning the potential role of these interactions in tumor development processes.

Other proteins appear to be involved in integrin signaling such as, for example, Insulin Growth Factor Binding Proteins (IGFBPs). IGFBPs are a family of secreted proteins that function to regulate IGF-signaling by binding to IGFs, thereby disrupting IGF receptor binding and subsequent signaling (Pollak, et al., Nat. Rev. Cancer 2004, 4:505-518; Mohan, et al., J. Endocrinol. 2002, 175:19-31; LeRoith, et al., Cancer Lett. 2003, 195:127-137). Specific IGFBPs may directly bind to integrin receptors, thereby modulating their function independently from IGFs (McCaig, et al., J. Cell Sci. 2002, 115:4293-4303; Schutt, et al., J. Mol. Endocrinol. 2004, 32:859-868; Furstenberger, et al., Lancet. 2002, 3:298-302). IGFBPs may regulate cellular adhesion, migration and tumor growth by both IGF-dependent and -independent mechanisms (McCaig, et al., J. Cell Sci. 2002, 115:4293-4303; Schutt, et al., J. Mol. Endocrinol. 2004, 32:859-868; Furstenberger, et al., Lancet. 2002, 3:298-302). However, understanding of the regulation of these cellular processes by integrin-receptor binding of IGFBPs and the exact role of IGFBPs in these processes, have not been established.

Molecular alterations that occur in both tumor and stromal cells are thought to potentiate angiogenesis in part by modifying expression and bioavailability of angiogenic growth factors as well as altering expression of matrix-degrading proteases. Collectively, these and other molecular changes help to create a microenvironment conducive to new blood vessel growth, one factor that contributes to metastasis and tumor growth. There is evidence for the importance of numerous molecular regulators that contribute to new blood vessel growth, including matrix-degrading proteases such as matrix metalloprotease 9 (MMP-9), angiogenesis inhibitors such as thrombospondin-1 (TSP-1) and angiogenic growth factors such as VEGF (see, e.g., Yu, et al., Proc. Natl. Acad. Sci. USA 1999, 96:14517-14522; Dameron, et al., Science 1994, 265:1582-1584). These molecular regulators, the proteins that in turn regulate them and any of a number of other molecules potentially affect angiogenesis and metastasis. However, the exact mechanisms of the regulation of these and related processes, including the genes and gene expression patterns involved, have not been determined.

Further, the protein Id-1 has been reported to repress TSP-1 expression and regulate angiogenesis in vivo (Volpert, et al., Cancer Cell 2002, 2(6):473-83). P53, a tumor-suppressor protein, has also been reported to play an important role in controlling expression of proteins known to regulate angiogenesis, including vascular endothelial growth factor (VEGF) and TSP-1 (Yu, et al., Proc. Natl. Acad. Sci. USA 1999, 96:14517-14522 and Dameron, et al., Science 1994, 265:1582-1584). The p53 status of tumors is believed to impact the efficacy of anti-angiogenic, chemotherapeutic and radiation therapy for the treatment of malignant tumors (Yu, et al., Science 2002, 295:1526-1528; Martin, et al., Cancer Res. 1999, 59:1391-1399; Fridman, et al., Oncogene 2003, 22:9030-9040; and Gudkov, et al., Nat. Rev. Cancer 2003, 3:117-128).

### SUMMARY OF THE INVENTION

The present application is directed to antagonists that preferentially bind to a binding site on denatured (dn) collagen. Identified herein are specific binding sites (epitopes) and uses thereof. Also encompassed within the present application are compositions of the antagonists, polypeptides, methods of inducing immune responses, vaccines (immune compositions), pharmaceutical packages, kits, methods of blocking ligand binding to a denatured or non-denatured ECM component, methods of inhibiting angiogenesis, methods of preventing or treating a cancer or a metastasis, methods of preventing or treating a cell proliferative disorder, methods of treating diabetic retinopathy, mascular degeneration or neovascular glaucoma, methods of monitoring treatment and methods of imaging or diagnosis.

Provided herein is an antagonist that preferentially binds to a binding site on a denatured collagen, wherein said binding site consists essentially of, or consists of, a polypeptide having an amino acid sequence set forth as SEQ ID NO: 81 (GPPGPP) wherein one or more proline residues is hydroxyproline.

Provided herein is an antagonist that preferentially binds to a binding site on a denatured collagen, wherein said binding site consists essentially of an isolated polypeptide having an amino acid sequence set forth as PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline.

Provided herein is an antagonist that preferentially binds to a binding site on a denatured collagen, wherein said binding site consists of an isolated polypeptide having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline.

The antagonist can be an antibody or functional fragment thereof, including, but not limited to, a polyclonal antibody, a monoclonal antibody, a chimeric antibody, a humanized antibody, a human antibody, a labeled antibody, a Fab, a F(ab)2, a F(ab′)2, a scFv and a genetically engineered antibody.

The antagonist can inhibit angiogenesis, or prevent, inhibit, or treat an angiogenesis-dependent disorder, a cell proliferative disorder or a collagen-dependent disorder.

Provided herein is a composition of a pharmaceutically acceptable carrier/excipient and any of the antagonists provided herein. The composition can further include a therapeutic moiety, an imaging or diagnostic moiety. When the composition is to be administered to a patient, the composition can be substantially free of pyrogens. Compositions include, for example, pharmaceutical compositions for the therapeutic and diagnostic methods described herein.

Provided herein is a method of inducing an immune response in a patient by administering to the patient a pharmaceutical composition of an antagonist (e.g., antibody or fragment thereof), wherein the pharmaceutical composition contains an anti-human antibody or fragment thereof that induces an effective host immune response against the binding site of said antibody or fragment thereof. The antibody or functional fragment thereof of the composition blocks binding of an integrin to an extracellular matrix component. The extracellular matrix component can be, for example, cryptic collagen epitopes, cryptic laminin epitopes, fibronectin, vitronectin, fibrinogen, thrombospondin, osteopontin, tenascin, or vWF. An integrin can be, for example, an integrin listed in Table 1.

Provided herein is a method of blocking binding of a ligand to an extracellular matrix component comprising administering the pharmaceutical composition of an antagonist (e.g., antibody or fragment thereof), to a subject in need thereof. The extracellular matrix component can be, for example, cryptic collagen epitopes, cryptic laminin epitopes, fibronectin, vitronectin, fibrinogen, thrombospondin, osteopontin, tenascin, or vWF. The ligand can be, for example, an integrin listed in Table 1.

Provided herein is an antagonist that preferentially binds to a ligand of a denatured type IV collagen, wherein binding of the antagonist to the ligand blocks binding of the ligand to denatured type IV collagen, and said antagonist is a peptide.

In one embodiment, the isolated peptide consists essentially of, or consists of, an amino acid sequence set forth as SEQ ID NO: 81 (GPPGPP) wherein one or more proline residues is hydroxyproline.

In one embodiment, the isolated peptide consists essentially of an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline.

In one embodiment, the isolated peptide consists of an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline.

Any of the peptides, variants or peptidomimetics thereof can be linear or cyclic, and can bind to an integrin. Binding of said peptide, or variant or peptidomimetic thereof, to an integrin inhibits or prevents said integrin from binding to an extracellular matrix component. An integrin is, for example, one of those listed in Table 1. Binding of said peptide, or variant or peptidomimetic thereof, to an integrin, can inhibit angiogenesis, or prevent, inhibit or treat an angiogenesis-dependent disorder, a cell proliferative disorder or a collagen-dependent disorder.

The composition can include a pharmaceutically acceptable carrier/excipient. In one embodiment, the composition is a pharmaceutically acceptable salt of the peptide, or variant or peptidomimetic thereof. The composition can further include a therapeutic moiety, an imaging or diagnostic moiety. In some cases, it will be preferable that the composition is lyophilized to increase the shelf life of the composition in storage. When the composition is to be administered to a patient, the composition can be substantially free of pyrogens. Compositions include, for example, pharmaceutical compositions for administration to patients for the therapeutic and diagnostic methods described herein.

Provided herein is a vaccine containing one or more of: a) a peptide as provided herein; b) an antagonist as provided herein; c) an antibody as provided herein; and d) an anti-human antibody (Ab1) that binds to a peptide as provided herein. In one embodiment, the composition is an immunogenic composition. Provided herein is a method of vaccinating a subject by administering any of the vaccines provided herein.

Provided herein is pharmaceutical package containing any of the compositions and/or vaccines provided herein. Optionally, the pharmaceutical package can further include a label for inhibiting an angiogenesis-dependent disease or disorder, such as, for example, ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and hemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

Provided herein is a kit containing any of the compositions and/or vaccines provided herein. The kit can further comprising instructions for use. In one embodiment, the kit can further include other components for diagnostic assays (e.g., ELISA or ELISPOT). In another embodiment, the kit can further include accessories needed for administering the compositions to a patient (e.g., syringes).

Provided herein is a method for inducing a host immune response in a patient against the composition of an denatured extracellular matrix component (e.g., denatured collagen), by administering to the patient a pharmaceutical composition, where the pharmaceutical composition comprises an anti-human antibody or fragment thereof that induces an effective host immune response against the binding site of said antibody or fragment thereof.

The host immune response can be a humoral immune response or a cell-mediated immune response. If the immune response is a humoral immune response, it can be a protective antibody response that inhibits angiogenesis, an angiogenesis-dependent disease or an angiogenesis-dependent disorder. The angiogenesis-dependent disease or disorder can be, for example, ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and hemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like. In one embodiment, the protective antibody response inhibits binding of an integrin to an extracellular matrix component (e.g., denatured collagen).

Provided herein is a method of blocking ligand (e.g., integrin) binding to a denatured or non-denatured ECM component by administering a pharmaceutical composition provided herein to a subject. Blocking ligand binding to the ECM component can inhibit angiogenesis, and inhibition of angiogenesis can treat a cancer or alleviate symptoms associated with a cancer. Blocking can be mediated by administering an antagonist that binds to the ligand or by administering an antagonist that binds the denatured or non-denatured ECM component. Exemplary ECM component include, but are not limited to, denatured collagen fragments such as those described herein.

Provided herein is a method of inhibiting angiogenesis or an angiogenesis-dependent disease or disorder in a subject by administering a pharmaceutical composition provided herein to a patient. The angiogenesis-dependent disease or disorder can be any of the following: ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and hemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like. In one embodiment, inhibiting angiogenesis or an angiogenesis-dependent disease or disorder alleviates symptoms associated with the disease or disorder. In another embodiment, inhibiting angiogenesis or an angiogenesis-dependent disease or disorder results in decreased tumor size, prevention of tumor progression, decreased cell proliferation, decreased numbers of cells, inhibiting increased cell proliferation, inhibiting increases in numbers of cells, increased apoptosis, or decreased survival, of at least a portion of the cells comprising the angiogenesis. Inhibiting in angiogenesis can result in decreased tumor size or prevents tumor progression. The method can further include surgical removal of the cancer, and/or administration of an anti-cancer agent to a patient suffering from cancer.

Provided herein is a method of preventing or treating a cancer or metastasis in a subject by administering a pharmaceutical composition provided herein. In one embodiment, administration of the pharmaceutical composition prolongs life of the subject. A cancer/tumor to be treated includes, but is not limited to, a solid tumor, a metastasis, a cancer, a melanoma, a skin cancer, a breast cancer, a hemangioma or angiofibroma and the like cancer. Exemplary solid tumors are of a tissue or organ selected from among skin, melanoma, lung, pancreas, breast, ovary, colon, rectum, stomach, thyroid, laryngeal, ovarian, prostate, colorectal, head, neck, eye, mouth, throat, esophagus, chest, bone, testicular, lymphoid, marrow, bone, sarcoma, renal, sweat gland, liver, kidney, brain, and the like tissues.

The method can further include surgical removal of the cancer and/or administration of an anti-cancer agent. The anti-cancer agent can be administered prior to, concomitant with, or subsequent to, administration of the pharmaceutical composition. The anti-cancer agent can be administered within a week before the pharmaceutical composition, within a week after the pharmaceutical composition, or the anti-cancer agent can be administered on the same day as the pharmaceutical composition. If the anti-cancer agent is administered on the same day as the pharmaceutical composition, administration can be concomitant.

Provided herein is a method of preventing or treating a cancer or metastasis in a subject by administering a pharmaceutical composition provided herein to the subject. Administration of the pharmaceutical composition can prolong life of the subject. The cancer can by any of a solid tumor, a metastasis, a cancer, a melanoma, a skin cancer, a breast cancer, a hemangioma or angiofibroma and the like cancer. The method can further include surgical removal of the cancer. Alternatively, the method can further (or in addition) include administration of an anti-cancer agent or treatment. The anti-cancer agent or treatment can be administered prior to, concomitant with, or subsequent to, administration of the pharmaceutical composition. In one embodiment, the anti-cancer agent or treatment is administered within a week before the pharmaceutical composition. In another embodiment, the anti-cancer agent or treatment is administered within a week after the pharmaceutical composition.

Provided herein is a method for preventing or treating a cancer or a metastasis by surgical removal of the cancer/tumor and concurrent administration of an anti-cancer agent or treatment and a pharmaceutical composition provided herein to a subject.

Provided herein is a method of inhibiting angiogenesis by contacting a cell or tissue with a therapeutically effective amount of any of the antagonists or peptides as described herein sufficient to inhibit angiogenesis.

Provided herein is a method, comprising contacting a cell with any of the antagonists or peptides as described herein, wherein contacting inhibits binding of an integrin to an extracellular matrix component. The cell can be a cultured cell or can be present in a subject.

Provided herein is a method of preventing or treating a cell proliferative disorder by administering to a subject having or at risk of having a cell proliferative disorder an effective amount of a pharmaceutical composition provided herein effective to treat the cell proliferative disorder. In one embodiment, at least a part of the cells comprising the cell proliferative disorder is located in blood, breast, lung, thyroid, head or neck, brain, lymph, gastrointestinal tract, nasopharynx, genito-urinary tract, bladder, kidney, pancreas, liver, bone, muscle, skin, ovary, colon, rectum, stomach, thyroid, laryngeal, ovary, prostate, mouth, throat, esophagus, chest, bone, testicular, lymphoid, marrow, bone, sarcoma, renal, sweat gland, liver or the like tissues. The cell proliferative disorder can be, for example a benign or malignant solid or non-solid tumor and the tumor can be metastatic or non-metastatic. Exemplary solid tumors include, but are not limited to, a sarcoma or carcinoma. Exemplary non-solid tumors include, but are not limited to, a hematopoietic cancer (e.g., a myeloma, lymphoma or leukemia). The treatment can results in improving the subject's condition and can be assessed by determining if one or more of the following factors has occurred: decreased cell proliferation, decreased numbers of cells, inhibiting increased cell proliferation, inhibiting increases in numbers of cells, increased apoptosis, or decreased survival, of at least a portion of the cells comprising the cell proliferative disorder. Optionally, the method can further include administering an anti-cancer agent or treatment to the subject.

Provided herein is a method for treating diabetic retinopathy, macular degeneration or neovascular glaucoma in a patient by administering to the patient a therapeutically effective amount of a pharmaceutical composition provided herein.

Provided herein is a method of monitoring the efficacy of one or more of any of the methods provided herein.

In the methods provided herein, the subject can be a human or a non-human subject. Pharmaceutical compositions and the anti-cancer agent or treatments provided herein can be administered once or multiple times depending on the health of the patient, the progression of the disease or condition, and the efficacy of the treatment. Adjustments to therapy and treatments can be made throughout the course of treatment.

Pharmaceutical compositions can be administered locally, regionally or systemically, such as, for example, administration subcutaneously, intradermally, intravenously, intra-arterially, intraperitoneally, and intramuscularly.

Provided herein is a method of imaging or diagnosing angiogenesis or an angiogenic-dependent disease or disorder comprising contacting a composition of an antagonist or peptide as described herein with a sample. The sample can be, for example, blood, serum, platelets, biopsy fluid, spinal tap fluid, meninges, and urine. Imaging or diagnosis method can occur in an in vitro assay. Alternatively, when contacting is by administration of the composition to a patient, the angiogenesis or angiogenic-dependent disease or disorder is imaged or diagnosed in vivo.

Provided herein is a method for assessing proteomics profile of a sample, comprising a) dividing a plurality of antibodies into an unlabelled portion and a labeled portion; b) attaching the unlabelled antibodies on a solid surface to form an array of unlabelled antibodies on said solid surface; c) contacting said array of unlabelled antibodies formed in b) with a biosample to retain antigens contained in said biosample that specifically bind to said unlabelled antibodies; and d) detecting said retained antigens by contacting said retained antigens with said labeled antibodies, wherein proteomics profile of said biosample is assessed.

Provided herein is a method for assessing proteomics profile of a biosample, comprising a) dividing a plurality of peptides or peptidomimetics into an unlabeled portion and a labeled portion; b) attaching the unlabelled peptides or peptidomimetics on a solid surface to form an array of unlabeled peptides or peptidomimetics on said solid surface; c) contacting said array of unlabeled peptides or peptidomimetics formed in b) with a biosample to retain antigens contained in said biosample that specifically bind to said unlabeled peptides or peptidomimetics; and d) detecting said retained antigens by contacting said retained antigens with said labeled peptides or peptidomimetics, wherein proteomics profile of said biosample is assessed.

Provided herein is a method of selecting one or more cells by contacting a sample containing cells with an antagonist that preferentially binds to a binding site on a denatured collagen.

Provided herein is the use of the compounds of the present invention for use in generating synthetic or bioartificial tissues, such as skin.

One embodiment of the present invention contemplates the use of any of the compositions of the present invention to make a medicament for treating a disorder of the present invention. Medicaments can be formulated based on the physical characteristics of the patient/subject needing treatment, and can be formulated in single or multiple formulations based on the stage of the cancerous tissue. Medicaments of the present invention can be packaged in a suitable pharmaceutical package with appropriate labels for the distribution to hospitals and clinics wherein the label is for the indication of treating a disorder as described herein in a subject. Medicaments can be packaged as a single or multiple units. Instructions for the dosage and administration of the pharmaceutical compositions of the present invention can be included with the pharmaceutical packages.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 D93 was shown to compete with HUI77 for binding to human denatured-collagen IV. A series of 12 dilutions of HUI77 was prepared at twice the final concentration in 1% BSA/PBS, mixed with an equal volume of D93 (Research Lot No. 2, 14.4 mg/mL) diluted to 50 (circle; •), 10 (triangle; ▴), or 5 (square; ▪) μg/mL or 1% BSA/PBS alone (diamond; ♦) and pre-incubated at room temperature for 15 minutes prior to adding to dn-collagen coated wells. Data shown is the average of duplicate readings for each point at each concentration.

FIG. 2 Time Course for Digesting Collagen IV with Trypsin. Human collagen IV (5 mg/mL) was digested for 18 hours at 37° C. with trypsin at 100 μg/mL (lane marked 1), 10 μg/mL (lane marked 2), 1 μg/mL (lane marked 3), 0.1 μg/mL (lane marked 4), 0.01 μg/mL (lane marked 5), or no trypsin (lane marked C). Size markers shown are broad range (black: M) and polypeptide (red: M2) from BioRad. All lanes contain 5 μL of markers or digested collagen per lane. PAGE was performed using a pre-cast 16.5% acrylamide gel run in Tris/tricine buffer.

FIG. 3 Trypsin Digestion of Collagen IV for Western Blot Analysis and Protein Sequencing. Trypsin-digested (Tryp) or non-treated (NT) collagen IV was subjected to SDS/PAGE in reducing conditions (Panel A), and the gel was stained with Coomassie Blue. Trypsin-digested collagen IV was subjected to SDS/PAGE in reducing conditions, and peptides were electro-blotted to PVDF membrane for Western blot analysis or total protein staining with Coomassie Blue (Panel B). The same protein markers (M, Invitrogen Benchmark) were used for both Panels A and B. Arrows indicate collagen fragments 23, 35, and 57 kDa.

FIG. 4 All of the amino terminal peptide sequences identified in the 23, 35, and 57 kDa collagen fragment samples shown in Table 10 were located on the alpha I chain of collagen IV (SEQ ID NO: 44). The locations of the peptide sequences shown in Table 10 in the protein sequence of the alpha I chain of collagen IV are shown. The location of the N-terminal amino acid sequences of the 23 (Bold and italics; residues 176-190 and 1064-1079), 35 (Underline; residues 1239-1253) and 57 (Bold and underline; residues 598-608) kDa peptides reactive to D93 by Western blot analysis are highlighted in colors. Underlined sequences indicate the primary sequence identified by Edman degradation sequencing.

FIG. 5 Location of Peptide Array Corresponding to Sequences in Human Collagen IV Alpha I Chain (SEQ ID NO: 44). Small peptide sequences located in close proximity to peptides were identified by Edman sequencing was performed by screening a synthetic peptide array consisting of the C-terminal 1/3 of human collagen IV alpha 1 chain. This region was chosen based on the location of the N-terminal sequences of the 23 (secondary sequence) and 35 (primary) kDa peptides. The region selected for synthesis of the peptide array is shown. Underlined sequences indicate the region of collagen IV alpha I chain that corresponded to the peptide array. The peptide array consisted of 16-mers with an N-terminal biotin with a 10 amino acid overlap. Each proline position consisted of a 50% proline/hydroxyproline mixture. The sequence of peptide No. 40 is provided in bold font (i.e., residues 1327-1352).

FIG. 6 Binding of a Synthetic Collagen IV Peptide by D93. Panel A is a diagram showing the general format of the ELISA for screening the biotinylated peptide array. Panel B shows binding of peptide by D93 (black bars) and inhibition of binding by pre-incubation with heat-denatured-collagen IV (open bars). Data is shown as the average of duplicate samples.

FIG. 7 Depicts the location of GPPG (SEQ ID NO: 35) and GPPGPPG (SEQ ID NO: 81) sequences in Human Alpha 1 Collagen IV Chain (SEQ ID NO: 44). Amino acid residues 1-27 represent the signal peptide; amino acid residues 28-172 represent the pro-collagen domain; amino acid residues 173-1390 represent the triple-helical collagen domain; and amino acid residues 1391-1669 represent the C-terminal pro-collagen or NC-1/NC-2 domain. Single GPPG sequences (single underlined) and multimerized GPPG sequences (SEQ ID NO: 35; double underline) are primarily in the triple-helical region. Annotations for human alpha 1 collagen IV obtained from accession number P02462 at the “pir.georgetown.edu” website.

FIG. 8 Homology of Alpha 1 Collagen IV Chains from Different Species. Chicken, human and mouse αI (IV) (SEQ ID NOS: 63, 44 and 58, respectively) chains were aligned using the Clustal W program. Asterisks indicate 100% homology between sequences while semi-colon and period represent strong or weak homologies, respectively. Referenced locations of hydroxyprolines residues of the human αI (IV) are shown with an underlined “P”. An “O” above the sequence alignment indicates a potential hydroxyproline residue for at least two species. The boxed sequences are residues P1337-Y1352 which were identified by using D93 to screen a synthetic peptide array. Annotations for al collagen IV chains were obtained from the “pir.georgetown.edu” website using accession numbers P02462 (human), P02463 (mouse), and Q9I9K3 (chicken).

FIG. 9 Binding Kinetics of D93 to Denatured Collagen IV and Synthetic Collagen Peptide. Upper panels show binding of D93 to sensors coated with low (A; left panel), medium (B; middle panel), and high (C; right panel) densities of denatured collagen IV. Lower panels show binding of D93 to sensors coated with low (D; left panel), medium (E; middle panel), and high (F; right panel) densities of biotinylated synthetic collagen peptide were coated onto streptavidin-coated sensor chips. Binding kinetics were obtained from six concentrations of D93 tested in triplicate. Calculations were performed using the CLAMP program. The low density peptide surface data using a simple 1:1 interaction model, and the medium and higher density surfaces using an avidity model as was used to fit D93 binding to denatured collagen.

FIG. 10 An alignment of amino acid sequences of mouse, chicken and human collagen IV alpha I chain (SEQ ID NOS: 58, 63 and 44, respectively). The presence of the sequence GPPGPP (SEQ ID NO: 81) is observed in all three species and in multiple locations. Underlined sequences indicate the region of collagen IV alpha I chain that corresponded to the peptide array. The peptide array consisted of 16-mers with an N-terminal biotin with a 10 amino acid overlap. Each proline position consisted of a 50% proline/hydroxyproline mixture.

FIG. 11 An alignment of amino acid sequences of mouse and human collagen IV alpha 2 chain (SEQ ID NOS: 59 and 45, respectively). The presence of the sequence GPPGPP (SEQ ID NO: 81) is observed in all three species and in multiple locations.

FIG. 12 Location of GPPG (SEQ ID NO: 35) Amino Acid Sequences in the Human Collagen Type I Alpha I Chain (SEQ ID NO: 40). Amino acid residues 1-20 represent the signal peptide; amino acid residues 21-178 represent the pro-collagen domain; amino acid residues 179-1192 represent the triple-helical collagen domain; and amino acid residues 1193-1464 represent the C-terminal pro-collagen or NC-1/NC-2 domain. Underlined sequences are specific peptide sequences with potential to bind D93/HUI77. Annotations for human alpha 1 collagen I obtained from the website “pir.uniprot.org/cgi-bin/upEntry?id=P02452.”

FIG. 13 Location of GPPGPP (SEQ ID NO: 81) Amino Acid Sequences in the Human Collagen Type I Alpha I Chain (SEQ ID NO: 40). Amino acid residues 1-20 represent the signal peptide; amino acid residues 21-178 represent the pro-collagen domain; amino acid residues 179-1192 represent the triple-helical collagen domain; and amino acid residues 1193-1464 represent the C-terminal pro-collagen or NC-1/NC-2 domain. Underlined sequences are specific peptide sequences with potential to be bound by D93/HUI77. Annotations for human alpha 1 collagen I obtained from the website “pir.uniprot.org/cgi-bin/upEntry?id=P02452.”

FIG. 14 Location of GPPG (SEQ ID NO: 35) Amino Acid Sequences in the Human Collagen Type I Alpha 2 Chain (SEQ ID NO: 41). Amino acid residues 1-24 represent the signal peptide; amino acid residues 25-79 represent the pro-collagen domain; amino acid residues 80-1003 represent the triple-helical collagen domain; and amino acid residues 1004-1366 represent the C-terminal pro-collagen or NC-1/NC-2 domain. Underlined sequences are specific peptide sequences with potential to be bound by D93/HUI77. Annotations for human alpha 2 collagen I obtained from the website “pir.uniprot.org/cgi-bin/upEntry?id=P08123.”

FIG. 15 Location of GPPGPP (SEQ ID NO: 81) Amino Acid Sequences in the Human Collagen Type I Alpha 2 Chain (SEQ ID NO: 41). Amino acid residues 1-24 represent the signal peptide; amino acid residues 25-79 represent the pro-collagen domain; amino acid residues 80-1003 represent the triple-helical collagen domain; and amino acid residues 1004-1366 represent the C-terminal pro-collagen or NC-1/NC-2 domain. Underlined sequences are specific peptide sequences with potential to be bound by D93/HUI77. Annotations for human alpha 2 collagen I obtained from the website “pir.uniprot.org/cgi-bin/upEntry?id=P08123.”

FIG. 16 Location of GPPG (SEQ ID NO: 35) Amino Acid Sequences in the Human Collagen Type IV Alpha 1 Chain (SEQ ID NO: 44). Amino acid residues 1-27 represent the signal peptide; amino acid residues 28-172 represent the pro-collagen domain; amino acid residues 173-1390 represent the triple-helical collagen domain; and amino acid residues 1391-1669 represent the C-terminal pro-collagen or NC-1/NC-2 domain. Underlined sequences are specific peptide sequences with potential to be bound by D93/HUI77. Annotations for human alpha 1 collagen IV were obtained from the website “pir.uniprot.org/cgi-bin/upEntry?id=P02462.”

FIG. 17 Location of GPPGPP (SEQ ID NO: 81) Amino Acid Sequences in the Human Collagen Type IV Alpha 1 Chain (SEQ ID NO: 44). Amino acid residues 1-27 represent the signal peptide; amino acid residues 28-172 represent the pro-collagen domain; amino acid residues 173-1390 represent the triple-helical collagen domain; and amino acid residues 1391-1669 represent the C-terminal pro-collagen or NC-1/NC-2 domain. Underlined sequences are specific peptide sequences with potential to be bound by D93/HUI77. Annotations for human alpha 1 collagen IV obtained from the website “pir.uniprot.org/cgi-bin/upEntry?id=P02462.”

FIG. 18 Location of GPPG (SEQ ID NO: 35) Amino Acid Sequences in the Human Collagen Type IV Alpha 2 Chain (SEQ ID NO: 45). Amino acid residues 1-25 represent the signal peptide; amino acid residues 26-182 represent the pro-collagen domain; amino acid residues 183-1489 represent the triple-helical collagen domain; and amino acid residues 1490-1712 represent the C-terminal pro-collagen or NC-1/NC-2 domain. Underlined sequences are specific peptide sequences with potential to be bound by D93/HUI77. Annotations for human alpha 2 collagen IV obtained from the website “pir.uniprot.org/cgi-bin/upEntry?id=P08572.”

FIG. 19 Location of GPPGPP (SEQ ID NO: 81) Amino Acid Sequences in the Human Collagen Type IV Alpha 2 Chain (SEQ ID NO: 45). Amino acid residues 1-25 represent the signal peptide; amino acid residues 26-182 represent the pro-collagen domain; amino acid residues 183-1489 represent the triple-helical collagen domain; and amino acid residues 1490-1712 represent the C-terminal pro-collagen or NC-1/NC-2 domain. Underlined sequences are specific peptide sequences with potential to be bound by D93/HUI77. Annotations for human alpha 2 collagen IV obtained from the website “pir.uniprot.org/cgi-bin/upEntry?id=P08572.”

FIG. 20 Inhibition of Tumor Growth by Monoclonal Antibodies D93 in Orthotopic Human Breast Tumor Model. The results indicate tumor growth over time after treatments of 1, 10 or 100 μg of D93. The effect of D93 on tumor growth was statistically significant compared to control DP-28 (100 μg). The asterisk indicates that p<0.05 compared to PBS+HBSS or DP-28. Standard error bars were determined by calculating the standard deviation of the mean (STDEV) for each treatment group with the aid of the Excel software. The standard error of the mean (STERROR) was then calculated by dividing the standard deviation (STDEV) by the square root of the total number of animals in that treatment group (n): STERROR=[STDEV/√n].

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In describing the present invention, the following terms will be employed, and are intended to be defined as indicated below. Unless otherwise indicated, all terms used herein have the same ordinary meaning as they would to one skilled in the art of the present invention.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

### Definitions

By the terms “amino acid residue” and “peptide residue” is meant an amino acid or peptide molecule without the —OH of its carboxyl group. In general, the abbreviations used herein for designating the amino acids and the protective groups are based on recommendations of the IUPAC-IUB Commission on Biochemical Nomenclature (see Biochemistry (1972) 11: 1726-1732). For instance, Met, Ile, Leu, Ala and Gly represent “residues” of methionine, isoleucine, leucine, alanine and glycine, respectively. By the residue is meant a radical derived from the corresponding a-amino acid by eliminating the OH portion of the carboxyl group and the H-portion of the α-amino group. The term “amino acid side chain” is that part of an amino acid exclusive of the —CH—(NH₂)COOH portion, as defined by K. D. Kopple, “Peptides and Amino Acids,” W. A. Benjamin Inc., New York and Amsterdam, 1996, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H—(the side chain of glycine).

For the most part, the amino acids used in the application are those naturally occurring amino acids found in proteins, or the naturally occurring anabolic or catabolic products of such amino acids which contain amino and carboxyl groups. Particularly suitable amino acid side chains include side chains selected from those of the following amino acids: glycine (Gly; G), alanine (Ala; A), valine (Val; V), cysteine (Cys; C), leucine (Leu; L), isoleucine (Ile; I), serine (Ser; S), threonine (Thr; T), methionine (Met; M), glutamic acid (Glu; E), aspartic acid (Asp; D), glutamine (Gln; Q), asparagine (Asn; N), lysine (Lys; K), arginine (Arg; R), proline (Pro; P), histidine (His; H), phenylalanine (Phe; F), tyrosine (Tyr; Y), and tryptophan (Trp; W), and those amino acids and amino acid analogs which have been identified as constituents of peptidoglycan bacterial cell walls.

The term “amino acid residue” further includes analogs, derivatives and congeners of any specific amino acid referred to herein, as well as C-terminal or N-terminal protected amino acid derivatives (e.g., modified with an N-terminal or C-terminal protecting group). For example, the present invention contemplates the use of amino acid analogs wherein a side chain is lengthened or shorted while still providing a carboxyl, amino or other reactive precursor functional group for cyclization, as well as amino acid analogs having variant side chains with appropriate functional groups).

An “amino acid motif” is a sequence of amino acids, optionally a generic set of conserved amino acids, associated with a particular functional activity.

The term “binding” refers to a direct association between two molecules, due to, for example, covalent, electrostatic, hydrophobic, and ionic and/or hydrogen-bond interactions under physiological conditions, and including interactions such as salt bridges and water bridges and any other conventional binding means.

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic non-polar group, consisting of Val, Leu and Ile,

(vii) a slightly-polar group, consisting of Met and Cys,

(viii) a small-residue group, consisting of Ser, Thr, Asp, Asn, Gly, Ala, Glu, Gln and Pro,

(ix) an aliphatic group consisting of Val, Leu, Ile, Met and Cys, and

(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art as described above.

A “conserved residue” is an amino acid that is relatively invariant across a range of similar proteins. Often conserved residues will vary only by being replaced with a similar amino acid, as described above for “conservative amino acid substitution”.

The letter “x” or “xaa” as used in amino acid sequences herein is intended to indicate that any of the twenty standard amino acids may be placed at this position unless specifically noted otherwise. For the purposes of peptidomimetic design, an “x” or an “xaa” in an amino acid sequence may be replaced by a mimic of the amino acid present in the target sequence, or the amino acid may be replaced by a spacer of essentially any form that does not interfere with the activity of the peptidomimetic.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid (nucleotide, oligonucleotide) and amino acid (protein) sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST amino acid searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (see, www.ncbi.nlm.nih.gov).

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

As used herein, the terms “peptide,” “polypeptide” and “protein” are used interchangeably and refer to two or more amino acids covalently linked by an amide bond or non-amide equivalent. The peptides of the invention can be of any length. For example, the peptides can have from about 5 to 100 or more residues, such as, 5 to 12, 12 to 15, 15 to 18, 18 to 25, 25 to 50, 50 to 75, 75 to 100, 5 to 25, 5 to 50, 5 to 100, or more in length. The peptides of the invention include L- and D-isomers, and combinations of L- and D-isomers. The peptides can include modifications typically associated with post-translational processing of proteins, for example, cyclization (e.g., disulfide or amide bond), phosphorylation, glycosylation, carboxylation, ubiquitination, myristylation or lipidation. A polypeptide or peptide can be a naturally-occurring sequence of, for example, a denatured collagen, or a variant thereof. The presence of one or more of hydroxyproline residues in place of proline residues in the sequence of the polypeptide or peptide, while not required, may enhance the activity of the polypeptide or protein. Alternatively, the presence of one or more of hydroxyproline residues in place of proline residues in the sequence of the polypeptide or peptide, while not required, may enhance binding of an antibody to the polypeptide or protein.

A “peptidomimetic” includes any modified form of an amino acid chain, such as a phosphorylation, capping, fatty acid modification and including unnatural backbone and/or side chain structures. As described below, a peptidomimetic comprises the structural continuum between an amino acid chain and a non-peptide small molecule. Peptidomimetics generally retain a recognizable peptide-like polymer unit structure.

The phrase “protecting group” as used herein means substituents which protect the reactive functional group from undesirable chemical reactions. Examples of such protecting groups include esters of carboxylic acids and boronic acids, ethers of alcohols and acetals and ketals of aldehydes and ketones. For instance, the phrase “N-terminal protecting group” or “amino-protecting group” as used herein refers to various amino-protecting groups which can be employed to protect the N-terminus of an amino acid or peptide against undesirable reactions during synthetic procedures. Examples of suitable groups include acyl protecting groups such as to illustrate, formyl, dansyl, acetyl, benzoyl, trifluoroacetyl, succinyl and methoxysuccinyl; aromatic urethane protecting groups such as, for example, benzyloxycarbonyl (Cbz); and aliphatic urethane protecting groups such as t-butoxycarbonyl (Boc) or 9-Fluorenylmethoxycarbonyl (FMOC).

Peptides disclosed herein further include compounds having amino acid structural and functional analogues, for example, peptidomimetics having synthetic or non-natural amino acids or amino acid analogues, so long as the mimetic has one or more functions or activities. The compounds of the invention therefore include “mimetic” and “peptidomimetic” forms.

Invention peptides and peptidomimetics therefore include peptides and peptidomimetics having a sequence that is not identical to a sequence of peptides and peptidomimetics sequences set forth herein. In one embodiment, a peptide or peptidomimetic has a sequence having 50%, 60%, 70%, 75%, 80%, 85%, 90%, 95%, or more identity with a sequence set forth herein.

The compounds of the invention, including polypeptides, antibodies, peptides and peptidomimetics can be produced and isolated using any method known in the art. Peptides can be synthesized, whole or in part, using chemical methods known in the art (see, e.g., Caruthers (1980) Nucleic Acids Res. Symp. Ser. 215 223; Horn (1980) Nucleic Acids Res. Symp. Ser. 225 232; and Banga, A. K., Therapeutic Peptides and Proteins, Formulation, Processing and Delivery Systems (1995) Technomic Publishing Co., Lancaster, Pa.). Peptide synthesis can be performed using various solid-phase techniques (see, e.g., Roberge (1995) Science 269:202; Merrifield (1997) Methods Enzymol. 289:3 13) and automated synthesis may be achieved, e.g., using the ABI 431A Peptide Synthesizer (Perkin Elmer) in accordance with the manufacturer's instructions.

Individual synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Polypeptides, antibodies, peptides and peptide mimetics can also be synthesized using combinatorial methodologies. Techniques for generating peptide and peptidomimetic libraries are well known, and include, for example, multipin, tea bag, and split-couple-mix techniques (for example, al-Obeidi (1998) Mol. Biotechnol. 9:205 223; Hruby (1997) Curr. Opin. Chem. Biol. 1:114 119; Ostergaard (1997) Mol. Divers. 3:17 27; and Ostresh (1996) Methods Enzymol. 267:220 234). Modified peptides can be further produced by chemical modification methods (see, for example, Belousov (1997) Nucleic Acids Res. 25:3440 3444; Frenkel (1995) Free Radic. Biol. Med. 19:373 380; and Blommers (1994) Biochemistry 33:7886 7896).

As used herein, “salts” include pharmaceutically-acceptable salts, esters, hydrates, solvates or other derivatives of the compounds include any such salts, esters and other derivatives that may be prepared by those of skill in this art using known methods for such derivatization and that produce compounds that may be administered to animals or humans without substantial toxic effects and that either are pharmaceutically active or are prodrugs. Pharmaceutically-acceptable salts include, but are not limited to, salts of alkali metals and alkaline earth metals, including but not limited to sodium salts, potassium salts, lithium salts, calcium salts and magnesium salts; transition metal salts, such as zinc salts, copper salts and aluminum salts; polycationic counter ion salts, such as but not limited ammonium and substituted ammonium salts and organic amine salts, such as hydroxyalkylamines and alkylamines; salts of mineral acids, such as but not limited to hydrochlorides and sulfates, salts of organic acids, such as but not limited acetates, lactates, malates, tartrates, citrates, ascorbates, succinates, butyrate, valerate and fumarates. Also contemplated herein are the corresponding esters.

The term “pharmaceutically acceptable salts” refers to the relatively non-toxic, inorganic and organic addition salts of the peptides. These salts can be prepared in situ during the final isolation and purification of the peptides, or separately by reacting a purified peptide in its free base form with a suitable organic or inorganic acid, and isolating the salt thus formed. Representative salts include the hydrochloride, hydrobromic, sulfate, bisulfate, phosphate, nitrate, acetate, valerate, oleate, palmitate, stearate, laurate, benzoate, lactate, tosylate, citrate, maleate, furmarate, succinate, tartrate, napthylate, mesylate, glucoheptonate, lactobionate, and laurlysulphonate salts and the like. (See, for example, Berge et al. (1977) “Pharmaceutical Salts,” J. Pharm. Sci. 66: 1-19).

Antagonists

Antagonists provided herein include an ECM-component antagonist or an antagonist that competes with the binding of a cell expressing a natural ligand of an ECM component. For example, agents can be used to block binding of a cell expressing an integrin to an ECM component (e.g., denatured collagen) by interacting with the integrin or the ECM component.

In one-non-limiting example, antagonists of an integrin bind to the integrin expressed by a cell and interfere with functional interactions of the ligand with an ECM component such as, for example, denatured collagen. Alternatively, antagonists can bind to one or more amino acid sequences (binding site) in an ECM component (e.g., sequences within denatured collagen) and interfere with functional interactions of binding of a cell expressing an integrin to the ECM component.

As used herein, the term “antagonists” refers to molecules or compounds including, but not limited to, antibodies, functional fragments thereof (antigen binding fragments), peptides or variants thereof, peptidomimetics, oligonucleotides, and small molecule compounds. Such antagonists are described in, e.g., U.S. Pat. No. 6,500,924; U.S. Pat. No. 5,753,230; U.S. Publication No. 2004/0063790 A1; U.S. Pub. No. 2004/0258691; U.S. Publication No. 2004/0265317; U.S. Publication No. 2005/0002936; and U.S. Publication No. 2004/0176334 (the disclosures of each of which are incorporated herein by reference in their entirety) as well as in the present application.

Antagonists of the invention preferentially bind to a denatured collagen, but bind with substantially reduced affinity to the native form of the collagen. A “substantially reduced affinity” is an affinity of about 2-fold lower than that for the denatured collagen, about 5-fold lower, about 10-fold lower, and even greater than 10-fold lower. Likewise, “substantially less” indicates a difference of at least about a 2 fold difference when referring to relative affinities. Antagonists preferably bind one or more of the denatured collagens types. In one non-limiting example, an antagonist binds to one or more of denatured collagens type I-XXVII but binds with substantially reduced affinity and/or avidity to one or more of native collagens type I-XXVII. In another non-limiting example, an antagonist binds to denatured collagen type-I but binds with substantially reduced affinity and/or avidity to native collagen type-I. In yet another non-limiting example, an antagonist binds to denatured collagen type-IV but binds with substantially reduced affinity and/or avidity to native collagen type-IV.

Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

In one embodiment, peptides containing epitopes or binding sites recognized by an antagonist can be used themselves. In one embodiment, binding sites defined, for example, by the monoclonal antibody HUI77 are themselves used as anti-angiogenic compositions. In one non-limiting example, the sequence of amino acids (i.e., peptide) that is preferentially bound by an antibody antagonist, or a functional fragment thereof, can be used to block binding of a natural ligand or a cell expressing a natural ligand to an ECM component. For example, the sequence of amino acids recognized by an antibody such as HUI77 can be synthetically generated and used to block binding of an integrin or a cell expressing an integrin (e.g., tumor cell) to a denatured ECM component (e.g., collagen).

As used herein, “linker” refers to an unstructured polypeptide linker region between a label of the present invention and portions of an antagonist. The linker can facilitate enhanced flexibility, and/or reduce steric hindrance between any two fragments. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the 1CI and LexA proteins.

As used herein, a “label” refers to therapeutic and/or imaging/detectable moieties. Methods for conjugating or linking proteins, peptides, peptidomimetics, antibodies and fragments thereof are well known in the art. Associations between antagonists and labels include any means known in the art including, but not limited to, covalent and non-covalent interactions. In one non-limiting embodiment, the label can be a toxin, a radionuclide, an iron-related compound, a dye, an imaging reagent, a fluorescent label or a chemotherapeutic agent that would be toxic when delivered to a cancer cell. Alternatively, the label can be a detectable label, such as a radionuclide, iron-related compound, a dye, an imaging agent or a fluorescent agent for immunodetection of target antigens.

The term “consisting essentially of” as used in reference to a peptide including one or more designated amino acid sequences indicates that no more than 20 to 30 amino acids are added to the designated amino acid sequence(s) and, furthermore, that these additional amino acids do not substantially alter the function of the designated amino acid sequence(s).

“Isolated” (used interchangeably with “substantially pure”) when applied to polypeptides means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Preferably, the polypeptide is also separated from substances such as antibodies or gel matrices (polyacrylamide) which are used to purify it.

ECM Components

As used herein, an “ECM component” is a component of the non-cellular compartment. ECM components include, e.g., fibrin, fibrinogen, vitronectin, von Willebrand's factor, osteopontin, bone sialoprotein I, collagen, laminin, elastin, thrombospondin, tenascin, osteopontin, and fibronectin, as well as other proteins and molecules found in association with these ECM components or found in the same location as these ECM components (Gustafsson, E., et al., R. Exp. Cell Res. 2000, 261:52-68; Werb, Z., et al., Ann. N.Y. Acad. Sci. 1998, 857:110-118, and; Heissig, et al., Curr. Opin. Hematol. 2003, 10:136-141).

The methods of the invention are suitable for use with a number of collagen or denatured molecules, including those from any animal. In one embodiment collagens are human collagens. Collagens may also be from any mammal such as rat, mouse, pig, rabbit etc. or from a bird such as chicken. Collagen types are well known in the art (see, e.g., Olsen, B. R. (1995) Curr. Op. Cell. Biol. 5:720-727; Kucharz, E. J. The Collagens: Biochemistry and Pathophysiology. Springer-Verlag, Berlin, 1992; Kuhn, K. in Structure and Function of Collagen Types, eds. R. Mayne and R. E. Burgeson, Academic Press, Orlando). Human collagens are preferred collagens.

As used herein, a “collagen” refers to any collagen, including but not limited to, recombinantly produced polypeptide, synthetically produced polypeptide and collagen extracted from cells and tissues including, but not limited to, normal and abnormal (e.g., tumorigenic, transformed, metastatic, etc) cells and tissues. Collagen includes related polypeptides from different species including, but not limited to animals of human and non-human origin. Collagen includes collagen, allelic variant isoforms, synthetic molecules encoded by nucleic acids, protein isolated from tissue and cells, and modified forms thereof. Exemplary collagen polypeptides include, but are not limited to, wild-type collagen polypeptides and wild-type precursor collagen polypeptides that include a signal peptide, a polymorphic forms thereof. Collagen, as used herein can refer to one or more chains that make up a multimer-type protein such as a triple helical coiled polypeptide.

Collagen includes collagen polypeptides from any species, including human and non-human species. Exemplary collagen polypeptides of human origin include, for example, human collagen I α1 chain (SEQ ID NO: 40), human collagen I α2 chain (SEQ ID NO: 41), human collagen II α1 chain (SEQ ID NO: 42), human collagen III α1 chain (SEQ ID NO: 43), human collagen IV α1 chain (SEQ ID NO: 44), human collagen IV α2 chain (SEQ ID NO: 45), human collagen IV α3 chain (SEQ ID NO: 46), human collagen IV α5 chain (SEQ ID NO: 47), human collagen IX α2 chain (SEQ ID NO: 48), human collagen V α1 chain (SEQ ID NO: 49), human collagen VI α2 chain (SEQ ID NO: 50), human collagen VI α3 chain (SEQ ID NO: 51), human collagen XIV α1 chain (SEQ ID NO: 52); human collagen XVII α1 chain (SEQ ID NO: 53), human collagen XVIII α1 chain (SEQ ID NO: 54), human collagen XXI α1 chain (SEQ ID NO: 55), human collagen XXIV α1 chain (SEQ ID NO: 56), human collagen XXVII α1 chain (SEQ ID NO: 57), human collagen VI alpha 1 chain (SEQ ID NO: 91), human collagen VII alpha 1 chain (SEQ ID NO: 92) and human collagen XII alpha 1 chain (SEQ ID NO: 93).

Collagen polypeptides of non-human origin include, but are not limited to, bovine, ovine, rat, rabbit, horse, primates such as gorillas, chimpanzees and macaques, poultry such as chickens and turkeys, pig, dog, cat, rodents such as mice and rats, and avian collagen polypeptides. Exemplary collagen polypeptides of non-human origin include, for example, murine collagen IV α1 chain (SEQ ID NO: 58); murine collagen IV α2 chain (SEQ ID NO: 59); murine collagen IX α2 chain (SEQ ID NO: 60); sheep collagen IV α3 chain (SEQ ID NO: 61); equine collagen IX α2 chain (SEQ ID NO: 62); chicken collagen IV α1 chain (SEQ ID NO: 63); chicken collagen XII α1 chain (SEQ ID NO: 64); bovine collagen III α1 chain (SEQ ID NO: 65); bovine collagen IX α2 chain (SEQ ID NO: 66); bovine collagen VII α1 chain (SEQ ID NO: 67); bovine collagen XII α1 chain (SEQ ID NO: 68); bovine collagen XVII α1 chain (SEQ ID NO: 69); bovine collagen XVIII α1 chain (SEQ ID NO: 70); bovine collagen XXI α1 chain (SEQ ID NO: 71); canine collagen IV α3 chain (SEQ ID NO: 72); canine collagen IV α5 chain (SEQ ID NO: 73); canine collagen IV α1 chain (SEQ ID NO: 74); canine collagen VII α1 chain (SEQ ID NO: 75); canine collagen XII α1 chain (SEQ ID NO: 76); canine collagen XIV α1 chain (SEQ ID NO: 77); canine collagen XVII α1 chain (SEQ ID NO: 78); canine collagen XXI α1 chain (SEQ ID NO: 79) and canine collagen XXIV α1 chain (SEQ ID NO: 80).

A collagen polypeptide can be identified, for example, by the presence of the sequence GPPG (SEQ ID NO: 35) and/or GPPGPP (SEQ ID NO: 81) where, in some instances, one or more of the P (proline) residues can be hydroxyproline (Hyp). By aligning the sequences of collagen polypeptides, one skilled in the art can identify corresponding residues, using conserved and identical amino acid residues as guides. In other instances, corresponding regions can be identified. Repeated GPHyp sequences have been implicated in folding of the collagen triple helix, which is well studied for type I collagen (Brodsky, B. and Persikov, A. (2005) Adv. Protein Chem., 70: 301-339; and McLaughlin, S. H. and Bulleid, N.J. (1998) Matrix Biol., 16: 369-377). Collagen I α1 and α2 chains are initially synthesized as pro-collagen chains with the C-terminal globular pro-peptides functioning to correctly align the chains for heterotrimer formation. Initiation of the triple-helix, or nucleation, requires post-translational hydroxylation of Pro to Hyp residues while the chains are unfolded. A pentamer of the GPHyp sequence at the C terminus of the triple-helix domain of the α1 and α2 chains of type I collagen has been implicated in the nucleation of the triple helix (McLaughlin, S. H. and Bulleid, N.J. (1998) Matrix Biol., 16: 369-377; and Beuvich et al. (2000) Biochemistry, 39: 4299-4308). Following nucleation, the triple-helix conformation is propagated in a zipper-like mechanism from the C to N terminus.

Non-human collagen polypeptides includes collagen polypeptides, allelic variant isoforms, synthetic molecules prepared from nucleic acids, protein isolated from non-human tissue and cells, and modified forms thereof. As with human collagen, non-human collagen also include fragments or portions of collagen that are of sufficient length or include appropriate regions to be retain at least one activity of full-length mature polypeptide such as binding to an integrin on a cell (e.g., a tumor cell). Integrins to which a collagen polypeptide or peptide thereof can bind include, but are not limited to, α1β1, α2β1, α3β1, α4β1, α5β1, α6β1, α7β1, α8β1, α9β1, α10β1, α11β1, αvβ1, αvβ3, αvβ5, αvβ8, αvβ6, αvβ8 and α6β4. Activity can be any level of percentage of activity of the polypeptide, including but not limited to, 1% of the activity, 2%, 3%, 4%, 5%, 10%, 20%, 30%, 40%, 50%, 60%, 70%, 80%, 90%, 95% or more of functional activity compared to the full polypeptide. Assays to determine functionality or activity of forms of collagen are known to those of skill in the art. Assays include, for example, an ELISA assay, a radioimmunoassay, or any other conventional assay to identify binding of an antibody or functional fragment thereof to the collagen polypeptide or portion thereof or to identify binding of a cell expressing a cell-surface integrin to the collagen polypeptide or portion thereof; or inhibition of either type of interaction.

Denatured collagen refers to collagen that has been treated such that it no longer predominantly assumes the native triple helical form. Denaturation can be accomplished by heating the collagen. In one embodiment, collagen is denatured by heating to about 37° C.-100° C. for about 15-60 minutes. In another embodiment, collagen is denatured by exposing collagen to an enzyme such as, for example, trypsin. Denaturation can also be accomplished by treating the collagen with a chaotropic agent. Suitable chaotropic agents include, for example, guanidinium salts. Denaturation of a collagen can be monitored, for example, by spectroscopic changes in optical properties such as absorbance, circular dichroism or fluorescence of the protein, by nuclear magnetic resonance, by Raman spectroscopy, or by any other suitable technique. Denatured collagen refers to denatured full length collagens as well as to fragments of collagen. A fragment of collagen can be any collagen sequence shorter than a native collagen sequence. For fragments of collagen with substantial native structure, denaturation can be effected as for a native full-length collagen. Fragments also can be of a size such that they do not possess significant native structure or possess regions without significant native structure of the native triple helical form. Such fragments are denatured all or in part without requiring the use of heat or of a chaotropic agent. The term denatured collagen encompasses proteolyzed collagen. Proteolyzed collagen refers to a collagen that has been fragmented through the action of a proteolytic enzyme. In particular, proteolyzed collagen can be prepared by treating the collagen with a metalloproteinase, such as MMP-1, MMP-2 or MMP-9, or by treating the collagen with a cellular extract containing collagen degrading activity or is that which occurs naturally at sites of neovascularization in a tissue.

The methods of the invention contemplate the use of antagonists that inhibit binding of an integrin to an ECM component, including cryptic epitopes of ECM components, from any animal. For example, collagens may be from any mammal such as rat, mouse, pig, rabbit, or from a bird such as chicken, etc. Collagen types are well known in the art (see, e.g., Olsen, B. R. 1995, Curr. Op. Cell. Biol. 5:720-727; Kucharz, E. J. The Collagens: Biochemistry and Pathophysiology. Springer-Verlag, Berlin, 1992; Kuhn, K. in Structure and Function of Collagen Types, eds. R. Mayne and R. E. Burgeson, Academic Press, Orlando; U.S. Pub. No. 2003-0113331).

Laminins are a large family of extracellular matrix glycoproteins. Laminins have been shown to promote cell adhesion, cell growth, cell migration, cell differentiation, neurite growth, and to influence the metastatic behavior of tumor cells (U.S. Pat. No. 5,092,885). Laminin, of which there are at least ten isoforms, is a major component of basement membranes and has been shown to mediate cell-matrix attachment, gene expression, tyrosine phosphorylation of cellular proteins, and branching morphogenesis (Streuli, et al., J. Cell Biol. 1993, 129:591-603; Malinda and Kleinman, Int. J. Biochem. Cell Biol. 1996, 28:957-1959; Timpl and Brown, Matrix Biol. 1994, 14:275-281; Tryggvason, Curr. Op. Cell Biol. 1993 5:877-882; Stahl, et al., J. Cell Sci. 1997, 110:55-63). Laminin binds to type IV collagen, heparin, gangliosides, and cell surface receptors and promotes the adhesion and growth of various epithelial and tumor cells as well as neurite outgrowth. Laminin is thought to mediate cell-matrix interactions and to be a structural component of all basement membranes binding to collagen IV, heparin sulfate proteoglycan, and nidogen-entactin. The laminin molecule is composed of three polypeptide chains (α, β, and γ) assembled into a cross-shaped structure. Different α, β, and γ chains may be combined, which accounts for the large size of the laminin family (Jones, J. C. R. et al., Micr. Res. Tech. 2000, 51:211-213; Patarroyo, M. et al., Semin. Cancer Biol. 2002, 12:197-207).

Epitopes and Binding Sites

An “epitope” or “binding site” is an amino acid sequence or sequences that are “preferentially bound” or “specifically bound” by an antagonist of the invention. An epitope can be a linear peptide sequence (i.e., “continuous”) or can be composed of noncontiguous amino acid sequences (i.e., “conformational” or “discontinuous”). An antagonist can recognize one or more amino acid sequences; therefore, an epitope can define more than one distinct amino acid sequence target. The epitopes recognized by an antagonist can be determined by peptide mapping and sequence analysis techniques well known to one of skill in the art.

A “cryptic epitope of an ECM component” or a “cryptic binding site of an ECM component” is an epitope or binding site of an ECM component protein sequence that is not exposed or substantially protected from recognition within a native ECM component, but is capable of being recognized by an antagonist of a denatured or proteolyzed ECM component. Sequences that are not exposed, or are only partially exposed, in the native structure are potential cryptic epitopes. If an epitope is not exposed, or only partially exposed, then it is likely that it is buried within the interior of the molecule. The sequence of cryptic epitopes can be identified by determining the specificity of an antagonist. Candidate cryptic epitopes also can be identified, for example, by examining the three-dimensional structure of a native ECM component.

Angiogenesis and Diseases Potentially Treated by Inhibitors of Angiogenesis

As used herein, the terms “angiogenesis inhibitory,” “angiogenesis inhibiting” or “anti-angiogenic” include vasculogenesis, and are intended to mean effecting a decrease in the extent, amount, or rate of neovascularization. Effecting a decrease in the extent, amount, or rate of endothelial cell proliferation or migration in the tissue is a specific example of inhibiting angiogenesis.

The term “angiogenesis inhibitory composition” refers to a composition which inhibits angiogenesis-mediated processes such as endothelial cell migration, proliferation, tube formation and subsequently leading to the inhibition of the generation of new blood vessels from existing ones, and consequently affects angiogenesis-dependent conditions.

As used herein, the term “angiogenesis-dependent condition” is intended to mean a condition where the process of angiogenesis or vasculogenesis sustains or augments a pathological condition or beneficially influences normal physiological processes. Therefore, treatment of an angiogenesis-dependent condition in which angiogenesis sustains a pathological condition could result in mitigation of disease, while treatment of an angiogenesis-dependent condition in which angiogenesis beneficially influences normal physiological processes could result in, e.g., enhancement of a normal process.

Angiogenesis is the formation of new blood vessels from pre-existing capillaries or post-capillary venules. Vasculogenesis results from the formation of new blood vessels arising from angioblasts which are endothelial cell precursors. Both processes result in new blood vessel formation and are included in the meaning of the term angiogenesis-dependent conditions. The term “angiogenesis” as used herein is intended to include de novo formation of vessels such as that arising from vasculogenesis as well as those arising from branching and sprouting of existing vessels, capillaries and venules.

Examples of diseases in which angiogenesis plays a role in the maintenance or progression of the pathological state are listed herein. Other diseases are known to those skilled in the art and are similarly intended to be included within the meaning of “angiogenesis-dependent condition” and similar terms as used herein. For example, angiogenesis is involved in pathologic conditions including: ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis. Angiogenesis is also involved in cancer-associated disorders, including, for example, solid tumors, tumor metastases, angiofibromas, Kaposi's sarcoma, benign tumors such as hemangiomas, acoustic neuromas, neurofibromas, as well as other tumors which require neovascularization to support tumor growth. Other angiogenesis-dependent conditions include, for example, hereditary diseases such as Osler-Weber Rendu disease and hemorrhagic teleangiectasia; myocardial angiogenesis; plaque neovascularization; hemophiliac joints and wound granulation. Diseases and disorder to be treated using the compositions provided herein also include any collagen-dependent disease or disorder including, but not limited to, fibrocystic diseases (e.g., fibrosis and endometriosis), collagen based skin diseases (e.g., psoriasis, scleroderma, eczema), platelet based disorders associated with collagen (e.g., plaque formation, etc.), type II collagen arthritis, inflammatory diseases (e.g., restenosis, diabetic retinopathy, rheumatoid arthritis), opthalmic uses (e.g., macular degeneration), etc.

The present invention contemplates treatments comprising combinations of agents and/or other cancer therapies, said combinations having an effect on angiogenesis and being useful in methods for treating angiogenesis-dependent conditions. Treatments according to the invention can, for example, block interactions between two proteins by binding to one protein, binding to the other protein, or both. For example, agents can be used in combination to block integrin binding to an ECM component by interacting with the integrin, the ECM component, or both. Combinations of antagonists that bind to or interfere with interactions between different proteins, combinations of antagonists that bind to multiple parts of the same protein, and combinations of antagonists that bind to multiple proteins or protein binding sites are also contemplated. Further contemplated are treatments including bi- or multi-specific antagonists that interfere with more than one protein or protein-binding site.

Induction of Immune Responses

“Inducing a host immune response” means that a patient experiences alleviation or reduction of signs or symptoms of illness, and specifically includes, without limitation, prolongation of survival. In certain preferred embodiments of the methods according to the invention, a CD8+ IFN-γ producing T cell is activated to induce a cytotoxic T lymphocyte (CTL) immune response in the patient administered the antagonist. In certain embodiments of the methods according to the invention, a CD4+ IFN-γ producing T cell is activated to induce a helper T cell immune response in the patient administered with the composition. These activated CD4+ IFN-γ producing T cells (i.e., helper T cells) provide necessary immunological help (e.g. by release of cytokines) to induce and maintain not only CTL, but also a humoral immune response mediated by B cells. Thus, in certain embodiments of the methods according to the invention, a humoral response to the antigen is activated in the patient administered with the composition. In one aspect, an adjuvant may be added to the composition to increase an immune response. Adjuvants are well-known in the art.

Activation of a CD8+ and/or CD4+ T cells means causing T cells that have the ability to produce cytokines (e.g., IFN-γ) to actually produce one or more cytokine(s), or to increase their production of one or more cytokine(s). “Induction of CTL response” means causing potentially cytotoxic T lymphocytes to exhibit antigen specific cytotoxicity. “Antigen specific cytotoxicity” means cytotoxicity against a cell presenting an antigen that is associated with the antigen associated with the cancer that is greater than an antigen that is not associated with a cancer. “Cytotoxicity” refers to the ability of the cytotoxic T lymphocyte to kill a target cell. Such antigen-specific cytotoxicity can be at least 3-fold, at least 10-fold greater, at least 100-fold greater or more than cytotoxicity against a cell not presenting the antigen not associated with the cancer.

Cell Proliferative Disorders

As used herein, the terms “proliferative disorder” and “proliferative condition” mean any pathological or non-pathological physiological condition characterized by aberrant or undesirable proliferation of, for example, a cell, virus, bacteria, fungus, etc. The terms “cell proliferative disorder” and “cell proliferative condition” mean any pathological or non-pathological physiological condition characterized by aberrant or undesirable cell proliferation, as well as including conditions characterized by undesirable or unwanted cell proliferation or cell survival (e.g., due to deficient apoptosis), conditions characterized by deficient or aberrant or deficient apoptosis, as well as conditions characterized by aberrant or undesirable or unwanted cell survival. The term “differentiative disorder” means any pathological or non-pathological physiological condition characterized by aberrant or deficient differentiation.

Proliferative or differentiative disorders amenable to treatment include diseases and non-pathological physiological conditions, benign and neoplastic, characterized by abnormal or undesirable cell numbers, cell growth or cell survival. Such disorders or conditions may therefore constitute a disease state and include all types of cancerous growths or oncogenic processes, metastatic tissues or malignantly transformed cells, tissues, or organs, or may be non-pathologic, i.e., a deviation from normal but which is not typically associated with disease. A specific example of a non-pathologic condition that may be treated in accordance with the invention is tissue re-growth from wound repair that results in scarring.

Cells comprising the proliferative or differentiative disorder may be aggregated in a cell mass or be dispersed. The term “solid tumor” refers to neoplasias or metastases that typically aggregate together and form a mass. Particular examples include visceral tumors such as gastric or colon cancer, hepatomas, venal carcinomas, lung and brain tumors/cancers. A “non-solid tumor” refers to neoplasias of the hematopoietic system, such as lymphomas, myelomas and leukemias, or neoplasias that are diffuse in nature, as they do not typically form a solid mass. Particular examples of leukemias include acute and chronic lymphoblastic, myeloblastic and multiple myeloma.

Such disorders include neoplasms or cancers, which can affect virtually any cell or tissue type, e.g., carcinoma, sarcoma, melanoma, metastatic disorders or hematopoietic neoplastic disorders. A metastatic tumor can arise from a multitude of primary tumor types, including but not limited to breast, lung, thyroid, head and neck, brain, lymphoid, gastrointestinal (mouth, esophagus, stomach, small intestine, colon, rectum), genito-urinary tract (uterus, ovary, cervix, bladder, testicle, penis, prostate), kidney, pancreas, liver, bone, muscle, skin, etc.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the cervix, lung, prostate, breast, head and neck, colon, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas refer to malignant tumors of mesenchymal cell origin. Exemplary sarcomas include for example, lymphosarcoma, liposarcoma, osteosarcoma, and fibrosarcoma.

As used herein, the term “hematopoietic proliferative disorder” means a disease involving hyperplastic/neoplastic cells of hematopoietic origin, e.g., arising from myeloid, lymphoid or erythroid lineages, or precursor cells thereof. Typically, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

The methods of the invention are contemplated for use in treatment of a cell proliferative disorder such as, but not limited to, a cancer, a tumor or a metastasis. Thus, the methods of the invention are contemplated for use in treatment of a tumor tissue of a patient with a tumor, solid tumor, a metastasis, a cancer, a melanoma, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include, but are not limited to, tumors of the skin, melanoma, lung, pancreas, breast, ovary, colon, rectum, stomach, thyroid, laryngeal, ovarian, prostate, colorectal, head, neck, eye, mouth, throat, esophagus, chest, bone, testicular, lymphoid, marrow, bone, sarcoma, renal, sweat gland, liver, kidney, brain, and the like tissues. Further examples of cancers treated are glioblastomas.

A tissue to be treated is, for example, a retinal tissue of a patient with diabetic retinopathy, macular degeneration or neovascular glaucoma and the angiogenesis to be inhibited is retinal tissue angiogenesis where there is neovascularization of retinal tissue.

Thus, methods which inhibit angiogenesis in a diseased tissue ameliorate symptoms of the disease and, depending upon the disease, can contribute to cure of the disease. In one embodiment, the invention contemplates inhibition of angiogenesis in a tissue. The extent of angiogenesis in a tissue, and therefore the extent of inhibition achieved by the present methods, can be evaluated by a variety of methods, such as are described herein.

Any of a variety of tissues, or organs comprised of organized tissues, can support angiogenesis in disease conditions including skin, muscle, gut, connective tissue, joints, bones and the like tissue in which blood vessels can invade upon angiogenic stimuli. Thus, in one embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In the absence of neovascularization of tumor tissue, the tumor tissue does not obtain the required nutrients, slows in growth, ceases additional growth, regresses and ultimately becomes necrotic resulting in killing of the tumor. The present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because their formation requires vascularization of a primary tumor so that the metastatic cancer cells can exit the primary tumor and their establishment in a secondary site requires neovascularization to support growth of the metastases.

The invention also contemplates the practice of the method in conjunction with other therapies such as conventional chemotherapy directed against solid tumors and for control of establishment of metastases. The administration of an angiogenesis inhibitor is typically conducted during or after chemotherapy, although it is preferable to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

Treatments for use in combination with the invention compounds include any anti-cancer agent or treatment as disclosed herein or known in the art. For example, an anti-cell proliferative or anti-tumor treatment may comprise radiation treatment or surgical resection optionally in combination with drug treatment. The treatment may comprise administration of a chemical substance, such as a radioisotope, a drug, such as a chemotherapeutic agent, or genetic therapy, such as an anti-oncogene (e.g., Rb, DCC, p53, etc.), a dominant negative oncogene or an antisense to an oncogene. The compounds can be administered prior to, contemporaneously with or following other treatment protocols. For example, a candidate subject for anti-cell proliferative therapy (e.g., radiation therapy, chemotherapy, gene therapy, surgical resection, etc.) can be administered a compound described herein prior to initiating the anti-cell proliferative therapy. Thus, prophylactic treatment methods are provided.

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

The term “tumor cell antigen” is defined herein as an antigen that is present in higher quantities on a tumor cell or in body fluids than unrelated tumor cells, normal cells, or in normal body fluid. Tumor cell antigens also encompass fragments of ECM components that are shed as a result of breakdown of the ECM. The antigen presence may be tested by any number of assays known to those skilled in the art and include without limitation negative and/or positive selection with antibodies, such as an ELISA assay, a radioimmunoassay, or by Western Blot.

The terms “apoptosis” or “programmed cell death,” refers to the physiological process by which unwanted or useless cells are eliminated during development and other normal biological processes. Apoptosis is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy. Cells undergoing apoptosis show characteristic morphological and biochemical features. These features include chromatin aggregation, nuclear and cytoplasmic condensation, partition of cytoplasm and nucleus into membrane bound vesicles (apoptotic bodies), which contain ribosomes, morphologically intact mitochondria and nuclear material. In vivo, these apoptotic bodies are rapidly recognized and phagocytized by macrophages, dendritic cells or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis.” Apoptosis can be measured by methods known to those skilled in the art like DNA fragmentation, exposure of Annexin V, activation of caspases, release of cytochrome c, etc. A tumor cell that has been induced to die is termed herein as an “apoptotic tumor cell.”

“Apoptosis inducing agent” is defined herein to induce apoptosis/programmed cell death, and include, for example, irradiation, chemotherapeutic agents or receptor ligation agents, wherein cells, for example, tumor cells are induced to undergo programmed cell death.

Apoptosis can be tested using a standard Annexin V Apoptosis Assay: NIH:OVCAR-3 cells are grown in 6-well plates (NUNC) and irradiated or treated with an antagonist (or in combination with another anti-cancer drug) for 4-48 hours, washed and stained with Annexin V-FITC (BD-Pharmingen) for 1 hour. Cells are analyzed by flow cytometry (Becton-Dickinson, CellQuest), counterstained with Propidium Iodide and analyzed again in the flow cytometer.

Compositions

Each of the embodiments of the present invention can be used as a composition when combined with a pharmaceutically acceptable carrier or excipient. “Carrier” and “excipient” are used interchangeably herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable ratio.

“Pharmaceutically acceptable carrier” is defined herein as a carrier that is physiologically acceptable to the administered patient and that retains the therapeutic properties of the antibodies. Pharmaceutically-acceptable carriers and their formulations are and generally described in, for example, pharmaceutical Sciences (18th Edition, ed. A. Gennaro, Mack Publishing Co., Easton, Pa. 1990). One exemplary pharmaceutical carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antibodies from the administration site of one organ, or portion of the body, to another organ, or portion of the body. Each carrier must be “acceptable” in the sense of being compatible with the other ingredients of the formulation and not injurious to the patient. Nor should a pharmaceutically acceptable carrier alter the specific activity of the antibodies. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cacao butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl and ethyl laurate; agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutically acceptable formulation can incorporate about 1% to 99.9% of active ingredient (e.g., peptide or mimetic thereof or antibody or functional fragment thereof. The pharmaceutical compositions can be sterilized by conventional, well-known sterilization techniques, or can be sterile filtered.

The pharmaceutical formulations can be packaged in unit dosage form for ease of administration and uniformity of dosage. Unit dosage form as used herein refers to physically discrete unitary dosages for administration to the subject to be treated; each unit contains a predetermined quantity of compound that produces a desired effect in combination with a pharmaceutical carrier or excipient.

In one embodiment, the antagonists can be presented in lyophilized form. Methods of lyophilizing compounds are well-known in the art.

In another embodiment, the compounds and compositions of the present invention are substantially free of pyrogens. “Substantially free,” as used herein, refers to a level of pyrogens that will not cause an inflammatory reaction. Methods and kits for testing for toxic levels of pyrogens are well-known in the art and are commercially available.

Imaging/Diagnostic and Therapeutic Moieties

As used herein, “therapeutic moieties” are those such as “anti-cancer agents or treatments” refers to, but is not limited to, a chemotherapeutic agent, a nucleic acid damaging agent, a nucleic acid damaging treatment, an anticancer antibody, an anti-proliferative agent, or an anti-proliferative treatment to the subject.

As used herein, the terms “nucleic acid damaging treatment” and “nucleic acid damaging agent” mean any treatment regimen that directly or indirectly damages nucleic acid (e.g., DNA, cDNA, genomic DNA, mRNA, tRNA or rRNA). Specific examples of such agents include alkylating agents, nitrosoureas, anti-metabolites, plant alkaloids, plant extracts and radioisotopes. Examples of agents also include nucleic acid damaging drugs, for example, 5-fluorouracil (5-FU), capecitabine, S-1 (Tegafur, 5-chloro-2,4-dihydroxypyridine and oxonic acid), 5-ethynyluracil, arabinosyl cytosine (ara-C), 5-azacytidine (5-AC), 2′,2′-difluoro-2′-deoxycytidine (dFdC), purine antimetabolites (mercaptopurine, azathiopurine, thioguanine), gemcitabine hydrochloride (Gemzar), pentostatin, allopurinol, 2-fluoro-arabinosyl-adenine (2F-ara-A), hydroxyurea, sulfur mustard (bischloroetyhylsulfide), mechlorethamine, melphalan, chlorambucil, cyclophosphamide, ifosfamide, thiotepa, AZQ, mitomycin C, dianhydrogalactitol, dibromoducitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubibcin (Cerubicine), idarubicin (Idamycin) and epirubicin (Ellence), anthracyclin analogues such as mitoxantrone, actinomycin D, non intercalating topoisomerase inhibitors such as epipodophyllotoxins (etoposide=VP16, teniposide=VM-26), podophylotoxin, bleomycin (Bleo), pepleomycin, compounds that form adducts with nucleic acid including platinum derivatives (e.g., cisplatin (CDDP), trans analogue of cisplatin, carboplatin, iproplatin, tetraplatin and oxaliplatin), camptothecin, topotecan, irinotecan (CPT-11), and SN-38. Examples of nucleic acid damaging treatments include radiation (e.g., ultraviolet (UV), infrared (IR), or alpha-, beta- or gamma-radiation) and environmental shock (e.g., hyperthermia).

As used herein, the terms “anti-proliferative treatment” and “anti-proliferative agent” means any treatment regimen that directly or indirectly inhibits proliferation of a cell, virus, bacteria or other unicellular or multicellular organism regardless of whether or not the treatment or agent damages nucleic acid. Particular examples of anti-proliferative agents are anti-tumor and anti-viral drugs, which inhibit cell proliferation or virus proliferation or replication. Examples include, inter alia, cyclophosphamide, azathioprine, cyclosporin A, prednisolone, melphalan, chlorambucil, mechlorethamine, busulphan, methotrexate, 6-mercaptopurine, thioguanine, cytosine arabinoside, taxol, vinblastine, vincristine, doxorubicin, actinomycin D, mithramycin, carmustine, lomustine, semustine, streptozotocin, hydroxyurea, cisplatin, mitotane, procarbazine, dacarbazine and dibromomannitol. Anti proliferative agents that cause nucleic acid replication errors or inhibit nucleic acid replication are those such as nucleoside and nucleotide analogues (e.g., AZT or 5-AZC).

Chemotherapeutic agents contemplated by the present invention also include other chemotherapeutic drugs that are commercially available. Merely to illustrate, the chemotherapeutic can be an inhibitor of chromatin function, a inhibitor, a inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), a DNA repair inhibitor.

Chemotherapeutic agents may be categorized by their mechanism of action into, for example, the following groups: anti-metabolites/anti-cancer agents, such as pyrimidine analogs floxuridine, capecitabine, and cytarabine) and purine analogs, folate antagonists and related inhibitors antiproliferative/antimitotic agents including natural products such as vinca alkaloid (vinblastine, vincristine, and microtubule such as taxane (paclitaxel, docetaxel), vinblastin, nocodazole, epothilones and navelbine, epidipodophyllotoxins (etoposide, teniposide), DNA damaging agents (actinomycin, amsacrine, busulfan, carboplatin, chlorambucil, cisplatin, cyclophosphamide, cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); anti-platelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prenisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin. Preferred dosages of the chemotherapeutic agents are consistent with currently prescribed dosages.

As used herein, “radiation” refers to, for example, microwaves, ultraviolet (UV), infrared (IR), or alpha-, beta- or gamma-radiation. Radiation can be “focused” or locally delivered using conventional techniques to target radiation to the site of one or more tumors without radiating the entire body.

As used herein, an “environmental shock” refers to, for example, hyperthermia.

Patients

The invention contemplates treatment of patients including human and non-human patients. In a preferred embodiment, the patient is a human. The term patient as used in the present application refers to all different types of mammals including humans and the present invention is effective with respect to all such mammals. “Patient” and “subject” are used interchangeably herein. The present invention is effective in treating any mammalian species which have a disease associated with angiogenesis or which reduction of angiogenesis would result in treatment of a condition including tumor metastasis, tumor growth, cell adhesion, cell proliferation or cell migration. The present invention also has particular application to agricultural and domestic mammalian species including, but not limited to, horses, primates (e.g., gorillas, monkeys, chimpanzees), cows and bulls, dogs, cats, sheep, pigs, poultry (e.g., chickens, turkeys), rodents (e.g., mice, rats), or any other veterinary animal for which the invention has uses.

It will be appreciated that a “patient suffering from cancer” of the invention may not yet be symptomatic for the disease. For example, where the cancer is colon cancer (which is associated with the mutant K-ras protein), a patient with a mutant K-ras protein in some cells of the colon is a patient according to the invention even though that patient may not yet be symptomatic for colon cancer. “Associated with a mutant protein” means signs or symptoms of illness in a majority of patients are present when the mutant protein is present in the patient's body, but in which signs or symptoms of illness are absent when the mutant protein is absent from the patient's body. “Signs or symptoms of illness” are clinically recognized manifestations or indications of disease.

“Administering” is defined herein as a means providing the composition to the patient in a manner that results in the composition being inside the patient's body. Such an administration can be by any route including, without limitation, locally, regionally or systemically by subcutaneous, intradermal, intravenous, intra-arterial, intraperitoneal, or intramuscular administration (e.g., injection). “Concurrent administration” means administration within a relatively short time period from each other; such time period can be less than 2 weeks, less than 7 days, less than 1 day and could even be administered simultaneously.

“Contacting” is defined herein as a means of bringing a composition as provided herein in physical proximity with a cell, organ, tissue or fluid as described herein. Contacting encompasses systemic or local administration of any of the compositions provided herein and includes, without limitation, in vitro, in vivo and/or ex vivo procedures and methods. “Combining” and “contacting” are used interchangeably herein and are meant to be defined in the same way.

A physician or veterinarian having ordinary skill in the art can readily determine and prescribe the “effective amount” of a pharmaceutical composition. For example, the physician or veterinarian could start doses of the compounds of the invention employed in the pharmaceutical composition at levels lower than that required in order to achieve the desired therapeutic effect and gradually increase the dosage until the desired effect is achieved.

By “treating” a patient suffering from cancer it is meant that the patient's symptoms are partially or totally alleviated, or remain static following treatment according to the invention. A patient that has been treated can exhibit a partial or total alleviation of symptoms and/or tumor load. This is intended to encompass prophylaxis, therapy and cure. In one non-limiting example, a patient suffering from a highly metastatic cancer (e.g., breast cancer) is treated where additional metastasis either do not occur, or are reduced in number as compared to a patient who does not receive treatment. In another non-limiting example, a patient is treated where the patient's solid cancer either becomes reduced in size or does not increase in size as compared to a patient who does not receive treatment. In yet another non-limiting example, the number of cancer cells (e.g., leukemia cells) in a treated patient either does not increase or is reduced as compared to the number of cancer cells in a patient who does not receive treatment. Improvement can also be defined, for example, as decreased cell proliferation, decreased numbers of cells, inhibiting increased cell proliferation, inhibiting increases in numbers of cells, increased apoptosis, or decreased survival, of at least a portion of the cells comprising a cell proliferative disorder

A “therapeutically effective amount” is defined herein an effective amount of composition for producing some desired therapeutic effect by inducing tumor-specific killing of tumor cells in a patient and thereby blocking the biological consequences of that pathway in the treated cells eliminating the tumor cell or preventing it from proliferating, at a reasonable ratio applicable to any medical treatment.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

The term “sample” is defined herein includes, but is not limited to, blood, serum, blood product, biopsy tissue, biopsy fluid, serum, spinal tap fluid, meninges, platelets, and any other type of fluid or tissue that can be extracted from a patient.

Assays

The invention also provides assay methods for identifying candidate denatured collagen antagonists for use according to the present methods. In these assay methods candidate antagonists are evaluated for their ability to bind both denatured collagen and native collagen, and furthermore can be evaluated for their potency in inhibiting angiogenesis in a tissue.

ELISA

The assay determines/measures binding of antagonists to denatured or native collagens in solid phase by ELISA. The assay can also measure binding of antagonists to cells expressing an integrin that binds to denatured or native collagens in the solid phase by ELISA. The assay is useful with a variety of types of collagens, for example, the assay can be used with any of the chains of collagens types (e.g., I-XX) as well as for other extracellular matrix components.

The assay also can be used to identify compounds which exhibit specificity for denatured but not native forms of collagen. The specificity assay is conducted by running parallel ELISAs where a potential antagonist is screened concurrently in separate assay chambers for the ability to bind denatured and native collagens.

Antagonists of denatured collagen can also be identified by their ability to compete for binding with an antagonist of the invention. For example, putative antagonists can be screened by monitoring their effect on the affinity of a known antagonist, such as HUI77, in a binding assay, such as ELISA. Such antagonists likely have the same specificity as HUI77, and recognize the same cryptic epitope. Putative antagonists selected by such a screening method can bind either to the collagen or to the antagonist. Antagonists can be selected from the putative antagonists by conventional binding assays to determine those that bind to the denatured collagen epitope but not to the known antagonist.

Antagonists can also be identified by their ability to bind to a solid matrix containing a denatured collagen. Such putative antagonists are collected after altering solution conditions, such as salt concentration, pH, temperature, etc. The putative antagonists are further identified by their ability to pass through, under appropriate solution conditions, a solid matrix to which a native collagen has been affixed.

Angiogenesis Assays

Antagonists of the invention also can be assayed for their ability to modulate angiogenesis in a tissue. Any suitable assay known to one of skill in the art can be used to monitor such effects. Several such techniques are described herein.

The second assay measures angiogenesis in the chick chorioallantoic membrane (CAM) and is referred to as the CAM assay. The CAM assay has been described in detail by others, and further has been used to measure both angiogenesis and neovascularization of tumor tissues. See Ausprunk et al., Am. J. Pathol., 79:597-618 (1975) and Ossonski et al., Cancer Res., 40:2300-2309 (1980). The CAM assay is a well recognized assay model for in vivo angiogenesis because neovascularization of whole tissue is occurring, and actual chick embryo blood vessels are growing into the CAM or into the tissue grown on the CAM.

As demonstrated herein, the CAM assay illustrates inhibition of neovascularization based on both the amount and extent of new vessel growth. Furthermore, it is easy to monitor the growth of any tissue transplanted upon the CAM, such as a tumor tissue.

Finally, the assay is particularly useful because there is an internal control for toxicity in the assay system. The chick embryo is exposed to any test reagent, and therefore the health of the embryo is an indication of toxicity.

A third assay measures angiogenesis is the in vivo rabbit eye model and is referred to as the rabbit eye assay. The rabbit eye assay has been described in detail by others, and further has been used to measure both angiogenesis and neovascularization in the presence of angiogenic inhibitors such as thalidomide. See D'Amato et al. (1994) Proc. Natl. Acad. Sci. 91:4082-4085.

The rabbit eye assay is a well recognized assay model for in vivo angiogenesis because the neovascularization process, exemplified by rabbit blood vessels growing from the rim of the cornea into the cornea, is easily visualized through the naturally transparent cornea of the eye. Additionally, both the extent and the amount of stimulation or inhibition of neovascularization or regression of neovascularization can easily be monitored over time.

Finally, the rabbit is exposed to any test reagent, and therefore the health of the rabbit is an indication of toxicity of the test reagent.

A fourth assay measures angiogenesis in the chimeric mouse:human mouse model and is referred to as the chimeric mouse assay. The assay has been described in detail by others, and further has been described herein to measure angiogenesis, neovascularization, and regression of tumor tissues. See Yan, et al. (1993) J. Clin. Invest. 91:986-996.

The chimeric mouse assay is a useful assay model for in vivo angiogenesis because the transplanted skin grafts closely resemble normal human skin histologically and neovascularization of whole tissue is occurring wherein actual human blood vessels are growing from the grafted human skin into the human tumor tissue on the surface of the grafted human skin. The origin of the neovascularization into the human graft can be demonstrated by immunohistochemical staining of the neovasculature with human-specific endothelial cell markers.

The chimeric mouse assay demonstrates regression of neovascularization based on both the amount and extent of regression of new vessel growth. Furthermore, it is easy to monitor effects on the growth of any tissue transplanted upon the grafted skin, such as a tumor tissue. Finally, the assay is useful because there is an internal control for toxicity in the assay system. The chimeric mouse is exposed to any test reagent, and therefore the health of the mouse is an indication of toxicity.

### MODES OF CARRYING OUT THE INVENTION

It is to be understood that this invention is not limited to particular formulations or process parameters, as these may, of course, vary. It is also to be understood that the terminology used herein is for the purpose of describing particular embodiments of the invention only, and is not intended to be limiting. Further, it is understood that a number of methods and materials similar or equivalent to those described herein can be used in the practice of the present invention.

I. Antagonists

Provided herein are antagonists that inhibit angiogenesis, treat cancers, be used to monitor efficacy of treatment regimens and protocols and the like as described throughout the present application and known in the art. Such antagonists include, for example, antibodies or functional fragments thereof, polypeptides or variants thereof, peptides or variants thereof, peptidomimetics, small molecule inhibitors, oligonucleotides and the like. The peptides can be linear or cyclic. The invention also describes cell lines which produce the antibodies or functional fragments thereof, methods for producing the cell lines, and methods for producing the antibodies or functional fragments thereof. The invention also describes methods for synthesizing peptides.

The present invention describes, in one embodiment, antagonists that bind to an integrin expressed on a cell such as, but not limited to, α1β1, α2β1, α3β1, α4β1, α5β1, α6β1, α7β1, α8β1, α9β1, α10β1, α11β1, αvβ1, αvβ3, αvβ5, αvβ8, αvβ6, αvβ8 and/or α6β4. The present invention describes, in one embodiment, antagonists that bind to a denatured ECM component, but bind to the native ECM component with a substantially reduced avidity. Substantially reduced avidity includes, but is not limited to, 2-fold decreased avidity, 3-fold, 4-fold, 5-fold, 10-fold, 100-fold, etc. decreased avidity of binding to the native integrin. Such antagonists include, for example, antibodies or functional fragments thereof, polypeptides, peptides or variants thereof, peptidomimetics, small molecule inhibitors, oligonucleotides and the like. The invention also describes cell lines which produce the antibodies, methods for producing the cell lines, and methods for producing the antibodies.

The present invention describes, in one embodiment, peptide antagonists that can be used as vaccines. Peptide antagonists can be, for example, used to generate monoclonal antibodies, which monoclonal antibodies can be administered to a subject in need thereof. Peptide antagonists can also be administered to a subject and can generate an immune response to the peptide. Immune responses include generation of antibodies, initiation of T helper responses and/or initiation of a cytotoxic T lymphocyte reaction. In some instances, the peptide antagonist is linked to another moiety to render it more visible the immune system.

The present invention describes, in one embodiment, antagonists that preferentially bind to a site on a denatured ECM component (e.g, denatured collagen). Such antagonists include, for example, antibodies or functional fragments thereof, polypeptides, peptides or variants thereof, peptidomimetics, small molecule inhibitors, oligonucleotides and the like. The invention also describes cell lines which produce the antibodies, methods for producing the cell lines, and methods for producing the antibodies.

A. Antibodies

The present invention describes, in one embodiment, denatured collagen antagonists in the form of antibodies which bind to a denatured ECM component but bind to the native ECM component with a substantially reduced affinity and/or avidity. Antibody antagonists also can inhibit an angiogenesis-dependent disorder, a collagen-dependent disorder, or a cell-proliferative disorder. The invention also describes cell lines which produce the antibodies, methods for producing the cell lines, and methods for producing the monoclonal antibodies.

The term “antibody or antibody molecule” in the various grammatical forms is used herein as a collective noun that refers to a population of immunoglobulin molecules and/or immunologically active portions of immunoglobulin molecules, i.e., molecules that contain an antibody combining site or paratope.

As used herein, “immunoreactive” refers to binding agents, antibodies or fragments thereof that are specific to a sequence of amino acid residues (“binding site” or “epitope”), yet if are cross-reactive to other peptides/proteins, are not toxic at the levels at which they are formulated for administration to human use. The term “preferentially binds” means that the binding agent binds to the binding site with greater affinity than it binds unrelated amino acid sequences. Preferably such affinity is at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the binding agent for unrelated amino acid sequences. The terms “immunoreactive” and “preferentially binds” are used interchangeably herein.

The term “antibody” also includes molecules which have been engineered through the use of molecular biological technique to include only portions of the native molecule as long as those molecules have the ability to bind a particular antigen or sequence of amino acids with the required specificity. Such alternative antibody molecules include classically known portions of the antibody molecules, single chain antibodies, and single chain binding molecules.

An “antibody combining site” is that structural portion of an antibody molecule comprised of heavy and light chain variable and hypervariable regions that specifically binds antigen.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally between about 100-120 amino acids in length but is intended to reference only those amino acids outside of the CDRs. As used herein, the term “framework region” is intended to mean each domain of the framework that is separated by the CDRs.

Exemplary antibodies for use in the present invention are intact immunoglobulin molecules, substantially intact immunoglobulin molecules and those portions of an immunoglobulin molecule that contain the paratope, including those portions known in the art as Fab, Fab′, F(ab′)₂, F(v), single chain variable fragments (scFv), single chain binding polypeptides and others also referred to as antibody fragments.

In one exemplary embodiment, the invention contemplates a single chain binding polypeptide having a heavy chain variable region, a light chain variable region and, optionally, an immunoglobulin Fc region. Such a molecule is a single chain variable fragment optionally having effector function through the presence of the immunoglobulin Fc region. Methods of preparing single chain binding polypeptides are known in the art (e.g., US. Patent Application 2005/0238646).

In another preferred embodiment, the invention contemplates a truncated immunoglobulin molecule comprising a Fab fragment derived from a monoclonal antibody of this invention. The Fab fragment, lacking Fc receptor, is soluble, and affords therapeutic advantages in serum half life, and diagnostic advantages in modes of using the soluble Fab fragment. The preparation of a soluble Fab fragment is generally known in the immunological arts and can be accomplished by a variety of methods.

For example, Fab and F(ab′)₂ portions (fragments) of antibodies are prepared by the proteolytic reaction of papain and pepsin, respectively, on substantially intact antibodies by methods that are well known. See for example, U.S. Pat. No. 4,342,566 (Theofilopolous and Dixon). Fab′ antibody portions also are well known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact immunoglobulin molecules are preferred, and are utilized as illustrative herein.

The phrase “monoclonal antibody” in its various grammatical forms refers to a population of antibody molecules that contain only one species of antibody combining site capable of immunoreacting with a particular epitope. A monoclonal antibody may therefore contain an antibody molecule having a plurality of antibody combining sites, each immunospecific for a different epitope.

A monoclonal antibody is typically composed of antibodies produced by clones of a single cell called a hybridoma that secretes (produces) only one kind of antibody molecule.

The hybridoma cell is formed by fusing an antibody-producing cell and a myeloma or other self-perpetuating cell line. The preparation of such antibodies was first described by Kohler and Milstein, Nature 256:495-497 (1975), which description is incorporated by reference. Additional methods are described by Zola, Monoclonal Antibodies: A Manual of Techniques, CRC Press, Inc. (1987). The hybridoma supernatants so prepared can be screened for the presence of antibody molecules that immunoreact with denatured collagens. Alternatively, the hybridoma supernatants so prepared can be screened for the presence of antibody molecules that immunoreact with an integrin such as αvβ3.

Briefly, to form the hybridoma from which the monoclonal antibody composition is produced, a myeloma or other self-perpetuating cell line is fused with lymphocytes obtained from the spleen of a mammal hyperimmunized with a source of denatured collagen or integrin.

It is preferred that the myeloma cell line used to prepare a hybridoma be from the same species as the lymphocytes. Typically, a mouse of the strain 129 G1X+ is the preferred mammal. Suitable mouse myelomas for use in the present invention include the hypoxanthine-aminopterin-thymidine-sensitive (HAT) cell lines P3.times.63-Ag8.653, and Sp2/0-Ag14 that are available from the American Type Culture Collection, Rockville, Md., under the designations CRL 1580 and CRL 1581, respectively.

Splenocytes are typically fused with myeloma cells using polyethylene glycol (PEG) 1500. Fused hybrids are selected by their sensitivity to a selective growth medium, such as HAT (hypoxanthine aminopterin thymidine) medium. Hybridomas producing a monoclonal antibody of this invention are identified using the enzyme linked immunosorbent assay (ELISA) described in the Examples.

A monoclonal antibody of the present invention also can be produced by initiating a monoclonal hybridoma culture comprising a nutrient medium containing a hybridoma that secretes antibody molecules of the appropriate specificity. The culture is maintained under conditions and for a time period sufficient for the hybridoma to secrete the antibody molecules into the medium. The antibody-containing medium is then collected. The antibody molecules can then be further isolated by well known techniques.

Media useful for the preparation of these compositions are both well known in the art and commercially available and include synthetic culture media, inbred mice and the like. An exemplary synthetic medium is Dulbecco's minimal essential medium (DMEM; Dulbecco et al., Virol. 8:396, 1959) supplemented with 4.5 g/L glucose, 20 nM glutamine, and 20% fetal calf serum. One exemplary inbred mouse strain is the BALB/c.

Other methods of producing a monoclonal antibody, a hybridoma cell, or a hybridoma cell culture also are well known. See, for example, the method of isolating monoclonal antibodies from an immunological repertoire as described by Sastry et al. (1989) Proc. Natl. Acad. Sci. USA, 86:5728-5732; and Huse et al. (1989) Science, 246:1275-1281.

Also contemplated by this invention is the hybridoma cell and cultures containing hybridoma cells that produce monoclonal antibodies of this invention. Particularly preferred is a hybridoma cell line that secretes monoclonal antibody mAb HU177.

It also is possible to determine, without undue experimentation, if a monoclonal antibody has an equivalent specificity (immunoreaction characteristics) as a monoclonal antibody of this invention by ascertaining whether the former prevents the latter from binding to a preselected target molecule. If the monoclonal antibody being tested competes with the monoclonal antibody of the invention, as shown by a decrease in binding by the monoclonal antibody of the invention in standard competition assays for binding to the target molecule when present in the solid phase, then it is likely that the two monoclonal antibodies bind to the same, or a closely related, epitope or binding site.

An additional way to determine whether a monoclonal antibody has the specificity of a monoclonal antibody of the invention is to determine the amino acid residue sequence of the CDR regions of the antibodies in question. Antibody molecules having identical, or functionally equivalent, amino acid residue sequences in their CDR regions have the same binding specificity. Methods for sequencing polypeptides are well known in the art. This does not suggest that antibodies with distinct CDR regions cannot bind to the same epitope or binding site.

The immunospecificity of an antibody, its target molecule binding capacity, and the attendant affinity the antibody exhibits for the epitope, are defined by the epitope with which the antibody immunoreacts. The epitope specificity is defined at least in part by the amino acid residue sequence of the variable region of the heavy chain of the immunoglobulin the antibody, and in part by the light chain variable region amino acid residue sequence.

Use of the term “having the binding specificity of” indicates that equivalent monoclonal antibodies exhibit the same or similar immunoreaction (binding) characteristics and compete for binding to a preselected target epitope.

Humanized monoclonal antibodies or human monoclonal antibodies offer particular advantages over murine monoclonal antibodies, particularly insofar as they can be used therapeutically in humans.

Specifically, human antibodies are not cleared from the circulation as rapidly as “foreign” antigens, and do not activate the immune system in the same manner as foreign antigens and foreign antibodies. Methods of preparing “humanized” antibodies are generally well known in the art, and can readily be applied to the antibodies of the present invention.

Thus, the invention contemplates, in one embodiment, a monoclonal antibody of this invention that is humanized by grafting to introduce components of the human immune system without substantially interfering with the ability of the antibody to bind antigen.

The antibody of the invention can also be a fully human antibody (i.e., “humanized”) such as those generated, for example, by selection from an antibody phage display library displaying human single chain or double chain antibodies such as those described in de Haard, H. J. et al. (1999) J. Biol. Chem. 274:18218-30 and in Winter, G. et al. (1994) Annu. Rev. Immunol. 12:433-55.

In one embodiment provided herein, are antibody antagonists of ECM components such as, but mot limited to, denatured collagens. Such antibody antagonists include polyclonal antibodies, monoclonal antibodies, humanized antibodies, chimeric antibodies, genetically engineered antibodies and the like as provided above. Also provided herein are functional fragments of such antibody antagonists of ECM components including, but not limited to Fab, Fab′, F(ab′)₂, F(v), (scFv) fragments and the like. One of ordinary skill in the art would recognize that antibodies and functional fragments thereof generated against denatured ECM components could be tested using the assays provided herein for the ability to bind to denatured collagens or other respective ECM components.

Antibodies of the invention can be monoclonal or polyclonal. In one embodiment, antibodies used are monoclonal. A monoclonal antibody of this invention comprises antibody molecules that preferentially bind to denatured collagen, but bind with a substantially reduced affinity and/or avidity with the native form of the collagen. In one non-limiting example, an antibody of the invention recognizes denatured collagen type-I with an avidity at least about 2-fold, at least about 5-fold or preferably at least about 10-fold higher than that for denatured collagen type-I. In another non-limiting example, an antibody of the invention can bind to preferably to denatured collagen type-IV and binds with substantially reduced avidity to native collagen type-IV. Antibodies of the invention also can preferentially bind to each of denatured collagens types I-XXVII and bind to the native forms of each collagen with substantially reduced avidity.

Monoclonal antibodies which preferentially bind to denatured collagen include, but are not limited to, monoclonal antibodies having the immunoreaction characteristics of mAb HUI77.

Antibodies antagonists of the invention can be generated according to a number of methods known to one of skill in the art. For example, an animal can be immunized with a denatured collagen or fragment thereof. Antibodies thus generated can be selected both for their ability to bind to denatured proteolyzed collagen and for a substantially reduced affinity and/or avidity for the native form of the same collagen. Antibodies can, for example, be generated by the method of “subtractive immunization” (see, e.g., Brooks, P. C. et al. (1993) J. Cell. Biol. 122:1351-1359.)

Exemplary antagonists of denatured collagens provided herein include antagonists that preferentially bind to one or more of a sequence of an ECM component such as, for example, a denatured collagen. Exemplary sequences are those found in, for example, denatured collagen and have an amino acid sequence set forth as GPPGPP (SEQ ID NO: 81) wherein one or more proline residues is hydroxyproline. Additionally, exemplary sequences are those found in, for example, denatured collagen and have amino acid sequences set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, where P is hydroxyproline. Exemplary antagonists include, but are not limited to, antibodies or functional fragments thereof, that preferentially bind to such sequences in an EMC component (e.g., denatured collagen) and prevent binding of a cell expressing a ligand (e.g., an integrin) to the ECM component.

One non-limiting example of an antagonist provided herein is one that preferentially binds to a binding site on a denatured collagen, wherein said binding site consists essentially of an isolated polypeptide having an amino acid sequence such as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline. Alternatively, an antagonist is one that preferentially binds to a binding site on a denatured collagen, wherein said binding site consists of an isolated polypeptide having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline. In yet another embodiment, an antagonist is one that preferentially binds to a binding site on a denatured collagen, wherein said binding site consists essentially of, or consists of, a polypeptide having an amino acid sequence GPPGPP (SEQ ID NO: 81) wherein one or more proline residues is hydroxyproline. The antagonist can be an antibody or functional fragment thereof such as, for example, a polyclonal antibody, a monoclonal antibody, a chimeric antibody, a humanized antibody, a human antibody, a labeled antibody, a Fab, a F(ab)₂, a F(ab′)₂, a scFv, a genetically engineered single chain fragment, and a genetically engineered antibody. Antagonists that are antibodies or functional fragments thereof as described can inhibit angiogenesis and/or can prevent, inhibit, or treat an angiogenesis-dependent disorder, a cell proliferative disorder or a collagen-dependent disorder. Antagonists that are antibodies or functional fragments thereof as described can inhibit interactions of cell-surface integrins on tumor cells with denatured ECM components and/or can prevent, inhibit, or treat a cell-proliferative disorder or a collagen-dependent disorder.

Percentage of (%) inhibition of binding of an antibody antagonist to an ECM component of at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, or greater than negative controls is indicative of an antibody antagonist that inhibits binding of the antibody antagonist to the ECM component. Percentage (%) of inhibition of binding of the antibody antagonist to the ECM component of less than 2-fold greater than negative controls is indicative of an antibody antagonist that does not inhibit binding of the antibody antagonist to the ECM component.

B. Polypeptides, Variants Thereof and Peptidomimetics

Antagonists also can be polypeptides, peptides or peptidomimetics or variants thereof.

The terms “polypeptide” and “peptide” refer to a sequence of 3 or more amino acids connected to one another by peptide bonds between the alpha-amino group and carboxy group of contiguous amino acid residues. The term peptide as used herein refers to a linear series of two or more connected to one to the other as in a polypeptide or a cyclic peptide. Peptides include a sequence having a length from about 6 to about 12, 10 to about 20, from 18 to about 25, from 25 to about 100, from 25 to about 200, or from 50 to about 300 residues in length. Polypeptide or peptide antagonists can be, for example, a dimer, a trimer, or other multimers of the amino acid sequence. As discussed herein, antagonists that are polypeptides and peptides or variants thereof are interchangeable.

A “peptidomimetic” includes any modified form of an amino acid chain, such as a phosphorylation, capping, fatty acid modification and including unnatural backbone and/or side chain structures. A peptidomimetic comprises the structural continuum between an amino acid chain and a non-peptide small molecule. Peptidomimetics generally retain a recognizable peptide-like polymer unit structure.

Multimers of consisting of, or consisting essentially of, the polypeptides, peptides or peptidomimetics are those units that contain more than one peptide and include, for example, dimers, trimers, tetramers, etc. Where the amino acid sequence consists essentially of a reference peptide and includes no more than 20 to 30 amino acids added to the designated amino acid sequence, the multimer is increased correspondingly.

Provided herein are polypeptide antagonist is a fragment of a denatured ECM component such as, but not limited to collagen, and is capable of binding to an integrin on a cell expressing such integrin. Cells of interest are those that express integrins such as α1β1, α2β1, α3β1, α4β1, α5β1, α6β1, α7β1, α8β1, α9β1, α10β1, α11β1, αvβ1, αvβ3, αvβ5, αvβ8, αvβ6, αvβ8 and α6β4. The identification of preferred antagonist peptides having selectivity for integrins can readily be identified in a typical binding assay, such as an ELISA or ELISPOT assay.

The peptides useful in the present invention can be either linear or cyclical although cyclic peptides are preferred in some applications. Peptides or polypeptides that are in longer length, such as a length of greater than 100 amino acid residue, can be produced as a fusion protein or a fragment of a protein as described in the description of this invention. Peptides and polypeptides that are useful in this invention may not have the identical amino acid residue sequences of a denatured ECM component, and it may have that amino acid sequence as part of a longer sequence or a fusion protein, but the polypeptide or peptide is able to block binding of a cell expressing an integrin (e.g., tumor cell) to an ECM component, such as a denatured collagen.

Polypeptides and peptides of the present invention include any fragment, analog or chemical derivative of that peptide or polypeptide that has an amino acid residue sequence as shown in this application, and that the particular amino acid residue sequence, fragment or chemical derivative functions as an integrin antagonist. The polypeptides or peptides of the present invention may be a peptides or polypeptides derivative that include those residue or chemical changes including amides, conjugates with proteins, cyclic peptides, polymerized peptides and analogs of fragments of chemically modified peptides or proteins and other types of derivatives. The peptides and polypeptides useful in the present invention may include changes, substitutions, insertions and deletions where the changes in the sequence or particular chemical makeup of particular residues provide for certain advantages in the present invention. An modified integrin antagonist polypeptide peptide or peptidomimetic (e.g., variant or derivative) useful in this invention retains the ability to block binding of a cell expressing an integrin (e.g., tumor cell) to an ECM component, such as a denatured collagen. Binding and blocking of components can be determined using any of the assays as described herein or known in the art.

A peptide of the present invention may be used in the form of a pharmaceutically acceptable salt. Suitable acids which are capable of forming salts with the peptides of the present invention include inorganic acids such as trifluoroacetic acid (TFA) hydrochloric acid (HC), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl side groups (e.g., Arg, Asp, and the like) associate and neutralize each other to form an “inner salt” compound.

A peptide of the present invention can be synthesized by any of the techniques that are known to those skilled in the art, including polypeptide and recombinant DNA techniques. Synthetic chemistry techniques, such as a solid-phase Merrifield-type synthesis can be advantageous since they produce products having high purity, antigenic specificity, freedom from undesired side products, ease of production and the like. Summaries of the some techniques available can be found in, e.g., Steward et al., “Solid Phase Peptide Synthesis,” W.H. Freeman Co., San Francisco, 1969; Bodanszky, et al., “Peptide Synthesis,” John Wiley & Sons, Second Edition, 1976; J. Meienhofer, “Hormonal Proteins and Peptides,” Vol. 2, p. 46, Academic Press (New York), 1983; Merrifield, Adv. Enzymol. 1969, 32:221-96; Fields et al., Int. J. Peptide Protein Res. 1990, 35:161-214; U.S. Pat. No. 4,244,946 for solid phase peptide synthesis, and Schroder et al., “The Peptides,” Vol. 1, Academic Press (New York), 1965 (for classical solution synthesis). Such synthesis can utilize appropriate protective groups which are described in J. F. W. McOmie, “Protective Groups in Organic Chemistry,” Plenum Press, New York, 1973.

In addition, a peptide useful in the methods of this invention can be prepared without including a free ionic salt in which the charged acid or base groups present in the amino acid residue side groups (e.g., Arg, Asp, and the like) associate and neutralize each other to form an “inner salt” compound.

Solid-phase synthesis methods generally comprise the sequential addition of one or more amino acid residues or suitably protected amino acid residues to a growing peptide chain. Normally, either the amino or carboxyl group of the first amino acid residue is protected by a suitable, selectively removable protecting group. For amino acids containing a reactive side group (e.g., lysine), a different, selectively removable protecting group is utilized.

In solid phase synthesis, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next suitably protected amino acid is then added, and so forth. After all the desired amino acids have been linked in the proper sequence, any remaining terminal and side group protecting groups (and solid support) are removed sequentially or concurrently, to afford the final linear polypeptide.

Linear polypeptides may be reacted to form their corresponding cyclic peptides. A method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol, sodium hydroxide solution is added, and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography. Cyclic peptide synthesis can be achieved by alternative methods as described by Gurrath et al., Eur. J. Biochem. 1992, 210:911-921.

In addition, the antagonist can be provided in the form of a fusion protein. Fusion proteins are proteins produced by recombinant DNA methods known and described in the art, in which the subject polypeptide is expressed as a fusion with a second carrier protein such as a glutathione sulfhydryl transferase (GST) or other well-known carrier.

Thus, a polypeptide can be present in any of a variety of forms of peptide derivatives, including amides, conjugates with proteins, cyclized peptides, polymerized peptides, analogs, fragments, chemically modified peptides, peptidomimetics and like derivatives.

Suitable peptide antagonists used in the present methods are compounds that interfere with functional interactions of an integrin with a denatured ECM component. Such peptide antagonists bind to an integrin (e.g., α1β1, α2β1, α3β1, α4β1, α5β1, α6β1, α7β1, α8β1, α9β1, α10β1, α11β1, αvβ1, αvβ3, αvβ5, αvβ8, αvβ6, αvβ8 and α6β4) and interfere with binding of the integrin to an ECM component (e.g., denatured collagen). Methods for preparing and identifying certain candidate antagonists of the invention are described in, e.g., U.S. Pat. No. 6,500,924; U.S. Publication No. 2004/0063790 A1; U.S. Publication No. 2004/0258691; U.S. Publication No. 2004/0265317; U.S. Publication No. 2005/0002936, and; U.S. Publication No. 2004/0176334.

An antagonist can have the sequence characteristics of the natural denatured ECM component that interacts with the integrin. Provided herein is a peptide antagonist that is an isolated peptide consisting essentially of an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline. Also provided herein are other isolated peptides of an ECM component, e.g., denatured collagen, wherein the peptide blocks binding of an integrin to an ECM component (e.g., denatured collagen). Methods of testing binding and/or blocking have been provided elsewhere herein.

Provided herein is a peptide antagonist that is an isolated peptide consisting of an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline.

Provided herein is a peptide antagonist that is an isolated peptide consisting essentially of an amino acid sequence set forth as GPPGPP (SEQ ID NO: 81) wherein one or more proline residues is hydroxyproline. Provided herein is a peptide antagonist that is an isolated peptide consisting of an amino acid sequence set forth as GPPGPP (SEQ ID NO: 81) wherein one or more proline residues is hydroxyproline.

Also provided herein are other isolated peptides of an ECM component, e.g., denatured collagen, wherein the peptide blocks binding of an integrin to an ECM component (e.g., denatured collagen). Methods of testing binding and/or blocking have been provided elsewhere herein.

The peptide antagonist can bind to an integrin, which binding of said peptide to an integrin inhibits or prevents said integrin from binding to an extracellular matrix (ECM) component. Exemplary integrins to be blocked using peptides of the invention include, for example, β1β1, α2β1, α3β1, α4β1, α5β1, α6β1, α7β1, α8β1; α9β1, α10β1, α11β1, αvβ1, αvβ3, αvβ5, αvβ8, αvβ6, αvβ8 and α6β4.

Percentage of (%) inhibition of binding of an integrin to an ECM component by a peptide or peptidomimetic antagonist of at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, or greater than negative controls is indicative of a peptide or peptidomimetic that inhibits binding of the integrin to the ECM component. Percentage (%) of inhibition of binding of an integrin to an ECM component by a peptide or peptidomimetic antagonist of less than 2-fold greater than negative controls is indicative of a peptide or peptidomimetic that does not inhibit binding of the integrin to an ECM component.

Also provided herein are compositions of the peptide antagonists provided herein and a pharmaceutically acceptable carrier/excipient.

In a separate aspect, the present invention provides for a pharmaceutically acceptable salt of any of the peptides provided herein.

Peptide antagonists can be modified to (i) make the peptide more immunogenic via fusions to other moieties as described elsewhere herein, (ii) providing the peptide with a therapeutic moiety, (iii) providing the peptide with a diagnostic or imaging moiety. Providing the peptide with another moiety can be accomplished by administering both components at the same time in separate compositions, combining both components in the same composition, or linking the components. Methods of covalently and non-covalently linking components are well-known in the art.

In one embodiment, the composition is lyophilized. When the compositions are considered for pharmaceutical compositions, medicaments, or use in any of the methods provided herein, it is contemplated that the composition will be substantially free of pyrogens such that the composition will not cause an inflammatory reaction.

Peptide and polypeptide antagonists can be generated by a number of techniques known to one of skill in the art. For example, a two hybrid system (e.g., Fields, S. (1989) Nature 340:245-6) can use a fragment of a collagen as “bait” for selecting protein antagonists from a library that bind to the collagen peptide. In another example, the two hybrid system can use a fragment of an integrin as “bait” for selecting protein antagonists from a library that bind to the integrin. The library of potential antagonists can be derived from a cDNA library, for example. In another embodiment, the potential antagonists can be variants of known collagen binding proteins. Such proteins can be randomly mutagenized or subjected to gene shuffling, or other available techniques for generating sequence diversity.

Peptide and polypeptide antagonists of the invention also can be generated by techniques of molecular evolution. Libraries of proteins can be generated by mutagenesis, gene shuffling or other available techniques for generating molecular diversity. In one embodiment, protein pools representing numerous variants can be selected for their ability to bind to denatured collagen, for instance by passing such protein pools over a solid matrix to which a denatured collagen has been attached. Elution with gradients of salt, for example, can provide purification of variants with affinity for the denatured collagen. A negative selection step also can be included whereby such pools are passed over a solid matrix to which native collagens have been attached. The filtrate will contain those variants within the pool that have a reduced affinity for the native form of the collagen. In another embodiment, protein pools representing numerous variants can also be selected for their ability to bind to an integrin, for instance by passing such protein pools over a solid matrix to which an integrin has been attached. Elution with gradients of salt, for example, can provide purification of variants with affinity for the integrin.

Peptide and polypeptide antagonists of the invention also can be generated by phage display. A randomized peptide or protein can be expressed on the surface of a phagemid particle as a fusion with a phage coat protein. Techniques of monovalent phage display are widely available (see, e.g., Lowman H. B. et al. (1991) Biochemistry 30:10832-8.)

In one example, phage expressing randomized peptide or protein libraries can be panned with a solid matrix to which a native collagen molecule has been attached. Remaining phage do not bind native collagens, or bind native collagens with substantially reduced affinity. The phage are then panned against a solid matrix to which a denatured collagen has been attached. Bound phage are isolated and separated from the solid matrix by either a change in solution conditions or, for a suitably designed construct, by proteolytic cleavage of a linker region connecting the phage coat protein with the randomized peptide or protein library. The isolated phage can be sequenced to determine the identity of the selected antagonist.

In one example, phage expressing randomized peptide or protein libraries can be panned with a solid matrix to which an integrin has been attached. Bound phage are isolated and separated from the solid matrix by either a change in solution conditions or, for a suitably designed construct, by proteolytic cleavage of a linker region connecting the phage coat protein with the randomized peptide or protein library. The isolated phage can be sequenced to determine the identity of the selected antagonist.

In another embodiment, a polypeptide includes any analog, fragment or chemical derivative of a polypeptide whose amino acid residue sequence is shown herein so long as the polypeptide is an antagonist of denatured collagen, but not of native collagen. Alternatively, a polypeptide includes any analog, fragment or chemical derivative of a polypeptide whose amino acid residue sequence is shown herein so long as the polypeptide is an antagonist of an integrin.

Therefore, a present polypeptide can be subject to various changes, substitutions, insertions, and deletions where such changes provide for certain advantages in its use. In this regard, a denatured collagen antagonist polypeptide of this invention corresponds to, rather than is identical to, the sequence of a recited peptide where one or more changes are made and it retains the ability to function as a denatured collagen antagonist in one or more of the assays as defined herein. Alternatively, an integrin antagonist polypeptide of this invention corresponds to, rather than is identical to, the sequence of a recited peptide where one or more changes are made and it retains the ability to function as an integrin antagonist in one or more of the assays as defined herein.

Thus, a polypeptide can be in any of a variety of forms of a peptide derivative that include amides, conjugates with proteins, cyclized peptides, polymerized peptides, analogs, fragments, chemically modified peptides, and like derivatives.

Peptides can also be synthesized and expressed as fusion proteins with one or more additional domains linked thereto for producing a more immunogenic peptide, to more readily isolate a recombinantly synthesized peptide, or to identify and isolate antibodies or antibody-expressing B cells. Domains facilitating detection and purification include, for example, metal chelating peptides such as polyhistidine tracts and histidine-tryptophan modules that allow purification on immobilized metals; protein A domains that allow purification on immobilized immunoglobulin; and the domain utilized in the FLAGS® extension/affinity purification system (Immunex Corp, Seattle, Wash.) The inclusion of a cleavable linker sequence such as Factor Xa or enterokinase (Invitrogen, San Diego Calif.) between a purification domain and the peptide can be used to facilitate peptide purification. For example, an expression vector can include a peptide-encoding nucleic acid sequence linked to six histidine residues followed by a thioredoxin and an enterokinase cleavage site (see e.g., Williams (1995) Biochemistry 34:1787 1797; Dobeli (1998) Protein Expr. Purif. 12:404 14). The histidine residues facilitate detection and purification of the fusion protein while the enterokinase cleavage site provides a means for purifying the peptide from the remainder of the fusion protein. Technology pertaining to vectors encoding fusion proteins and application of fusion proteins is known in the art (see e.g., Kroll (1993) DNA Cell. Biol. 12: 441-453).

The present invention provides for ECM component peptidomimetics. The peptidomimetics will act to bind to a ligand (e.g., an integrin) of an ECM component (e.g., denatured collagen) to form a complex which prevents binding of the ligand to its natural ECM component.

In certain embodiments, a subject therapeutic comprises a peptidomimetic of a peptide. Peptidomimetics are compounds based on, or derived from, peptides and proteins. The peptidomimetics of the present invention typically can be obtained by structural modification of a known peptide sequence using one or more unnatural amino acids, conformational restraints, isosteric replacements, and the like. The subject peptidomimetics constitute the continuum of structural space between peptides and non-peptide synthetic structures; peptidomimetics may be useful, therefore, in delineating pharmacophores and in helping to translate peptides into non-peptide compounds with the activity of the parent peptides.

Peptidomimetics can have such attributes as being non-hydrolyzable (e.g., increased stability against proteases or other physiological conditions which degrade the corresponding peptide) or increased specificity and/or potency for binding to a ligand of an ECM component to form a complex which prevents binding of the ligand to its natural ECM component. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988, p 123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al. (1985) Tetrahedron Letters 26:647; and Sato et al. (1986) J. Chem. Soc. Perkin. Trans. 1:1231), b-aminoalcohols (Gordon et al. (1985) Biochem. Biophys. Res. Commun. 126:419; and Dann et al. (1986) Biochem. Biophys. Res. Commun. 134:71), diaminoketones (Natarajan et al. (1984) Biochem. Biophys. Res. Commun. 124:141), methyleneamino-modified (Roark et al. in Peptides: Chemistry and Biology, G. R. Marshall ed., ESCOM Publisher: Leiden, Netherlands, 1988, p 134), vinylogous polypeptides (Hagihara et al. (1992) J. Am. Chem. Soc. 114: 6568-70), oligoanthranilamides (Hamuro et al. (1996) J. Am. Chem. Soc. 118: 7529-41), vinylogous sulfonaminopeptides (Genarri et al. (1996) Chem. Eur. J. 2: 644-55), aedemers (Lokey et al. (1995) Nature 375: 303-5), and sugar-based peptidomimetics (Horvat et al. (1998) J. Chem. Soc. Perkins Trans. 1: 1789-95). Also, see generally, Session III: Analytic and synthetic methods, in Peptides: Chemistry and Biology, G. R. Marshall ed., ESCOM Publisher: Leiden, Netherlands, 1988). In addition, U.S. Pat. No. 5,422,426 describes high throughput and combinatorial methods for producing peptides and peptidomimetics.

In further embodiments, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides and (v) sulfonamides.

Additionally, peptidomimetics based on more substantial modifications of the backbone of the peptide can be used. Peptidomimetics which fall in this category include (i) retro-inverso analogs, and (ii) N-alkyl glycine analogs (so-called peptoids, see e.g. Simon et al. (1992) Proc. Natl. Acad. Sci. USA 89: 9367-71) and others mentioned above.

Such retro-inverso analogs can be made according to the methods known in the art, such as that described by the Sisto et al. U.S. Pat. No. 4,522,752. For example, a retro-inverso analog can be generated as follows. The first step is to form a geminal diamine analog of an amino acid. The geminal diamine corresponding to the N-terminal amino acid, for example an N-terminal proline is synthesized by treating an N-protected proline analog with ammonia under HOBT-DCC coupling conditions to yield an unsubstituted amide, and then effecting a Hofmann-type rearrangement with I,I-bis-(trifluoroacetoxy)iodobenzene (TIB), as described in Radhakrishna et al. (1979) J. Org. Chem. 44:1746, effectively removing the carbonyl moiety from the unsubstituted amide. The product amine is then coupled to a side-chain protected (e.g., as the benzyl ester) second amino acid, such as N-Fmoc D-Thr residue under standard conditions to yield the pseudodipeptide. The Fmoc (fluorenylmethoxycarbonyl) group is removed with piperidine in dimethylformamide, and the resulting amine is trimethylsilylated with bistrimethylsilylacetamide (BSA) before condensation with suitably alkylated, side-chain protected derivative of Meldrum's acid as described in U.S. Pat. No. 5,061,811 (Pinori et al.) to yield a retro-inverso tripeptide. Meldrum's acid is a cyclic malonate analog where the R substitution on the fifth carbon (the carbon between the carbonyl groups) determines the amino acid moiety added. For instance, if the R group in Meldrum's acid is a methyl group, then an alanine moiety is added and the retro-inverso tripeptide has the formula PTA. The remaining ester group from the ring opening reaction with Meldrum's acid is further coupled with an amino acid analog under standard conditions to give the protected tetrapeptide analog. The protecting groups are removed to release the product and the steps repeated to elongate the tetrapeptide to the full length peptidomimetic. It will be generally understood that a mixed peptide, e.g. including some normal peptide linkages, will be generated by this process. As a general guide, sites which are most susceptible to proteolysis are typically altered, with less susceptible amide linkages being optional for mimetic switching. The final product, or intermediates thereof, can be purified by HPLC. Another embodiment involves building the retro-inverso tetrapeptide from the C-terminus using similar protective chemistry techniques.

Retro-enantio analogs such as this can be synthesized commercially available D-amino acids (or analogs thereof) and standard solid- or solution-phase peptide-synthesis techniques. For example, in a preferred solid-phase synthesis method, a suitably amino-protected (t-butyloxycarbonyl, Boc) residue (e.g. D-proline) is covalently bound to a solid support such as chloromethyl resin. The resin is washed with dichloromethane (DCM), and the BOC protecting group removed by treatment with TFA in DCM. The resin is washed and neutralized, and the next Boc-protected D-amino acid (e.g. D-Thr) is introduced by coupling with diisopropylcarbodiimide. The resin is again washed, and the cycle repeated for each of the remaining amino acids in turn (D-Ala, D-Pro, etc). When synthesis of the protected retroenantio peptide is complete, the protecting groups are removed and the peptide cleaved from the solid support by treatment with hydrofluoric acid/anisole/dimethyl sulfide/thioanisole. The final product is purified by HPLC to yield the pure retro-enantio analog.

The trans olefin analog of a peptide can be synthesized according to the method of Y. K. Shue et al. (1987) Tetrahedron Letters 28:3225. Other pseudo-dipeptides can be made by the method set forth above merely by substitution of the appropriate starting Boc amino acid and Wittig reagent. Variations in the procedure may be necessary according to the nature of the reagents used, but any such variations will be purely routine and will be apparent to one of skill in the art.

The synthesis of phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the subject peptidomimetics. To illustrate, the peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

In certain embodiments, the subject peptide or peptidomimetic is capped at either the N-terminus (or other end structure at the normal N-terminus such as a carbonyl), the C-terminus (or other end structure at the normal C-terminus, such as an amine), or both. For example, a therapeutic may comprise a polypeptide comprising one or more sequences where the polypeptide has either an acetyl cap at the N-terminus, an amide cap at the C-terminus, or both. Methods for synthesizing capped peptides are disclosed, for example, in U.S. Pat. No. 5,994,309.

The subject peptidomimetics can be optimized by, e.g., combinatorial synthesis techniques combined with such high throughput screening as described herein.

Moreover, other examples of peptidomimetics include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A peptidomimetic can be obtained by, for example, screening libraries of natural and synthetic compounds for compounds capable of binds to a ligand of an ECM component to form a complex which prevents binding of the ligand to an ECM component. A peptidomimetic can also be obtained, for example, from libraries of natural and synthetic compounds, in particular, chemical or combinatorial libraries (i.e., libraries of compounds that differ in sequence or size but that have the same building blocks).

In certain aspects, subject therapeutic molecules, such as peptidomimetics and small molecules, may be obtained by rational design. In an exemplary rational design procedure, the three-dimensional structure of a target peptide of the present invention can be analyzed by, for example, nuclear magnetic resonance (NMR) or x-ray crystallography. The three-dimensional structure can then be used to predict structures of potential therapeutics by, for example, computer modeling. The predicted therapeutic structures can then be produced by, for example, chemical synthesis, recombinant DNA technology, or by isolating a molecule from a natural source (e.g., plants, animals, bacteria and fungi). Accordingly, a therapeutic may be modeled on an unconstrained polypeptide chain of one of the above-mentioned sequences. Where the target peptide is unconstrained, the desired structure may be modeled without resort to NMR or X-ray crystallography, and any of a number of programs for modeling short peptide chains may be employed.

In certain embodiments, peptides, peptidomimetics and small molecules are able to bind to a ligand of an ECM component to form a complex which prevents binding of the ligand to its natural ECM component. Optionally, peptides, peptidomimetics and small molecules have a K_{D} that is no more than ten times greater than the K_{D} of the peptide on which they are based, and optionally have a roughly equivalent K_{D} or a K_{D} ten times lower or less.

C. Other Antagonists

Antagonists of the invention also can be small organic molecules, such as those natural products, or those compounds synthesized by conventional organic synthesis or combinatorial organic synthesis. Compounds can be tested for their ability to bind to a denatured collagen, for example, by using the column binding technique described above. Compounds also are selected for reduced affinity for the native form of the collagen by a similar column binding technique. Compounds can be tested for their ability to bind to an integrin by using the column binding technique described above.

Antagonists of the invention also can be non-peptidic compounds. Suitable non-peptidic compounds include, for example, oligonucleotides. Oligonucleotides as used herein refer to any heteropolymeric material containing purine, pyrimidine and other aromatic bases. DNA and RNA oligonucleotides are suitable for use with the invention, as are oligonucleotides with sugar (e.g., 2′ alkylated riboses) and backbone modifications (e.g., phosphorothioate oligonucleotides). Oligonucleotides may present commonly found purine and pyrimidine bases such as adenine, thymine, guanine, cytidine and uridine, as well as bases modified within the heterocyclic ring portion (e.g., 7-deazaguanine) or in exocyclic positions. Oligonucleotide also encompasses heteropolymers with distinct structures that also present aromatic bases, including polyamide nucleic acids and the like.

An oligonucleotide antagonist of the invention can be generated by a number of methods known to one of skill in the art. In one embodiment, a pool of oligonucleotides is generated containing a large number of sequences. Pools can be generated, for example, by solid phase synthesis using mixtures of monomers at an elongation step. The pool of oligonucleotides is sorted by passing a solution containing the pool over a solid matrix to which a denatured collagen or fragment thereof has been affixed. Sequences within the pool that bind to the denatured collagen are retained on the solid matrix. These sequences are eluted with a solution of different salt concentration or pH. Sequences selected are subjected to a second selection step. The selected pool is passed over a second solid matrix to which native collagen has been affixed. The column retains those sequences that bind to the native collagen, thus enriching the pool for sequences specific for the denatured collagen. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli. In another embodiment, the pool of oligonucleotides is sorted by passing a solution containing the pool over a solid matrix to which an integrin or fragment thereof has been affixed. Sequences within the pool that bind to the integrin are retained on the solid matrix. These sequences are eluted with a solution of different salt concentration or pH. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli.

Provided herein are antagonists of other ECM components including, but not limited to, those provided in Tables 1 through 8.

D. Linkers

It may be necessary in some instances to introduce an unstructured polypeptide linker region between a label of the present invention and portions of the antagonists. The linker can facilitate enhanced flexibility, and/or reduce steric hindrance between any two fragments. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the 1CI and LexA proteins.

Within the linker, the amino acid sequence may be varied based on the preferred characteristics of the linker as determined empirically or as revealed by modeling. Considerations in choosing a linker include flexibility of the linker, charge of the linker, and presence of some amino acids of the linker in the naturally-occurring subunits. The linker can also be designed such that residues in the linker contact DNA, thereby influencing binding affinity or specificity, or to interact with other proteins. In some cases, particularly when it is necessary to span a longer distance between subunits or when the domains must be held in a particular configuration, the linker may optionally contain an additional folded domain.

In some embodiments it is preferable that the design of a linker involve an arrangement of domains which requires the linker to span a relatively short distance, preferably less than about 10 Angstroms (Å). However, in certain embodiments, linkers span a distance of up to about 50 Å.

E. Labels

Antagonists provided herein such that they are conjugated or linked to therapeutic and/or imaging/detectable moieties. Methods for conjugating or linking proteins, peptides, peptidomimetics, antibodies and fragments thereof are well known in the art. Associations between antagonists and labels include any means known in the art including, but not limited to, covalent and non-covalent interactions.

In one non-limiting embodiment, an antagonist can be associated with a toxin, a radionuclide, an iron-related compound, a dye, an imaging reagent, a fluorescent label or a chemotherapeutic agent that would be toxic when delivered to a cancer cell.

Alternatively, the antagonists can be associated with detectable label, such as a radionuclide, iron-related compound, a dye, an imaging agent or a fluorescent agent for immunodetection of target antigens.

1. Radiolabels

Non-limiting examples of radiolabels include, for example, ³²P, ³³P, ⁴³K, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹ Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi.

2. Toxins

Non-limiting examples of toxins include, for example, ricin A chain (ricin toxin), Pseudomonas exotoxin (PE), diphtheria toxin (DT), Clostridium perfringens phospholipase C (PLC), bovine pancreatic ribonuclease (BPR), antiviral protein (PAP), abrin, abrin A chain (abrin toxin), cobra venom factor (CVF), gelonin (GEL), saporin (SAP) and viscumin.

Non-limiting examples of iron-related compounds include, for example, magnetic iron-oxide particles, ferric or ferrous particles, Fe²⁰³ and Fe³⁰⁴. Iron-related compounds and methods of labeling polypeptides, proteins and peptides can be found, for example, in U.S. Pat. Nos. 4,101,435 and 4,452,773, and U.S. published applications 20020064502 and 20020136693, all of which are hereby incorporated by reference in their entirety.

In certain embodiments, the subject antagonists can be covalently or non-covalently coupled to a cytotoxin or other cell proliferation inhibiting compound, in order to localize delivery of that agent to a tumor cell. For instance, the agent can be selected from the group consisting agents, enzyme inhibitors, proliferation inhibitors, lytic agents, DNA or RNA synthesis inhibitors, membrane permeability modifiers, DNA metabolites, dichloroethylsulfide derivatives, protein production inhibitors, ribosome inhibitors, inducers of apoptosis, and neurotoxins.

3. Imaging Agents

In certain embodiments, the subject antagonists can be coupled with an agent useful in imaging tumors. Such agents include: metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores. In many embodiments, such secondary functionality/moiety will be relatively large, e.g., at least 25 amu in size, and in many instances can be at least 50,100 or 250 amu in size.

In certain preferred embodiments, the secondary functionality is a chelate moiety for chelating a metal, e.g., a chelator for a radiometal or paramagnetic ion. In preferred embodiments, it is a chelator for a radionuclide useful for radiotherapy or imaging procedures.

Radionuclides useful within the present invention include gamma-emitters, positron-emitters, Auger electron-emitters, X-ray emitters and fluorescence-emitters, with beta- or alpha-emitters preferred for therapeutic use. Examples of radionuclides useful as toxins in radiation therapy include: ³²P, ³³P, ⁴³K, ⁵²Fe, ⁵⁷Co, ⁶⁴Cu, ⁶⁴Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu. Conditions under which a chelator will coordinate a metal are described, for example, by Gasnow et al. U.S. Pat. Nos. 4,831,175, 4,454,106 and 4,472,509, each of which is incorporated herein by reference. Within the present invention, “radionuclide” and “radiolabel” are interchangeable.

⁹⁹Tc is a particularly attractive radioisotope for diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain preferred embodiments, the modified antagonists include a chelating agent for technium.

In still other embodiments, the secondary functionality can be a radiosensitizing agent, e.g., a moiety that increases the sensitivity of cells to radiation. Examples of radiosensitizing agents include nitroimidazoles, metronidazole and misonidazole (see: DeVita, V. T. in Harrison's Principles of Internal Medicine, p. 68, McGraw-Hill Book Co., NY, 1983, which is incorporated herein by reference). The modified antagonists that comprise a radiosensitizing agent as the active moiety are administered and localize at the target cell. Upon exposure of the individual to radiation, the radiosensitizing agent is “excited” and causes the death of the cell.

There are a wide range of moieties which can serve as chelators and which can be derivatized to the antagonists of the present invention. For instance, the chelator can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to subject antagonists. Such groups include, e.g., benzylisothiocyanate, by which the DOTA, DTPA or EDTA can be coupled to, e.g., an amine group.

In one embodiment, the chelate moiety is an “NxSy” chelate moiety. As defined herein, the “NxSy chelates” include bifunctional chelators that are capable of coordinately binding a metal or radiometal and, preferably, have N2S2 or N3S cores. Exemplary NxSy chelates are described, e.g., in Fritzberg et al. (1998) PNAS 85: 4024-29; and Weber et al. (1990) Chem. 1: 431-37; and in the references cited therein.

The Jacobsen et al. PCT application WO 98/12156 provides methods and compositions, i.e. synthetic libraries of binding moieties, for identifying compounds which bind to a metal atom. The approach described in that publication can be used to identify binding moieties which can subsequently be added to the antagonists to derive the modified antagonists.

A problem frequently encountered with the use of conjugated proteins in and radiodiagnostic applications is a potentially dangerous accumulation of the radiolabeled moiety fragments in the kidney. When the conjugate is formed using an acid- or base-labile linker, cleavage of the radioactive chelate from the protein can advantageously occur. If the chelate is of relatively low molecular weight, as most of the subject modified antibodies, antigen binding fragments and peptides are expected to be, it is not retained in the kidney and is excreted in the urine, thereby reducing the exposure of the kidney to radioactivity. However, in certain instances, it may be advantageous to utilize acid- or base-labile in the subject ligands for the same reasons they have been used in labeled proteins.

Accordingly, certain of the subject labeled/modified antagonists can be synthesized, by standard methods in the art, to provide reactive functional groups which can form acid-labile linkages with, e.g., a carbonyl group of the ligand. Examples of suitable acid-labile linkages include hydrazone and thiosemicarbazone functions. These are formed by reacting the oxidized carbohydrate with chelates bearing hydrazide, thiosemicarbazide, and functions, respectively.

Alternatively, base-cleavable which have been used for the enhanced clearance of the radiolabel from the kidneys, can be used. See, for example, Weber et al. 1990 Bioconjg. Chem. 1:431. The coupling of a bifunctional chelate to an antagonist via a hydrazide linkage can incorporate base-sensitive ester moieties in a linker spacer arm. Such an ester-containing linker unit is exemplified by ethylene glycolbis (succinimidyl succinate), (EGS, available from Pierce Chemical Co., Rockford, Ill.), which has two terminal N-hydroxysuccinimide (NHS) ester derivatives of two 1,4-dibutyric acid units, each of which are linked to a single ethylene glycol moiety by two alkyl esters. One NHS ester may be replaced with a suitable amine-containing BFC (for example 2-aminobenzyl DTPA), while the other NHS ester is reacted with a limiting amount of hydrazine. The resulting hyrazide is used for coupling to the antagonists, forming an ligand-BFC linkage containing two alkyl ester functions. Such a conjugate is stable at physiological pH, but readily cleaved at basic pH.

Antagonists labeled by are subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, the antagonists are coupled to a Boron addend, such as a carborane. For example, carboranes can be prepared with carboxyl functions on pendant side chains, as is well known in the art. Attachment of such carboranes to amine peptides can be achieved by activation of the carboxyl groups of the carboranes and condensation with the amine group to produce the conjugate. Such modified antagonists can be used for neutron capture therapy.

4. Dyes

The present invention also contemplates the modification of the subject antagonists with dyes, for example, useful in therapy, and used in conjunction with appropriate non-ionizing radiation. The use of light and porphyrins in methods of the present invention is also contemplated and their use in cancer therapy has been reviewed by van den Bergh, Chemistry in Britain, 22: 430-437 (1986), which is incorporated by reference herein in its entirety.

5. Fluorescent Labels

One embodiment of the present invention includes antagonists labeled with a fluorescent label. Common fluorescent labels include, for example, FITC, PE, Texas Red, cytochrome c, etc. Techniques for labeling polypeptides and fragments thereof, such as those provided herein, are well-known in the art.

6. Anti-Cancer Agents

Chemotherapeutics useful as active moieties which when conjugated to antagonists thereof of the present invention are specifically delivered to cells are typically, small chemical entities produced by chemical synthesis. Chemotherapeutics include cytotoxic and cytostatic drugs. Chemotherapeutics may include those which have other effects on cells such as reversal of the transformed state to a differentiated state or those which inhibit cell replication. Examples of known cytotoxic agents useful in the present invention are listed, for example, in Goodman et al., “The Pharmacological Basis of Therapeutics,” Sixth Edition, A. B. Gilman et al., eds./Macmillan Publishing Co. New York, 1980. These include taxanes, such as paclitaxel and docetaxel; nitrogen such as mechlorethamine, melphalan, uracil mustard and chlorambucil; ethylenimine derivatives, such as thiotepa; alkyl sulfonates, such as busulfan; nitrosoureas, such as lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, and mitomycin; enzymes, such as platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, acetate and megestrol acetate), estrogens (diethylstilbestrol and ethinyl estradiol), and androgens (testosterone propionate and fluoxymesterone).

Drugs that interfere with protein synthesis can also be used; such drugs are known to those skilled in the art and include puromycin, cycloheximide, and ribonuclease.

Most of the chemotherapeutic agents currently in use in treating cancer possess functional groups that are amenable to chemical cross-linking directly with an amine or carboxyl group of an agent of the present invention. For example, free amino groups are available on methotrexate, doxorubicin, daunorubicin, cytosinarabinoside, bleomycin, fludarabine, and cladribine while free carboxylic acid groups are available on methotrexate, melphalan, and chlorambucil.

These functional groups, that is free amino and carboxylic acids, are targets for a variety of homobifunctional and heterobifunctional chemical cross-linking agents which can crosslink these drugs directly to a free amino group of an antagonist.

Chemotherapeutic agents contemplated by the present invention also include other chemotherapeutic drugs that are commercially available. Merely to illustrate, the chemotherapeutic can be an inhibitor of chromatin function, a inhibitor, a inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), a DNA repair inhibitor.

Chemotherapeutic agents may be categorized by their mechanism of action into, for example, the following groups: anti-metabolites/anti-cancer agents, such as pyrimidine analogs floxuridine, capecitabine, and cytarabine) and purine analogs, folate antagonists and related inhibitors antiproliferative/antimitotic agents including natural products such as vinca alkaloid (vinblastine, vincristine, and microtubule such as taxane (paclitaxel, docetaxel), vinblastin, nocodazole, epothilones and navelbine, epidipodophyllotoxins (etoposide, teniposide), DNA damaging agents (actinomycin, amsacrine, busulfan, carboplatin, chlorambucil, cisplatin, cyclophosphamide, cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, triethylenethiophosphoramide and etoposide. (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prenisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin. Preferred dosages of the chemotherapeutic agents are consistent with currently prescribed dosages.

Additionally, other labels, such as biotin followed by streptavidin-alkaline phosphatase (AP), horseradish peroxidase are contemplated by the present invention.

As used herein, the terms “nucleic acid damaging treatment” and “nucleic acid damaging agent” means any treatment regimen that directly or indirectly damages nucleic acid (e.g., DNA, cDNA, genomic DNA, mRNA, tRNA or rRNA). Examples of such agents include alkylating agents, nitrosoureas, anti-metabolites, plant alkaloids, plant extracts and radioisotopes. Examples of agents also include nucleic acid damaging drugs, for example, 5-fluorouracil (5-FU), capecitabine, S-1 (Tegafur, 5-chloro-2,4-dihydroxypyridine and oxonic acid), 5-ethynyluracil, arabinosyl cytosine (ara-C), 5-azacytidine (5-AC), 2′,2′-difluoro-2′-deoxycytidine (dFdC), purine antimetabolites (mercaptopurine, azathiopurine, thioguanine), gemcitabine hydrochloride (Gemzar), pentostatin, allopurinol, 2-fluoro-arabinosyl-adenine (2F-ara-A), hydroxyurea, sulfur mustard (bischloroetyhylsulfide), mechlorethamine, melphalan, chlorambucil, cyclophosphamide, ifosfamide, thiotepa, AZQ, mitomycin C, dianhydrogalactitol, dibromoducitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubibcin (Cerubicine), idarubicin (Idamycin) and epirubicin (Ellence), anthracyclin analogues such as mitoxantrone, actinomycin D, non intercalating topoisomerase inhibitors such as epipodophyllotoxins (etoposide=VP16, teniposide=VM-26), podophylotoxin, bleomycin (Bleo), pepleomycin, compounds that form adducts with nucleic acid including platinum derivatives (e.g., cisplatin (CDDP), trans analogue of cisplatin, carboplatin, iproplatin, tetraplatin and oxaliplatin), camptothecin, topotecan, irinotecan (CPT-11), and SN-38. Specific examples of nucleic acid damaging treatments include radiation (e.g., focused microwaves, ultraviolet (UV), infrared (IR), or alpha-, beta- or gamma-radiation) and environmental shock (e.g., hyperthermia).

As used herein, the terms “anti-proliferative treatment” and “anti-proliferative agent” means any treatment regimen that directly or indirectly inhibits proliferation of a cell, virus, bacteria or other unicellular or multicellular organism regardless of whether or not the treatment or agent damages nucleic acid. Particular examples of anti-proliferative agents are anti-tumor and anti-viral drugs, which inhibit cell proliferation or virus proliferation or replication. Examples include, inter alia, cyclophosphamide, azathioprine, cyclosporin A, prednisolone, melphalan, chlorambucil, mechlorethamine, busulphan, methotrexate, 6-mercaptopurine, thioguanine, cytosine arabinoside, taxol, vinblastine, vincristine, doxorubicin, actinomycin D, mithramycin, carmustine, lomustine, semustine, streptozotocin, hydroxyurea, cisplatin, mitotane, procarbazine, dacarbazine and dibromomannitol. Anti proliferative agents that cause nucleic acid replication errors or inhibit nucleic acid replication are those such as nucleoside and nucleotide analogues (e.g., AZT or 5-AZC).

Methodology for labeling polypeptides and fragments thereof including, but not limited to, those provided herein are well known in the art. When the antagonists of the present invention are labeled with a radiolabel or toxin, the antagonists can be prepared as pharmaceutical compositions which are useful for therapeutic treatment of patients exhibiting tumors or angiogenesis or the like where the pharmaceutical compositions are administered to the patient in an effective amount. When the antagonists of the present invention are labeled with a label that can be visualized, the antagonists can be prepared as pharmaceutical compositions which are useful for diagnostic of patients where the pharmaceutical compositions are administered to the patient in an effective amount for in vivo imaging or where the pharmaceutical compositions are tested in an in vitro assay.

II. Identification of Antagonists of Integrins

Antagonists are evaluated for their ability to bind to an integrin, and furthermore can be evaluated for their ability to inhibit binding of an integrin to a denatured ECM component (e.g., denatured collagen). In one embodiment, the integrin is expressed on a cell such as a tumor or metastatic cell. As used herein, references to an “integrin” refer to a recombinantly expressed polypeptide or one that is expressed on a cell and integrins produced by either method are used interchangeably. Measurement of binding of antagonists to an integrin and their ability to inhibit binding of an integrin to denatured ECM molecules can be accomplished, e.g., using an enzyme-linked-immunosorbent assay (ELISA), an ELISOPT assay or any other conventional assay known in the art. The ELISA and ELISPOT are commonly used and well-known to those of skill in the art.

The ELISA also can be used to identify compounds which exhibit increased specificity for an integrin in comparison to other molecules. The specificity assay is conducted by running parallel ELISAs in which a potential antagonist is screened concurrently in separate assay chambers for the ability to bind an integrin. Another technique for measuring apparent binding affinity familiar to those of skill in the art is a surface plasmon resonance technique (analyzed on a BIACORE 2000 system) (Liljeblad, et al., Glyco. J. 2000, 17:323-329). Standard measurements and traditional binding assays are described by Heeley, R. P., Endocr. Res. 2002, 28:217-229.

Antagonists of integrins can also be identified by their ability to compete for binding with an antagonist useful in the present invention. Such antagonists likely have the same specificity as, and recognize the same epitope, as the antibody itself.

Antagonists can also be identified by their ability to bind to a solid matrix containing an integrin. Such putative antagonists are collected after altering solution conditions, such as salt concentration, pH, temperature, etc. The putative antagonists are further identified by their ability to pass through, under appropriate solution conditions, a solid matrix to which the integrin has been affixed.

Antagonists useful in the invention also can be assayed for their ability to influence tumor development processes, e.g., angiogenesis, tumor metastasis, cell adhesion, cell migration, cell proliferation, and tumor growth in a tissue. Any suitable assay known to one of skill in the art can be used to monitor such effects. Several such techniques are described herein.

Exemplary peptide antagonists (and variants or peptidomimetics) are fragments of denatured ECM components (e.g., denatured collagen) that are capable of binding to an integrin. Such peptide antagonists include, but are not limited to, peptides consisting essentially of, or consisting of, a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline. Other peptide antagonists include, but are not limited to, peptides consisting essentially of, or consisting of, a sequence of amino acids set forth as GPPGPP (SEQ ID NO: 81) wherein one or more proline residues is hydroxyproline.

III. Identification of Antagonists of Denatured ECM Components

Provided herein are methods of identifying antagonists of denatured extracellular matrix (ECM) components. Antagonists are evaluated for their ability to bind denatured ECM components (e.g., denatured) collagens, and furthermore can be evaluated for their ability to inhibit binding of an integrin to the denatured ECM components. Measurement of binding of antagonists to denatured ECM components can be accomplished, e.g., using an enzyme-linked-immunosorbent assay (ELISA), described in the publications listed herein. The ELISA is commonly used and well-known to those of skill in the art.

The ELISA also can be used to identify compounds which exhibit increased specificity for one or more denatured collagens in comparison to other molecules or to a native collagen. The specificity assay is conducted by running parallel ELISAs in which a potential antagonist is screened concurrently in separate assay chambers for the ability to bind one or more denatured collagens. Another technique for measuring apparent binding affinity familiar to those of skill in the art is a surface plasmon resonance technique (analyzed on a BIACORE 2000 system) (Liljeblad, et al., Glyco. J. 2000, 17:323-329). Standard measurements and traditional binding assays are described by Heeley, R. P., Endocr. Res. 2002, 28:217-229.

Antagonists of denatured collagen can also be identified by their ability to compete for binding with an antagonist useful in the present invention. For example, putative antagonists can be screened by monitoring their effect on the affinity of a known antagonist, such as antibody HUI77, described, e.g., in pending U.S. Ser. No. 10/011,250, the subject matter of which is incorporated herein in its entirety by reference.

Antagonists can also be identified by their ability to bind to a solid matrix containing a denatured ECM component. Such putative antagonists are collected after altering solution conditions, such as salt concentration, pH, temperature, etc. The putative antagonists are further identified by their ability to pass through, under appropriate solution conditions, a solid matrix to which a denatured ECM component has been affixed.

Antagonists useful in the invention also can be assayed for their ability to influence tumor development processes, e.g., angiogenesis, tumor metastasis, cell adhesion, cell migration, cell proliferation, and tumor growth in a tissue. Any suitable assay known to one of skill in the art can be used to monitor such effects. Several such techniques are described herein.

Exemplary antibody (or functional fragments thereof) antagonists provided herein, are those that bind to a peptide of a denatured ECM component. Such antibody antagonists include, but are not limited to, antibodies and functional fragments thereof that preferentially bind to peptides consisting essentially of, or consisting of, a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant or peptidomimetic thereof, wherein P is hydroxyproline. Such antibody antagonists also include, but are not limited to, antibodies and functional fragments thereof that preferentially bind to peptides consisting essentially of, or consisting of, a sequence of amino acids set forth as GPPGPP (SEQ ID NO: 81) wherein one or more proline residues is hydroxyproline.

IV. Compositions and Medicaments

Each of the antagonists of the present invention can be used as a composition when combined with a pharmaceutically acceptable carrier or excipient. Such pharmaceutical compositions are useful for administration to a subject in vivo or ex vivo, and for diagnosing and/or treating a subject with the disclosed antagonists, for example.

Pharmaceutically acceptable carriers are physiologically acceptable to the administered patient and retain the therapeutic properties of the antibodies or peptides with which it is administered. Pharmaceutically-acceptable carriers and their formulations are and generally described in, for example, Remington' pharmaceutical Sciences (18^{th} Edition, ed. A. Gennaro, Mack Publishing Co., Easton, Pa. 1990). One exemplary pharmaceutical carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antibodies or peptides from the administration site of one organ, or portion of the body, to another organ, or portion of the body. Each carrier must be “acceptable” in the sense of being compatible with the other ingredients of the formulation and not injurious to the patient. Nor should a pharmaceutically acceptable carrier alter the specific activity of the antagonists. Exemplary carriers and excipients have been provided elsewhere herein.

In one aspect, the present invention provides pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. The pharmaceutical compositions and formulations include an amount of an invention compound, for example, an effective amount of an antagonist of the invention, and a pharmaceutically or physiologically acceptable carrier.

Pharmaceutical compositions can be formulated to be compatible with a particular route of administration, systemic or local. Thus, pharmaceutical compositions include carriers, diluents, or excipients suitable for administration by various routes.

In a further invention, the compositions of the present invention further comprise a pharmaceutically acceptable additive in order to improve the stability of the antagonist in composition and/or to control the release rate of the composition. Pharmaceutically acceptable additives of the present invention do not alter the specific activity of the subject antagonist. A preferable pharmaceutically acceptable additive is a sugar such as mannitol, sorbitol, glucose, xylitol, trehalose, sorbose, sucrose, galactose, dextran, dextrose, fructose, lactose and mixtures thereof. Pharmaceutically acceptable additives of the present invention can be combined with pharmaceutically acceptable carriers and/or excipients such as dextrose. Alternatively, a preferable pharmaceutically acceptable additive is a surfactant such as polysorbate 20 or polysorbate 80 to increase stability of the peptide and decrease gelling of the pharmaceutical solution. The surfactant can be added to the composition in an amount of 0.01% to 5% of the solution. Addition of such pharmaceutically acceptable additives increases the stability and half-life of the composition in storage.

Formulations or enteral (oral) administration can be contained in a tablet (coated or uncoated), capsule (hard or soft), microsphere, emulsion, powder, granule, crystal, suspension, syrup or elixir. Conventional nontoxic solid carriers which include, for example, pharmaceutical grades of mannitol, lactose, starch, magnesium stearate, sodium saccharin, talcum, cellulose, glucose, sucrose, magnesium carbonate, can be used to prepare solid formulations. Supplementary active compounds (e.g., preservatives, antibacterial, antiviral and antifungal agents) can also be incorporated into the formulations. A liquid formulation can also be used for enteral administration. The carrier can be selected from various oils including petroleum, animal, vegetable or synthetic, for example, peanut oil, soybean oil, mineral oil, sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Pharmaceutical compositions for enteral, parenteral, or transmucosal delivery include, for example, water, saline, phosphate buffered saline, Hank's solution, Ringer's solution, dextrose/saline, and glucose solutions. The formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65 75; Warren (1997) J. Neurol. Sci. 152:31 38; and Tonegawa (1997) J. Exp. Med. 186:507 515. The parenteral preparation can be enclosed in ampules, disposable syringes or multiple dose vials made of glass or plastic.

Pharmaceutical compositions for intradermal or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Pharmaceutical compositions for injection include aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride may be included in the composition. The resulting solutions can be packaged for use as is, or lyophilized; the lyophilized preparation can later be combined with a sterile solution prior to administration.

Pharmaceutically acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacol. 48:119 135; and U.S. Pat. No. 5,391,377, describing lipid compositions for oral delivery of therapeutic agents).

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be through nasal sprays or suppositories (see, e.g., Sayani (1996) “Systemic delivery of peptides and proteins across absorptive mucosae” Crit. Rev. Ther. Drug Carrier Syst. 13:85 184). For transdermal administration, the active compound can be formulated into ointments, salves, gels, or creams as generally known in the art. Transdermal delivery systems can also be achieved using patches.

For inhalation delivery, the pharmaceutical formulation can be administered in the form of an aerosol or mist. For aerosol administration, the formulation can be supplied in finely divided form along with a surfactant and propellant. In another embodiment, the device for delivering the formulation to respiratory tissue is in which the formulation vaporizes. Other delivery systems known in the art include dry powder aerosols, liquid delivery systems, inhalers, air jet nebulizers and propellant systems (see, e.g., Patton (1998) Biotechniques 16:141 143; Dura Pharmaceuticals, San Diego, Calif.; Aradigm, Hayward, Calif.; Aerogen, Santa Clara, Calif.; and Inhale Therapeutic Systems, San Carlos, Calif.).

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as pharmaceutically acceptable carriers. These can be prepared according to methods known in the art, for example, as described in U.S. Pat. Nos. 4,235,871; 4,501,728; 4,522,811; 4,837,028; 6,110,490; 6,096,716; 5,283,185; 5,279,833; Akimaru (1995) Cytokines Mol. Ther. 1:197 210; Alving (1995) Immunol. Rev. 145: 5 31; and Szoka (1980) Ann. Rev. Biophys. Bioeng. 9:467). Biodegradable microspheres or capsules or other biodegradable polymer configurations capable of sustained delivery of small molecules including peptides are known in the art (see, e.g., Putney (1998) Nat. Biotechnol. 16:153 157). Compounds of the invention can be incorporated within micelles (see, e.g., Suntres (1994) J. Pharm. Pharmacol. 46:23 28; Woodle (1992) Pharm. Res. 9:260 265). Antagonists can be attached to the surface of the lipid monolayer or bilayer. For example, antagonists can be attached to hydrazide-PEG-(distearoylphosphatidy-1) ethanolamine-containing liposomes (see, e.g., Zalipsky (1995) Bioconjug. Chem. 6: 705 708). Alternatively, any form of lipid membrane, such as a planar lipid membrane or the cell membrane of an intact cell, e.g., a red blood cell, can be used. Liposomal and lipid-containing formulations can be delivered by any means, including, for example, intravenous, transdermal (see, e.g., Vutla (1996) J. Pharm. Sci. 85:5 8), transmucosal, or oral administration.

Compositions of the present invention can be combined with other therapeutic moieties or imaging/diagnostic moieties as provided herein. Therapeutic moieties and/or imaging moieties can be provided as a separate composition, or as a conjugated moiety. Linkers can be included for conjugated moieties as needed and have been described elsewhere herein.

Medicaments

Pharmaceutical compositions contemplated by the present invention have been described above. In one embodiment of the present invention, the pharmaceutical compositions are formulated to be free of pyrogens such that they are acceptable for administration to human patients. Testing pharmaceutical compositions for pyrogens and preparing pharmaceutical compositions free of pyrogens are well understood to one of ordinary skill in the art.

One embodiment of the present invention contemplates the use of any of the pharmaceutical compositions of the present invention to make a medicament for treating a disorder of the present invention. Medicaments can be formulated based on the physical characteristics of the patient/subject needing treatment, and can be formulated in single or multiple formulations based on the stage of the cancerous tissue. Medicaments of the present invention can be packaged in a suitable pharmaceutical package with appropriate labels for the distribution to hospitals and clinics wherein the label is for the indication of treating a disorder as described herein in a subject. Medicaments can be packaged as a single or multiple units. Instructions for the dosage and administration of the pharmaceutical compositions of the present invention can be included with the pharmaceutical packages.

V. Compositions for Induction of Immune Responses

In another set of embodiments, compositions (e.g., pharmaceuticals) are provided for immunization (vaccination) of a subject to induce an immune response against a denatured ECM component. An immune response can be a humoral immune response or a cell mediated immune response including, but not limited to, an antibody response, a T helper response and/or a cytotoxic T lymphocyte (CTL) response. These pharmaceutical preparations include a pharmaceutically acceptable carrier and a preparation effective to immunize against a denatured ECM component. The preparation is capable of eliciting an antibody response, which antibodies are capable of binding to denatured collagen at the site of a tumor or metastasis and inhibit angiogenesis or tumor development. Alternatively, the preparation is capable of binding to a T cell receptor and initiating a T helper response and/or a cytotoxic T lymphocyte (CTL) response. The preparation can be linked to an immunogenic moiety to increase the likelihood that an immune response will be initiated. Dosage, safety and efficacy of such preparations can be determined in vitro and tested in appropriate in vivo animal models (e.g., rodent and primate) prior to administering the preparation to a human. Vaccines (compositions for immunization) may be made in any convenient manner by one of ordinary skill in the art.

Immunotherapies involve one or more components of the immune system to trigger a complex cascade of biological reactions focused on eliminating a foreign molecule from the host. The immune system consists of a wide range of distinct cell types, the most important of which are the lymphocytes. Lymphocytes determine the specificity of immunity, and it is their response that orchestrates the effector limbs of the immune system. Lymphocytes differ from each other not only in the specificity of their receptors, but also in their functions. One class of lymphocytes, B cells, is a precursor of antibody-secreting cells, and function as mediators of the humoral immune response. Another class of lymphocytes, T cells, expresses important regulatory functions, and mediates cellular immune responses.

Cancer immunotherapy is based on the principle of inducing the immune system to recognize and eliminate neoplastic cells. The key elements in any immunotherapy is inducing the host immune system to first recognize a molecule as an unwanted target, and then inducing the system to initiate a response against that molecule. In healthy hosts, the immune system recognizes surface features of a molecule that is not a normal constituent of the host (i.e., is “foreign” to the host). Once the recognition function occurs, the host must then direct a response against that particular foreign molecule.

Both the recognition and the response elements of the immune system involve a highly complex cascade of biological reactions. In most immunologically based disorders, at least one of the steps in the recognition phase, or at least one of the steps in the response phase, can be modulated or disrupted. Virtually any disruption in either of these complex pathways leads to reduced response or to no response. The inability of the immune system to destroy a growing tumor has been attributed, among other factors, to the presence of tumor-associated antigens (TAA) that induce immunological tolerance and/or immunosuppression. For example, in some kinds of cancer, the cancer itself tricks the host into recognizing a foreign cancer cell as a normal constituent, thus disrupting the recognition phase of the immune system. The immunological approach to cancer therapy involves modification of the host-tumor relationship so that the immune system is induced or amplifies its response to the TAAs. If successful, inducing or amplifying the immune system can lead to tumor regression, tumor rejection, reduced or no tumor growth and occasionally, to tumor cure.

One of the host system's mechanisms for combating a foreign molecule is the humoral response, the production of an antibody against a specific foreign molecule (called an antigen). Typically, the antibody's ability to bind the antigen is based on highly complementary structures.

A particular B or T cell binds to a very specific region of the antigen, called an antigenic determinant or epitope. Thus antigens are molecules that bear one or more epitopes which may be recognized by specific receptors in an immune system, a property called antigenicity.

Immunogenicity is the property of stimulating the immune system to generate a specific response. Thus, all immunogens are antigens, but not vice-versa. Although an immune system may recognize an antigen, it does not respond to the antigen unless the antigen is also immunogenic.

An immune response to a particular antigen is greatly influenced by the structure and activity of the antigen itself, as well as other factors. In some cases, the immune system is not able to generate an immune response to a particular antigen, a condition that is called tolerance.

Immunogenicity is promoted by several factors including foreign, non-human origin, higher molecular weight, greater molecular complexity, tertiary structures, post-translational modifications, the proper antigen dose range, the route of administration, the age of the host, and the genetic composition of the host.

As noted above, antigens may have one or more epitopes or binding sites that are recognized by specific receptors of the immune system. Epitopes may be formed by the primary structure of a molecule (called a sequential epitope), or may be formed by portions of the molecule separate from the primary structure that juxtapose in the secondary or tertiary structure of the molecule (called a conformational or discontinuous epitope). Some epitopes are hidden in the three dimensional structure of the native antigen, and become immunogenic only after a conformational change in the antigen provides access to the epitope by the specific receptors of the immune system. An antigen which is hidden in a tight tissue structure in normal tissue could be accessible in a less tight tissue structure associated with pathological conditions like e.g. cancer. This is an important feature and function in the ability of a therapeutic reagent to initiate recognition and response to an antigen, to induce both a cellular and humoral response to the antigen.

One of the responses generated by the immune system, a humoral response, involves the production of antibodies. Idiotypic determinants, or idiotopes, are markers for the V region of an antibody, a relatively large region that may include several idiotopes each capable of interacting with a different antibody. The set of idiotopes expressed on a single antibody V region constitutes the antibody idiotype. An antibody (Ab1) whose antigen combining site (paratope) interacts with an antigenic determinant on another antibody V region (idiotope) is called an anti-idiotypic antibody (Ab2). Thus, an antibody includes an antigen binding site, and may include one or more antibody binding sites. There are two types of anti-idiotypic antibodies, sometimes called Ab2α and Ab2β. In one type of anti-idiotype antibody (Ab2β), the combining site perfectly mimics the structure of the antigen epitope recognized by the Ab1 antibody. The network theory also suggests that some of these secondary antibodies (Ab2) will have a binding site that is the complement of the complement of the original antigen and thus will reproduce the “internal image” of the original antigen. In other words, an anti-idiotypic antibody may be a surrogate antigen. By definition, the antigen and this type of anti-idiotype antibody compete for the same binding site on Ab1, and the antigen inhibits the interaction between Ab1 and the anti-idiotypic antibody. The phenomenon of producing an anti-idiotypic antibody having the internal image of the antigen may permit the use of antibodies to replace the antigen as an immunogen.

The second type of anti-idiotype, Ab2α, binds to an idiotope of Ab1 that is distinct from the antigen binding site, and therefore may be characterized in terms of the antigen's inability to prevent the binding of the anti-idiotype to Ab1. For this type of anti-idiotype, Ab1 can bind to both the antigen and the anti-idiotypic antibody.

These various interactions based on idiotypic determinants is called the idiotypic network and is based on the immunogenicity of the variable regions of immunoglobulin molecules (Ab1) which stimulate the immune system to generate anti-idiotypic antibodies (Ab2), some of which mimic antigenic epitopes (“internal image”) of the original antigen. The presence of internal image antibodies (Ab2) in the circulation can in turn induce the production of anti-anti-idiotypic antibodies (Ab3), some of which include structures that react with the original antigen.

In addition to a humoral response, the immune system may also generate a cellular response mediated by activated T-cells. There are a number of intercellular signals important to T cell activation. Under normal circumstances an antigen degrades or is cleaved to form antigen fragments or peptides. Presentation of antigen fragments to T-cells is the principal function of MHC molecules, and the cells that carry out this function are called antigen-presenting cells (APC: including but not limited to dendritic cells, macrophages, and B cells).

In addition to generating a humoral response, Ab1 and Ab2 have been shown to induce a cellular immune response characterized by proliferative lymphocytes (helper and suppressor lymphocytes), as well as cytotoxic lymphocytes. Therefore, according to the idiotypic network theory, the injection of anti-denatured collagen antibody should result in the induction of a specific cellular and humoral immune response against the denatured collagen molecule. The concept that anti-idiotypic antibodies function as immunogens has been shown by successful immunization against tumoral, bacterial, viral and parasitic antigens in animal models. Generating Ab2 is an indicator of the existence of a robust immune response that inherently reflects the induction of immune system pathways.

The capture and processing of an antigen by APCs is essential for the induction of a specific immune response. The three major APCs are dendritic cells, macrophages and B-lymphocytes; dendritic cells are the most efficient.

A traditional approach to cancer immunotherapy has been to administer anti-tumor antibodies, i.e., antibodies which recognize an epitope on a tumor cell, to patients. However, the development of the network theory led investigators to suggest the direct administration of exogenously produced anti-idiotype antibodies, that is, antibodies raised against the idiotype of an anti-tumor antibody. Such an approach is disclosed in U.S. Pat. No. 5,053,224 (Koprowski, et al.). Koprowski teaches that the patient's body will produce anti-antibodies that will not only recognize these anti-idiotype antibodies, but also the original tumor epitope.

Two therapeutic applications arose from the network theory: 1) administer Ab1 which acts as an antigen inducing Ab2 production by the host; and 2) administer Ab2 which functionally imitates a tumor antigen.

Support for both applications can be found in the art. For the first approach, active immunization of ovarian cancer patients with repeated intravenous applications of the F(ab′)₂ fragments of the monoclonal antibody OC125 induced remarkable anti-idiotypic antibody (Ab2) responses in some of the patients. Preliminary results suggested that patients with high Ab2 serum concentrations had better survival rates compared to those where low or no Ab2 serum levels were detected. See Wagner, U. et al., “Clinical Course of Patients with Ovarian Carcinomas After Induction of Anti-idiotypic Antibodies Against a Tumor-Associated Antigen,” Tumor Diagnostik & Therapie, 11:1-4, (1990).

For the second approach, human anti-idiotypic monoclonal antibodies (Ab2) have been shown to induce anti-tumor cellular responses in animals and appear to prolong survival in patients with metastatic colorectal cancer. See Durrant, L. G. et al., “Enhanced Cell-Mediated Tumor Killing in Patients Immunized with Human Monoclonal Anti-Idiotypic Antibody 105AD7,” Cancer Research, 54:4837-4840 (1994). The use of anti-idiotypic antibodies (Ab2) for immunotherapy of cancer is also reviewed by Bhattacharya-Chatterje et al.; Cancer Immunol. Immunother. 38:75-82 (1994).

The induction of a specific cellular immune response upon immunization of the host with either an Ab1 or Ab2 antibody has been demonstrated in a number of studies. Of note is the generation through this mechanism of specific cytotoxic T lymphocyte (CTL) responses in ovarian cancer patients, melanoma patients, myeloma patients, and non-Hodgkin's lymphoma patients (Nelson et al., Blood 88(2):580-9 (1996); Madiyalakan et al., Hybridoma 16(1):41-5 (1997); Osterborg et al., Blood 91(7):2459-66 (1998); and Pride et al., Clinical Cancer Research 4:2363 (1998)).

It is therefore expected that the immunization of patients having diseases associated with the denatured collagen peptides provided herein (e.g., angiogenesis, neovascularization, cancer, etc.) with antibodies that preferentially bind to the denatured collagen peptides (i.e., Ab1), or antibodies that mimic the denatured collagen peptides (i.e., Ab2) may also induce a specific and protective CTL immune response against denatured collagen.

In the present application, the extracellular matrix component peptides (e.g., denatured collagen peptides) can be administered to a non-human animal (e.g., a mouse) in order to generate monoclonal antibodies that preferentially bind to the peptides (Ab1), which in turn, initiate the anti-idiotypic network (Ab1→Ab2→Ab3) by inducing Ab2 antibodies that bind the variable region of the Ab1, thereby mimicking the antigen, followed by induction of Ab3 antibodies that specifically bind to the original antigen. These Ab1 antibodies and Ab2 antibodies can be isolated and used for therapeutic purposes. The Ab1 antibodies can, optionally, be humanized.

The peptides as provided herein for inducing an immune response include, but are not limited to, peptides consisting essentially of, or consisting of, a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), where P is hydroxyproline and a pharmaceutically acceptable carrier or excipient. The peptides as provided herein for inducing an immune response also include, but are not limited to, peptides consisting essentially of, or consisting of, a sequence of amino acids set forth as GPPGPP (SEQ ID NO: 81) wherein one or more proline residues is hydroxyproline. The peptide of denatured collagen can be a peptide (or variant thereof), a peptidomimetic (or variant thereof), a linear or a cyclic form thereof, or a pharmaceutically acceptable salt thereof.

Provided herein is a method of inducing an immune response in a patient (human or non-human) by administering to the patient a pharmaceutical composition of any of the peptides provided herein. The immune response can be a humoral immune response or a cellular immune response. When the peptides of the present invention are to be used to induce an immune response, the composition of the peptide is an immunogenic composition that can stimulate a humoral and/or a cell-mediated immune response. The peptide can be a portion of an extracellular matrix component, such as, but not limited to, denatured collagen. Provided herein is method of inducing an immune response and/or the anti-idiotypic network in a subject by administering any of the peptides described herein.

The antagonists for inducing an immune response in a patient are those that bind the a peptide such as, for example, a peptides consisting essentially of, or consisting of, a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), where P is hydroxyproline and a pharmaceutically acceptable carrier or excipient. The antagonists for inducing an immune response in a patient are those that bind the a peptide such as, for example, a peptides consisting essentially of, or consisting of, a sequence of amino acids set forth as GPPGPP (SEQ ID NO: 81) wherein one or more proline residues is hydroxyproline. The peptide of denatured collagen can be a peptide (or variant thereof), a peptidomimetic (or variant thereof), a linear or a cyclic form thereof, or a pharmaceutically acceptable salt thereof.

Provided herein is a method of inducing an immune response in a patient (human or non-human) by administering to the patient a pharmaceutical composition of an antagonist that preferentially binds to an extracellular matrix component as provided herein. The antagonist can, for example, preferentially bind to an extracellular matrix component including, but not limited to, denatured collagen fragments. The antagonists for inducing an immune response in a patient are those that bind the a peptide such as, for example, a peptides consisting essentially of, or consisting of, a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), where P is hydroxyproline. The antagonists for inducing an immune response in a patient are those that bind the a peptide such as, for example, a peptides consisting essentially of, or consisting of, a sequence of amino acids set forth as GPPGPP (SEQ ID NO: 81) wherein one or more proline residues is hydroxyproline. The peptide of denatured collagen can be a peptide (or variant thereof), a peptidomimetic (or variant thereof), a linear or a cyclic form thereof, or a pharmaceutically acceptable salt thereof. The binding site to which the antagonist binds can be a continuous or conformation/dis-continuous epitope. Thus, the antagonist would block the interaction of the extracellular matrix component with its natural ligand. In one non-limiting example, the antagonist preferentially binds to a peptide of denatured collagen and inhibits binding of an integrin to the denatured collagen. The antagonist can be, for example, an antibody.

Provided herein is a method of inducing an immune response in a patient (human or non-human) by administering to the patient a pharmaceutical composition of an antibody that preferentially binds to a denatured extracellular matrix component, where the pharmaceutical composition is an antibody or fragment thereof that induces an effective host immune response against the binding site preferentially bound by said antibody or fragment thereof. The antibody can, for example, preferentially bind to a denatured extracellular matrix component including, but not limited to, denatured collagen fragments. The binding site to which the antibody binds can be a continuous or conformation/dis-continuous epitope. Thus, the antibody would block the interaction of the extracellular matrix component with its natural ligand. In one non-limiting example, the Ab1 antibody preferentially binds to a peptide of denatured collagen and inhibits binding of an integrin to the denatured collagen. In a further embodiment, the Ab1 is humanized prior to its administration to a human patient.

Provided herein is a method of inducing an immune response in a patient (human or non-human) by administering to the patient a pharmaceutical composition of an antibody, where the pharmaceutical composition is an anti-human antibody or fragment thereof that induces an effective host immune response against the variable region binding site of the antibody or fragment thereof. The antibody can be, for example, an anti-idiotypic (Ab2) antibody, which Ab2 antibody mimics a denatured extracellular matrix component. Ab2 antibodies can mimic portions of denatured extracellular matrix components that are shed in angiogenesis-dependent diseases or disorders including, but not limited to, denatured collagen peptides from tumors. The Ab2 antibodies would induce production of an anti-anti-idiotype antibody (Ab3) in vivo. Thus, an effective host immune response is induced against the binding site of said antibody or fragment thereof.

In one embodiment, the immune response is induced against a denatured extracellular matrix component by administering to the patient a pharmaceutical composition, where the pharmaceutical composition is an anti-human antibody or fragment thereof that induces an effective host immune response against the binding site of said antibody or fragment thereof. The binding site of said antibody or fragment thereof mimics the denatured extracellular matrix component and acts to stimulate the anti-idiotypic network. The immune response can be a humoral response or a cell-mediated immune response.

VI. Pharmaceutical Packages and Kits

One embodiment of the present application includes a pharmaceutical package or kit useful for the methods provided herein. One embodiment of such pharmaceutical packages or kits includes preparations (compositions) of the antagonists as provided herein.

Pharmaceutical packages and kits can additionally include an excipient, a carrier, a buffering agent, a preservative or a stabilizing agent in a pharmaceutical formulation. Each component of the kit can be enclosed within an individual container and all of the various containers can be within a single package. Invention kits can be designed for room temperature or cold storage.

Additionally, the preparations can contain stabilizers to increase the shelf-life of the kits and include, for example, bovine serum albumin (BSA) or other known conventional stabilizers. Where the compositions are lyophilized, the kit can contain further preparations of solutions to reconstitute the preparations. Acceptable solutions are well known in the art and include, for example, pharmaceutically acceptable phosphate buffered saline (PBS).

Additionally, the pharmaceutical packages or kits provided herein can further include any of the other moieties provided herein such as, for example, a chemotherapeutic agent.

Pharmaceutical packages and kits of the present invention can further include the components for an assay provided herein, such as, for example, an ELISA assay. Samples to be tested in this application include, for example, blood, plasma, and tissue sections and secretions, urine, lymph, and products thereof. Alternatively, preparations of the kits are used in immunoassays, such as immunohistochemistry to test patient tissue biopsy sections. Pharmaceutical packages and kits of the present invention can further include the components for collection of a sample.

Pharmaceutical packages and kits of the present invention can further include a label specifying, for example, a product description, mode of administration and indication of treatment. Pharmaceutical packages provided herein can include any of the compositions or vaccines as described herein. The pharmaceutical package can further include a label for inhibiting angiogenesis, inhibiting an angiogenesis-dependent disease or disorder, inhibiting tumor development, inducing a host immune response, blocking binding of an integrin to a denatured ECM component, treating a cell-proliferative disorder, treating a cancer or a metastasis, treating diabetic retinopathy, treating macular degeneration or treating neovascular glaucoma, for example.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions. Kits of the invention therefore can additionally include labels or instructions for using the kit components in any method of the invention. A kit can include an invention compound in a pack, or dispenser together with instructions for administering the compound in a method of the invention.

Instructions can include instructions for practicing any of the methods of the invention described herein including treatment, detection, monitoring or diagnostic methods. Instructions may additionally include indications of a satisfactory clinical endpoint or any adverse symptoms that may occur, or additional information required by regulatory agencies such as the Food and Drug Administration for use on a human subject.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM, IC tip and hybrids of these such as magnetic/optical storage media.

The compositions of the kit of the present invention can be formulated in single or multiple units for either a single test or multiple tests.

In preferred embodiments, the preparations of the kit are free of pyrogens. Methods for testing for the presence of, and/or specific levels of, pyrogens is routine in the art and kits are commercially available for such purpose.

VII. Methods of Use

Pharmaceutical compositions of the present invention are administered in a therapeutically effective amount which are effective for producing some desired therapeutic effect by inhibiting angiogenesis, inducing an immune response at a site of angiogenesis thereby causing a tumor-specific killing of tumor cells in a patient and thereby blocking the biological consequences of that pathway in the treated cells eliminating the tumor cell or preventing it from proliferating, at a reasonable benefit/risk ratio applicable to any medical treatment. For the administration of the present pharmaceutical compositions to human patients, the pharmaceutical compositions of the present invention can be formulated by methodology known by one of ordinary skill in the art to be substantially free of pyrogens.

An effective immune response of the present invention is achieved when the patient experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, etc. Overall survival is also measured in months to years. The patient's symptoms may remain static, and the tumor burden may not increase.

Humanized anti-denatured collagen D93 antibody has been shown to have anti-angiogenic and anti-tumor growth properties (Pernasetti, F. et al., Int. J. Oncol. 2006, December; 29(6): 1371-1379). IgG₁k antibody D93 was generated by humanization and affinity maturation using the variable domains of the murine IgM antibody HUI77, which has been shown to exert anti-angiogenic activity in vivo. The binding specificity of D93 for denatured collagen was retained during the humanization process, and the antibody was shown to bind to epitopes on multiple collagen types, including type I, II, III, IV and V, and across diverse species (Pernasetti, F. et al., Int. J. Oncol. 2006, December; 29(6): 1371-1379).

In one preferred embodiment, pharmaceutical compositions of the present invention can be administered to a patient by any convenient route, including, for example, subcutaneous, intravenous, intra-arterial, intraperitoneal, or intramuscular injection.

A physician or veterinarian having ordinary skill in the art can readily determine and prescribe the effective amount (ED50) of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

A. Methods of Inhibiting Angiogenesis and/or an Angiogenesis-Dependent Disease or Disorder

One aspect of the present invention provides a method of preventing, inhibiting and/or treating angiogenesis, comprising contacting a cell or tissue with a therapeutically effective amount of any of the pharmaceutical compositions provided herein.

In one aspect, the present invention provides a method of preventing, inhibiting and/or treating an angiogenesis-dependent disease or disorder in a patient (subject) comprising administering any of the pharmaceutical compositions provided herein to a patient suffering from the angiogenesis-dependent disease or disorder. The angiogenesis-dependent disease or disorder can be a cell proliferative disorder (e.g., a cancer or a metastasis), diabetic retinopathy, macular degeneration or neovascular glaucoma. Inhibiting angiogenesis using the methods provided herein can alleviate symptoms associated with the angiogenesis-dependent disease or disorder.

Angiogenesis-dependent diseases and disorders to be prevented, inhibited or treated include, but are not limited to ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and hemorrhagic teleangiectasia; myocardial angiogenesis; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

Where the angiogenesis-dependent disease or disorder is a cell proliferative disorder (e.g., a cancer or a metastasis), inhibiting in angiogenesis can result in decreased tumor size or prevention of tumor progression. Cancers for treatment using methods herein include, but are not limited to a solid tumor, a metastasis, a cancer, a melanoma, a skin cancer, a breast cancer, a hemangioma or angiofibroma and the like cancer. Exemplary solid tumors can be solid tumors of a tissue or organ such as, for example, skin, melanoma, lung, pancreas, breast, ovary, colon, rectum, stomach, thyroid, laryngeal, ovarian, prostate, colorectal, head, neck, eye, mouth, throat, esophagus, chest, bone, testicular, lymphoid, marrow, bone, sarcoma, renal, sweat gland, liver, kidney, brain, and the like tissues.

In one embodiment provided herein is a method of inhibiting angiogenesis by administering a pharmaceutical composition of an antagonist of a denatured ECM component, e.g., denatured collagen. An antagonist can bind to a denatured ECM component, thereby blocking binding of an integrin to the denatured ECM component, and inhibiting angiogenesis as a result of the blocked interaction. In one non-limiting example, a patient is administered a composition of antibodies or functional fragments thereof that bind, e.g., denatured collagen. Exemplary antibodies or functional fragments thereof are those that preferentially bind to a binding site having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline. Other exemplary antibodies or functional fragments thereof are those that preferentially bind to a binding site having an amino acid sequence set forth as GPPGPP (SEQ ID NO: 81) wherein one or more proline residues is hydroxyproline. Fragments of other ECM components and methods of making antibodies to those fragments have been described elsewhere herein. Antibody antagonists and functional fragments thereof can be generated to preferentially bind such peptide sequences and used in the methods herein. Thus, administering an antibody or functional fragment thereof can prevent, inhibit or treat angiogenesis or an angiogenesis-dependent disease or disorder.

Alternatively, in another embodiment provided herein is a method of inhibiting angiogenesis by administering a pharmaceutical composition of an antagonist of a ligand (e.g., an integrin) of a denatured ECM component (e.g., denatured collagen). An antagonist can bind to the ligand, thereby blocking binding of the integrin to the denatured ECM component, and inhibiting angiogenesis as a result of the blocked interaction. In one example, a patient is administered any of the peptide antagonists provided herein. In one non-limiting example, the method includes administering to a patient, for example, an isolated peptide having a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGP Y (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline, in order to prevent, treat or inhibit angiogenesis or an angiogenic-dependent disorder. In one non-limiting example, the method includes administering to a patient, for example, an isolated peptide having a sequence of amino acids set forth as GPPGPP (SEQ ID NO: 81) wherein one or more proline residues is hydroxyproline, in order to prevent, treat or inhibit angiogenesis or an angiogenic-dependent disorder.

The subject being treated can be a mammal such as a human or a non-human. The composition can be administered locally, regionally or systemically by any suitable route provided herein including, but not limited to, subcutaneously, intradermally, intravenously, intra-arterially, intraperitoneally, and intramuscularly. The methods can further include surgical removal of the cancer, administration of one or more chemotherapeutic agents, or a combination thereof to a patient suffering from cancer.

B. Methods of Treating a Cell Proliferative Disorder

A method of preventing, inhibiting or treating a cell proliferative disorder, comprising administering to a patient having, at risk of having, or having, a cell proliferative disorder a therapeutically effective amount of any of the pharmaceutical compositions provided herein. In one embodiment, the cell proliferative disorder is a cancer or metastasis. At least a part of the cells making up the cell proliferative disorder can be located in blood, breast, lung, thyroid, head or neck, eye, brain, lymph, gastrointestinal tract, nasopharynx, genito-urinary tract, bladder, kidney, pancreas, liver, bone, muscle, skin. ovary, colon, rectum, stomach, thyroid, laryngeal, ovary, prostate, mouth, throat, esophagus, chest, bone, testicular, lymphoid, marrow, bone, sarcoma, renal, sweat gland, liver or the like tissues. In one embodiment, the cell proliferative disorder comprises a benign or malignant solid or non-solid tumor. Tumors provided herein can be metastatic or non-metastatic. In one embodiment, the solid tumor is, for example, a sarcoma or carcinoma.

In one aspect provided herein, administration of the antagonists results in an improvement the subject's condition. Such improvement includes, but is not limited to, decreased cell proliferation, decreased numbers of tumor cells, inhibiting increased cell proliferation, inhibiting increases in numbers of cells, increased apoptosis, or decreased survival, of at least a portion of the cells comprising the cell proliferative disorder, reduction in tumor volume, elimination of a tumor, or a combination thereof. In another aspect, administration of the antagonists prevents the subject's condition from worsening and/or prolongs survival of the patient.

The methods provided herein can further include administering an anti-cancer agent or treatment to the patient.

The patient can be a mammal such as a human or a non-human. Compositions can be administered locally, regionally or systemically by any suitable route provided herein including, but not limited to, subcutaneously, intradermally, intravenously, intra-arterially, intraperitoneally, and intramuscularly.

In one aspect, the present invention provides a method of preventing or treating a cancer or a metastasis in a subject by administering any of the pharmaceutical compositions provided herein to a patient suffering from cancer or metastasis. Such a patient can be symptomatic or asymptomatic.

In some cases, administration of the pharmaceutical composition prolongs life of the patient being treated, reduces tumor volume, eliminates a tumor, decreases cell proliferation, increases apoptosis of tumor cells or a combination thereof. Cancers include without limitation, any cancer known to man. Exemplary cancers are those such as a solid tumor, a metastasis, a melanoma, a skin cancer, a breast cancer, a hemangioma or angiofibroma and the like.

If needed, the method can further include surgical removal of the cancer and/or administration of an anti-cancer agent or treatment. Anti-cancer agents have been provided elsewhere herein.

In one aspect, symptoms of the patient suffering from cancer are ameliorated. Amelioration can be manifested as, for example, reduction in pain, reduced tumor size, elimination of tumors, prevention of increases in tumor size or progression or of disease, prevention of formation of metastasis, or inhibition of metastatic growth, or a combination thereof.

In one aspect, administration of any of the antagonists provided herein reduces or eliminates the need for the patient to undergo surgery or treatment with one or more anti-cancer agents or treatments.

The subject being treated can be a mammal such as a human or a non-human. The composition can be administered locally, regionally or systemically by any suitable route provided herein including, but not limited to, subcutaneously, intradermally, intravenously, intra-arterially, intraperitoneally, and intramuscularly.

In one embodiment, provided herein is a method of preventing, inhibiting or treating a cell-proliferative disorder by administering a pharmaceutical composition containing an antagonist of a denatured ECM component, e.g., denatured collagen. An antagonist can bind to a denatured ECM component, thereby blocking binding of an integrin to the denatured ECM component, and preventing, inhibiting or treating a cell-proliferative disorder as a result of the blocked interaction(s). In one non-limiting example, a patient is administered a composition of antibodies or functional fragments thereof that bind, e.g., denatured collagen, and therefore block binding of an integrin to the denatured collagen. The integrin can be, for example, expressed on a cell such as a transformed cell or a tumor cell. Exemplary antibodies or functional fragments thereof to be delivered to a patient to prevent, inhibit or treat a cell-proliferative disorder include, for example, those that preferentially bind to a binding site having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline and the binding sites are fragments of denatured collagen. Exemplary antibodies or functional fragments thereof to be delivered to a patient to prevent, inhibit or treat a cell-proliferative disorder include, for example, those that preferentially bind to a binding site having an amino acid sequence set forth as GPPGPP (SEQ ID NO: 81) wherein one or more proline residues is hydroxyproline and the binding sites are fragments of denatured collagen. Sequences of other denatured ECM components and methods of making antibodies and functional fragments thereof to those sequences have been described elsewhere herein. Antibody antagonists can be generated to such sequences and used in the methods herein. Thus, administering an antibody or functional fragment thereof can prevent, inhibit or treat a cell-proliferative disorder.

Alternatively, in another embodiment provided herein is a method of preventing, inhibiting or treating a cell, proliferative disorder by administering a pharmaceutical composition of an antagonist of a ligand (e.g., an integrin) of a denatured ECM component (e.g., denatured collagen). An antagonist can bind to the integrin, thereby blocking binding of the integrin to the denatured ECM component, and preventing, inhibiting or treating a cell-proliferative disorder as a result of the blocked interactions. In another example, a patient is administered any of the peptides or peptidomimetics, or fragments thereof, that bind an integrin and block binding of the integrin to the denatured ECM component, thereby preventing, inhibiting or treating a cell-proliferative disorder. The integrin can be, for example, expressed on a cell such as a transformed cell or a tumor cell. Exemplary peptides and peptidomimetics are those that bind to the integrin that is responsible for interacting and binding to an amino acid sequence on a denatured ECM component. Antagonists to be delivered to a patient to prevent, inhibit or treat a cell-proliferative disorder include, for example, isolated peptides consisting essentially of a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), where P is hydroxyproline. In another non-limiting embodiment, antagonists to be delivered to a patient to prevent, inhibit or treat a cell-proliferative disorder include, for example, isolated peptides consisting of a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), where P is hydroxyproline. In another non-limiting embodiment, antagonists to be delivered to a patient to prevent, inhibit or treat a cell-proliferative disorder include, for example, isolated peptides consisting essentially of a sequence of amino acids set forth as GPPGPP (SEQ ID NO: 81) wherein one or more proline residues is hydroxyproline. In yet another non-limiting embodiment, antagonists to be delivered to a patient to prevent, inhibit or treat a cell-proliferative disorder include, for example, isolated peptides consisting essentially of a sequence of amino acids set forth as GPPGPP (SEQ ID NO: 81) wherein one or more proline residues is hydroxyproline. Thus, administering a peptide or peptidomimetic as described herein can prevent, inhibit or treat a cell-proliferative disorder.

C. Method of Inducing an Immune Response/Vaccination

In one aspect, the present invention provides a method of inducing an immune response in a patient by administering to a patient any of the pharmaceutical compositions of provided herein, where the compound of the composition induces an effective host immune response against an amino acid sequence recognized by the composition.

In one non-limiting example, the composition contains an antibody or functional fragment thereof that specifically binds to a denatured extracellular matrix (ECM) component, thereby blocking binding of a natural ligand (e.g., integrin) to the denatured ECM component. In one embodiment, the extracellular matrix component can be cryptic collagen epitopes, cryptic laminin epitopes, fibronectin, vitronectin, fibrinogen, thrombospondin, osteopontin, tenascin, and vWF. In one embodiment, the natural ligand is an integrin including, but not limited to, α1β1, α2β1, α3β1, α5β1, α6β1, α7β1, α8β1, α9β1, α10β1, α11β1, αvβ1, αvβ3, αvβ5, αvβ8, αvβ6, αvβ8 and α6β4. The patient being treated can be a mammal such as a human or a non-human. The composition can be administered locally, regionally or systemically by any suitable route provided herein including, but not limited to, subcutaneously, intradermally, intravenously, intra-arterially, intraperitoneally, and intramuscularly.

In another non-limiting example, the composition contains a peptide or peptidomimetic as provided elsewhere herein, which peptides and peptidomimetics mimic fragments of denatured ECM components, thereby blocking binding of an integrin to the denatured ECM component.

In one embodiment, a humoral immune response (i.e., an antibody response) is raised against a peptide in vivo. Such an immune response can be focused to an area of angiogenesis as a result of localization of the peptides to, for example, integrins near ECM components at sites of angiogenesis. Thus, an immune response can be “focused.” Antibodies that are induced in response to the peptide can then bind to the ECM component from which the peptide was derived, thereby inhibiting angiogenesis. Alternately, or in addition, a T helper response can be initiated resulting in the productions of cytokines, e.g., IFN-γ, that have been shown to play a role in treating cancers and metastases. In another embodiment, a cell-mediated immune response is initiated and the CTLs kill tumor cells expressing the integrin.

In one embodiment, the extracellular matrix component can be cryptic collagen epitopes, cryptic laminin epitopes, fibronectin, vitronectin, fibrinogen, thrombospondin, osteopontin, tenascin, and vWF. In one embodiment, the peptide is a fragment of an ECM component. The integrin can be, for example, α1β1, α2β1, α3β1, α5β1, α6β1, α7β1, α8β1 α9β1, α10β1, α11β1, αvβ1, αvβ3, αvβ5, αvβ8, αvβ6, αvβ8 and α6β4. The subject being treated can be a mammal such as a human or a non-human. The composition can be administered locally, regionally or systemically by any suitable route provided herein including, but not limited to, subcutaneously, intradermally, intravenously, intra-arterially, intraperitoneally, and intramuscularly. Inducing an immune response in this manner can have the effect of vaccinating the patient.

In one non-limiting example, a patient is administered a peptide that induces an immune response. Exemplary peptides are those that bind to an integrin that binds to an amino acid sequence on a denatured ECM component. In one embodiment, the peptides binds to an integrin expressed on a tumor cell and blocks the tumor cell from binding to the ECM component. Peptide antagonists to be delivered to a patient to prevent, inhibit or treat a cell-proliferative disorder include, for example, peptides having a sequence of amino acids set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline. Peptide antagonists to be delivered to a patient to prevent, inhibit or treat a cell-proliferative disorder include, for example, peptides having a sequence of amino acids set forth as GPPGPP (SEQ ID NO: 81) wherein one or more proline residues is hydroxyproline. In one non-limiting embodiment, the peptide antagonist consists essentially of, or consists of, an amino acid sequence as described herein. Thus, administering a peptide or peptidomimetic as described herein can be used to induce an immune response (vaccinate) in a patient.

D. Method of Treating Diabetic Retinopathy, Macular Degeneration or Neovascular Glaucoma

In one aspect, the present invention provides a method for treating diabetic retinopathy, macular degeneration or neovascular glaucoma in a patient by administering to the patient a therapeutically effective amount of any of the pharmaceutical compositions provided herein.

In one non-limiting example, a patient is administered an antibody or functional fragment thereof that preferentially binds to a binding site on a denatured ECM component (e.g., denatured collagen). Binding sites on denatured collagen include, but are not limited to the amino acid sequences set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline. Binding sites on denatured collagen also include, but are not limited to the amino acid sequences set forth as GPPGPP (SEQ ID NO: 81) wherein one or more proline residues is hydroxyproline. Binding of the antibody or functional fragment thereof to such binding sites inhibits angiogenesis, thereby treating diabetic retinopathy, macular degeneration or neovascular glaucoma in the patient.

In one non-limiting example, a patient is administered a peptide or peptidomimetic antagonist thereof that binds to a binding site on an integrin. Peptides include, but are not limited to fragments of denatured collagen having an amino acid sequence such as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline. Peptide antagonists to be delivered to a patient to prevent, inhibit or treat a cell-proliferative disorder include, for example, peptides having a sequence of amino acids set forth as GPPGPP (SEQ ID NO: 81) wherein one or more proline residues is hydroxyproline. In one non-limiting embodiment, the peptide antagonist consists essentially of, or consists of, an amino acid sequence as described herein. Binding of the peptide or peptidomimetic to the integrin prevents binding of the integrin to denatured collagen, thereby inhibiting angiogenesis and, therefore, treating diabetic retinopathy, macular degeneration or neovascular glaucoma in a patient having one of these conditions.

The subject being treated can be a mammal such as a human or a non-human. The composition can be administered locally, regionally or systemically by any suitable route provided herein including, but not limited to, subcutaneously, intradermally, intravenously, intra-arterially, intraperitoneally, and intramuscularly.

E. Method of Blocking Binding of a Ligand to an Extracellular Matrix (ECM) Component

In one aspect, the present invention provides a method of blocking binding of a ligand (e.g., an integrin) to a denatured extracellular matrix (ECM) component by administering any of the pharmaceutical compositions provided herein to a patient. The ECM component can be one or more of cryptic collagen epitopes, cryptic laminin epitopes, fibronectin, vitronectin, fibrinogen, thrombospondin, osteopontin, tenascin, vWF, or a combination thereof. The ligand can be an integrin including, but not limited to, α1β1, α2β1, α3β1, α4β1, α5β1, α6β1, α7β1, α8β1, α9β1, α10β1, α11β1, αvβ1, αvβ3, αvβ5, αvβ8, αvβ6, αvβ8 and α6β4. The subject can be a mammal such as a human or a non-human. The composition can be administered locally, regionally or systemically by any suitable route provided herein including, but not limited to, subcutaneously, intradermally, intravenously, intra-arterially, intraperitoneally, and intramuscularly.

In one aspect, the present invention provides a method of blocking binding of a ligand to an extracellular matrix (ECM) component by administering to a patient an antibody or functional fragment thereof that preferentially binds to a binding site on denatured collagen including, but not limited to, a binding site having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline, thereby blocking binding of an integrin to a denatured collagen. In another aspect, the present invention provides a method of blocking binding of a ligand to an extracellular matrix (ECM) component by administering to a patient an antibody or functional fragment thereof that preferentially binds to a binding site on denatured collagen including, but not limited to, a binding site having an amino acid sequence set forth as GPPGPP (SEQ ID NO: 81) wherein one or more proline residues is hydroxyproline.

In another aspect, the present invention provides a method of blocking binding of a ligand (e.g., an integrin) to a denatured extracellular matrix (ECM) component by administering to a patient a peptide or peptidomimetic that binds to a binding site on an integrin. Peptides include, an amino acid sequence such as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline, thereby blocking binding of an integrin to a denatured collagen. Peptides include, an amino acid sequence such as GPPGPP (SEQ ID NO: 81) wherein one or more proline residues is hydroxyproline, thereby blocking binding of an integrin to a denatured collagen. In one non-limiting embodiment, the peptide or peptidomimetic consists essentially of, or consists of, an amino acid sequence as described herein.

F. Methods of Contacting Cells

One aspect of the present invention provides a method, comprising contacting a cell with any of the pharmaceutical compositions provided herein, wherein said contacting inhibits binding of an cell expressing an integrin to an extracellular matrix component. The method encompasses any method of in vitro, ex vivo or in vivo use of the compositions. The cell can be a cultured cell, or is present in a subject (e.g., a human or a non-human). The cell can be, for example, a tumor cell. When the cell is a cultured cell, the composition can be provided by any means used in the art of cell culture. Such methods are well known in the art. When the cell is present in a subject, the composition can be administered locally, regionally or systemically by any suitable route provided herein including, but not limited to, subcutaneously, intradermally, intravenously, intra-arterially, intraperitoneally, and intramuscularly.

In one aspect, the present invention provides for a method of contacting a cell with an antibody or functional fragment thereof as described herein. In one aspect, the present invention provides for a method of contacting a cell with a peptide, peptidomimetic, or variants thereof or pharmaceutically acceptable salts thereof as described herein. The method can include administration of the compounds in a pharmaceutically acceptable carrier to an assay plate in vitro for testing of binding or blocking of the compound to a ligand (e.g., an integrin), ECM component, or denatured ECM component.

Alternatively, the method can include in vivo administration of a peptide or peptidomimetic of the present invention to an animal model in order to generate monoclonal antibodies. Isolated monoclonal antibodies can be used as such or humanized for administration to human patients in any of the methods provided herein.

In a third embodiment, the compounds provided herein can be administered by any usable route to a patient for preventing, inhibiting, or treating any of the diseases or disorders as described herein.

In one embodiment, the method of contacting a cell with an antagonist of the invention includes administering an antibody or functional fragment thereof that preferentially binds to a binding site on a denatured ECM component e.g., denatured collagen including, which binding sites include, but are not limited to, the amino acid sequences set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline, thereby blocking binding of an integrin to denatured collagen. In another embodiment, the method of contacting a cell with an antagonist of the invention includes administering an antibody or functional fragment thereof that preferentially binds to a binding site on a denatured ECM component e.g., denatured collagen including, which binding sites include, but are not limited to, the amino acid sequences set forth as GPPGPP (SEQ ID NO: 81) wherein one or more proline residues is hydroxyproline, thereby blocking binding of an integrin to denatured collagen. An antibody or functional fragment thereof can also be administered that preferentially binds to a binding site on an integrin, which binding blocks an interaction of the integrin with a denatured ECM component.

In another embodiment, the method of contacting a cell with an antagonist of the invention includes administering a peptide or peptidomimetic, or variants thereof, or pharmaceutically acceptable salt thereof that binds to an integrin. Peptides include, but are not limited to, fragments of denatured collagen having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), or variants thereof, where P is hydroxyproline, thereby blocking binding of an integrin to denatured collagen. In another embodiment, the method of contacting a cell with an antagonist of the invention includes administering a peptide or peptidomimetic, or variants thereof, or pharmaceutically acceptable salt thereof that binds to an integrin. Peptides include, but are not limited to, fragments of denatured collagen having an amino acid sequence set forth as GPPGPP (SEQ ID NO: 81) wherein one or more proline residues is hydroxyproline, thereby blocking binding of an integrin to denatured collagen. In one non-limiting embodiment, the peptide or peptidomimetic consists essentially of, or consists of, an amino acid sequence as described herein.

G. Methods of Inducing CDC, ADCC or Opsonization

Various therapies have been directed to augmenting the body's natural immune response to transformed cells. Conventional effector methods include complement dependent cytolysis (“CDC”), antibody dependent cellular cytotoxicity (“ADCC”) and phagocytosis (clearance by reticuloendothelial system after the target cell is coated with immunoglobulin). It is known that in the presence of antibodies, certain effector cells, such as lymphoid cells having surface bound receptors for the Fc regions of antibodies, mediate an antibody dependent cellular cytoxicity (“ADCC”) reaction against target cells. By means of ADCC, these effector cells exert cytolytic activity against such target cells.

Two types of ADCC reactions have been demonstrated in vitro. In classical ADCC reactions, effector cells attach to antibody-coated target cells and subsequently cause cytolysis of the target cells (A. H. Greenberg et al., “Characteristics Of The Effector Cells Mediating Cytotoxicity Against Antibody-Coated Target Cells.” I., Immunology, 21, p. 719 (1975)). This attachment between effector and target cell results from the interaction of the Fc region of the antibody coating the target cell and the Fc receptor of the effector cell. One disadvantage of this type of ADCC reaction is that it may be hampered by circulating antigen-antibody complexes, often associated with various diseases, which compete with the target-cell bound antibody for the Fc receptors of the effector cells (I. C. M. MacLennan, “Competition For Receptors For Immunoglobulin On Cytotoxic Lymphocytes”, Clin. Exp. Immunol., 10, p. 275 (1972)). Due to this drawback of classical ADCC, a second type of ADCC reaction—antibody-directed ADCC—has been proposed. In antibody-directed ADCC, the target-specific antibody is first attached to the effector cell and the resulting complex is then “directed,” via the antibody, to its specific antigen on the target cell surface. Advantageously, antibody-directed ADCC may not be affected by the presence of antigen-antibody complexes circulating in the host system. The interaction between antibodies and effector cells via Fc region/Fc receptor attachment is normally weak. And, in some instances, antibodies do not remain associated with effector cells for a period of time sufficient to permit lysis of target cells. In view of this potential problem, antibodies have been attached to the effector cells using pre-treatment with polyethylene glycol and a mixture of phthalate oils (J. F. Jones and D. M. Segal, “Antibody-Dependent Cell Mediated Cytolysis (ADCC) With Antibody-Coated Effectors: New Methods For Enhancing Antibody Binding And Cytolysis,” J. Immunol., 125, pp. 926-33 (1980)). The applicability of this method for in vivo treatments, however, may be diminished by the toxic effects that any polyethylene glycol and phthalate oil residues on the antibody-effector cell complex may have on the body.

Alternatively, a method has been proposed for enhancing antibody-directed ADCC by adjuvant chemotherapy with cytotoxic drugs (I. R. Mackay et al., “Effect On Natural Killer And Antibody-Dependent Cellular Cytotoxicity Of Adjuvant Cytotoxic Chemotherapy Including Melphalan In Breast Cancer”, Cancer Immunol. Immunother., 16, pp. 98-100 (1983)). Assays for testing for ADCC are well-known in the art, such as for example, U.S. Pat. No. 5,756,097.

Accordingly, the present invention provides antibodies (e.g., D93) that can bind to cells having a role in neovascularization or angiogenesis of that can enhance phagocytosis and killing of the cells and thereby enhance protection in vivo. Also provided are other antibodies and functional fragments thereof that immunoreact, specifically bind to, or preferentially bind to a binding site or epitope to which such antibodies can bind and which have the same effect.

The antibodies of the invention can also be opsonic, or exhibit opsonic activity, for cells having a role in neovascularization or angiogenesis (e.g., transformed cells). As those in the art recognize, “opsonic activity” refers to the ability of an opsonin (generally either an antibody or the serum factor C3b) to bind to an antigen or cell receptor to promote attachment of the antigen or cell receptor to a phagocyte and thereby enhance phagocytosis. Certain cells become extremely attractive to phagocytes such as neutrophils and macrophages when coated with an opsonic antibody and their rate of clearance from the bloodstream is strikingly enhanced. Opsonic activity may be measured in any conventional manner as described, for example, in U.S. Pat. No. 6,610,293.

In one non-limiting embodiment, a peptide antagonist of the present invention serves to initiate the complement cascade, opsonization, or ADCC. For example, a peptide can be systemically administered to a patient, which peptide will bind to its ligand. In one specific example, a peptide of the present invention binds to an integrin on a cell at a site of neovascularization or angiogenesis. Sufficient time is allowed for the peptide to bind to such integrins including, up to 1 hour, up to 2 hrs, up to 3 hrs, up to 4 hrs, up to 5 hrs, up to 6 hrs, and up to 12-24 hours. Subsequent to administration of the peptide, an antibody that preferentially binds to the peptide is systemically administered. Binding of the antibody antagonist to the denatured ECM component can initiate any of the pathways described herein to induce CDC, ADCC, opsonization, or any other form of cell-mediated killing.

In another non-limiting embodiment, a patient having a neovascular disorder or an angiogenesis dependent disorder, or any other disease or disorder associated with breakdown of an ECM component (e.g., collagen) sheds antigens/peptides from the degradation of the ECM components. These antigens/peptides can be “tumor associated antigens.” Such patients can be systemically administered an antibody to the antigen/peptide (e.g., denatured collagen) and can initiate any of the pathways described herein to induce CDC, ADCC, opsonization, or any other form of cell-mediated killing.

H. Combination Therapies

In one aspect, the present invention includes combination therapies comprising administering the compositions provided in combination with any of the other therapeutic moieties provided herein. Such other therapeutic moieties include without limitation, antagonists, chemotherapeutic agents, toxins, etc. as provided elsewhere herein.

In combination therapies provided herein, compositions administered in combination with the compositions of the present invention (e.g., antagonists) can be administered prior to, concurrently with, or subsequent to the compositions of the present invention. Such administrations include, but are not limited to, administration within a week before the antagonist, within a week after the antagonist, on the same day as the antagonist, at the same time as the antagonist or a combination thereof. When multiple doses of the composition of the present invention and/or the combined therapeutic moiety are contemplated, it is understood that doses of each can be empirically determined using known doses and concentrations based on the age, height, weight, health and other physical characteristics using standards of commercially available products.

In some aspects of the present invention, therapies can further comprising surgical removal of a cancer or metastasis.

In one aspect, the present invention provides for a method for treating cancer, including surgical removal of the cancer and concurrent administration of an anti-cancer agent or treatment and the pharmaceutical composition of one or more of any of the antagonists provided herein to a patient suffering from cancer. The anti-cancer agent or treatment can be administered once or multiple times. The patient can mammal such as a human or non-human mammal. The composition can be administered locally, regionally or systemically by any suitable route provided herein including, but not limited to, subcutaneously, intradermally, intravenously, intra-arterially, intraperitoneally, and intramuscularly.

In one non-limiting method for treating cancer provided herein, the method includes surgical removal of the cancer and concurrent administration of an anti-cancer drug or treatment and an antagonist. In one aspect, administration of the antagonist can be, for example, a 20 minute intravenous infusion. In one non-limiting example, the anti-cancer drug or treatment is administered within seven days prior to the administration of the antagonist. Alternatively, the anti-cancer drug or treatment is administered within seven days following the administration of the antagonist. In one embodiment, the anti-cancer drug or treatment is administered every four weeks for six cycles. The method can further include the step of administering the antagonist every twelve weeks for up to two years. In one non-limiting example, the antagonist and anti-cancer drug or treatment are administered at weeks 1, 4, and 8, followed by further administration of the anti-cancer drug or treatment alone at weeks 12 and 16, followed by concurrent administration of the anti-cancer drug or treatment and antagonist at week 20. In another non-limiting example, the concurrent administration of the antagonist and the anti-cancer drug or treatment occurs at week 1, followed by administration of the anti-cancer drug or treatment at week 4, wherein the concurrent administration is repeated for six cycles and followed by administration of the antagonist every twelve weeks for up to two years.

In one non-limiting example of a method for treating cancer in a patient, the method includes surgical removal of the cancer and administration of an antagonist at weeks 1, 3, 5, 7 and 9 followed by concurrent administration of an anti-cancer drug or treatment and an antagonist in a dose week 12. Additionally, the concurrent administration of the anti-cancer drug or treatment and antagonist can be repeated every four weeks for up to 6 cycles. Optionally, the method further includes administering the antagonist every twelve weeks for up to two years. It will be understood that treatment regimens can be combined with monitoring methods provided herein to determine if and when additional doses of antagonists and/or anti-cancer drug or treatment need be administered.

The combination therapy may provide a synergistic and/or beneficial effect or may allow lower doses of a combination to provide a greater margin of safety. The invention encompasses treatment protocols that enhance the prophylactic or therapeutic effect of an antagonist of denatured or proteolyzed collagens using a specific antibody, or a fragment derived from an antibody, compound cross-linked or genetically fused to an antibody, peptide, peptidomimetic or mimetic epitope which binds the antibody, an antibody gene(s) using gene therapies for preventing, managing, treating or ablation of cancer or other diseases. Additionally, such antagonists can be formulated either together or separately and delivered by the same or different route of administration depending on the optimum formulation and bioactive species for a patient with a specific disease. The antagonists provided herein can also be used in diagnostic and/or imaging assays and methods.

In one embodiment, compositions of the present invention can be combined with peptides, antibodies and siRNA, DNA or RNA sequences that encode integrin peptide sequences including, but not limited to, those shown in Table 1. Some the integrin peptides are referenced with names for potential therapeutic and/or diagnostic use, such as Tysabri, Vitaxin and ReoPro. For most therapeutic indications the compounds are administered to inhibit diseases such as cancer and inflammation but may be used to augment host biological responses in certain disease conditions.

TABLE 1
Integrin Peptides, Antibodies and Derivatives

| | Antibodies, | |
|---|---|---|
| | Peptide or | |
| Protein | Mimetics | Reference |
| α1β1 | Anti-VLA1 | J Clin Invest. 2002 December; |
| | | 110(12): 1773-82. |
| α2β1 | antibody | Br J Cancer. 1998 June; |
| | | 77(12): 2274-80. |
| α3β1 | antibody | Exp Cell Res. 1995 July; |
| | | 219(1): 233-42 |
| α4β1 | Anti-VLA-4 | Steinman L. Nat Rev Drug |
| | Natalizumab | Discov. 2005 June; 4(6): |
| | Tysabri | 510-8 |
| α5β1 | Volocixmab | J Biol Chem. 1997 Jul. 11; |
| | Anti-alpha5 | 272(28): 17283-92 |
| α6β1 | Anti-alpha6 | Gastroenterology. 1995 |
| | | February; 108(2): 523-32. |
| | | J Hepatol. 1999 October; |
| | | 31(4): 734-40 |
| α7β1 | antibodies | J Biol Chem. 1996 Oct. |
| | | 11; 271(41): 25598-603 |
| α8β1 | siRNA | Cardiovasc Res. 2005 Mar. 1; |
| | | 65(4): 813-22 |
| α9β1 | antibody | Mol Biol Cell. 2005 February; |
| | | 16(2): 861-70. Epub 2004 Dec. 1 |
| α10β1 | potential all | J Cell Sci. 2005 Mar. 1; |
| | | 118(Pt 5): 929-36. Epub |
| | | 2005 Feb. 15. |
| α11β1 | antibody, | Dev Biol. 2004 Jun. 15; |
| | potential all | 270(2): 427-42. |
| αVβ1 | Anti-CD51 (αV) | J Med Chem. 2005 Feb. 24; |
| | CNTO 95 | 48(4): 1098-106 |
| | (Centocor) Cyclic | |
| | RGD peptide | |
| αVβ3 | LM609, Vitaxin | Ann Rheum Dis. 2002 November; |
| | (Medimmune), | 61 Suppl 2: ii96-9. J Nucl Med. |
| | (18)F-Galacto-RGD | 2005 August; 46(8): 1333-41 |
| αVβ5 | antibody | Eur J Cancer. 2005 May; |
| | | 41(7): 1065-72. |
| αVβ8 | Anti-beta8 | Am J Pathol. 2003 August; |
| | | 163(2): 533-42 |
| αVβ6 | antibodies | Exp Cell Res. 2000 Feb. 25; |
| | | 255(1): 10-7. |
| αVβ8 | RGD peptides | Exp Cell Res. 2003 Dec. 10; |
| | | 291(2): 514-24. |
| α6β4 | antibodies | J Biol Chem. 2005 Mar. 4; |
| | | 280(9): 8004-15. Epub 2004 |
| | | Dec. 3. |
| Substituted | Chemical | U.S. Pat. No.: 6,855,722 |
| Indole | compounds | |
| Integrin | | |
| Antagonists | | |
| Vitronectin | Chemical | U.S. Pat. No.: 6,818,201 |
| Receptor | compounds | |
| Antagonists | | |

In another embodiment, compositions of the present invention can be combined with peptides, antibodies, siRNA, DNA or RNA sequences that encode cell surface peptide sequences including those shown in Table 2. For most therapeutic indications the compounds are administered to inhibit diseases such as cancer and inflammation but may be used to augment host biological responses in certain disease conditions.

TABLE 2
Cell Surface Peptides, Antibodies and Derivatives

| | Antibodies, Peptide | |
|---|---|---|
| Protein | or Mimetics | Reference |
| erbB-2 or neu | Herceptin, | Anticancer Res. 2005 |
| | trastuzumab; peptide | July-August; 25(4): 3061-6 |
| | | Cancer Res. 2005 Aug. 1; |
| | | 65(15): 6891-900 Oncology. |
| | | 2001; 61 Suppl 2: 14-21 |
| CD44 | antibody | Anticancer Res. 2005 |
| | | March-April; 25(2A): 1115-21. |
| LFA-1 | Efalizumab, Raptiva | Ann Pharmacother. 2005 |
| | | September; 39(9): 1476-82. |
| | | Epub 2005 Jul. 5 |
| CD11a | Efalizumab | Am J Clin Dermatol. 2005; |
| | | 6(2): 113-8; |
| CD11b | antibody | J Infect Dis. 2005 May 15; |
| | | 191(10): 1755-60. Epub 2005 |
| | | Apr. 6. |
| CD11c | antibody | Arch Dermatol Res. 1997 |
| | | November; 289(12): 692-7. |
| (GP) IIb/IIIa | ReoPro; abciximab | Chest. 2005 February; |
| receptor | | 127(2 Suppl): 53S-59S |

In another embodiment, compositions of the present invention can be combined with peptides, antibodies, siRNA, DNA or RNA sequences that encode laminin peptide sequences including those shown in Table 3. For most therapeutic indications the compounds are administered to inhibit diseases such as cancer and inflammation but may be used to augment host biological responses.

TABLE 3
Laminin Peptides, Antibodies and Derivatives

| | Antibodies, | |
|---|---|---|
| | Peptide or | |
| Protein | Mimetics | Reference |
| Laminin 5 | Potential | Curr Opin Cell Biol. 2000 |
| | for all | October; 12(5): 554-62. |
| Laminin 1 | Peptide, | Endocr Relat Cancer. 2005 June; |
| | antibody | 12(2): 393-406. Clin Dev |
| | | Immunol. 2005 March; 12(1): 67-73. |
| Laminin 8 | antibody | Breast Cancer Res. 2005; 7(4): |
| | | R411-21. Epub 2005 April |
| Laminin 9 | antibody | Breast Cancer Res. 2005; |
| | | 7(4): R411-21. Epub 2005 April |
| Laminin 3 | antibody | Exp Cell Res. 2000 Sep. 15; |
| | | 259(2): 326-35. |
| Laminin 10 | antibody | Neoplasia. 2005 April; 7(4): 380-9. |
| Laminin 8-11 | antibody | J Cell Biol. 1997 May 5; |
| | | 137(3): 685-701 |

In another embodiment, compositions of the present invention can be combined with peptides, antibodies, siRNA, DNA or RNA sequences that encode other ECM proteins including those shown in Table 4. For most therapeutic indications the compounds are administered to inhibit diseases such as cancer and inflammation but may be used to augment host biological responses in certain disease conditions.

TABLE 4
Other ECM or Secreted Peptides, Antibodies and Derivatives

| | Antibodies, Peptide or | |
|---|---|---|
| Protein | Mimetics | Reference |
| Nidogen | Antibody, | Eur J Biochem. 1987 Jul. 1; |
| | potential for all | 166(1): 11-9. |
| Perlecan | Antibody, | Matrix. 1992 June; 12(3): |
| | potential for all | 221-32 |
| Osteopontins | Antibody, | Gut. 2005 September; 54(9): |
| | potential for all | 1254-62. |
| BMPs | Antibody, | Growth Factors. 2004 |
| | potential for all | December; 22(4): 233-41. |
| Versican | Potential for all | Cell Res. 2005 |
| | | July; 15(7): 483-94 |
| Decorin | Potential for all | Exp Mol Pathol. 2005 |
| | | August; 79(1): 68-73. |
| Biglycan | Potential for all | J Dermatol Sci. 2005 June 29 |
| TSP-1 | Peptide mimetics | J Med Chem. 2005 Apr. 21; |
| | | 48(8): 2838-46. |
| Fibronectin | Antibodies, peptides | Expert Opin Ther Targets. |
| | | 2005 June; 9(3): 491-500. |
| VEGF | Avastin, Macugen, | Expert Opin Biol Ther. 2005 |
| | | July; 5(7): 997-1005 |
| Vitronectin | Antibodies, potential | J Clin Invest. 1990 May; |
| | for all | 85(5): 1372-8 |

In another embodiment, compositions of the present invention can be combined with peptides, antibodies, siRNA, DNA or RNA sequences that encode proteolytic enzymes shown in Table 5. The proteases described in Table 5 cover general classes of enzymes. For most therapeutic indications the compounds are administered to inhibit diseases such as cancer and inflammation but may be used to augment host biological responses in certain disease conditions.

TABLE 5
Enzyme Peptides, Antibodies and Derivatives

| | Antibodies, | |
|---|---|---|
| | Peptide | |
| Protein | or Mimetics | Reference |
| MMPs | Enzyme inhibitors | Curr Cancer Drug Targets. |
| | | 2005 May; 5(3): 203-20 |
| ADAMTS | Enzyme Inhibitors | Biochem J. 2005 Feb. 15; |
| | | 386(Pt 1): 15-27 |
| Cathepsins | Enzyme inhibitors | Neoplasma. 2005; 52(3): |
| (B, D and L) | | 185-92. |
| TIMPs | peptide | Trends Mol Med. 2005 March; |
| | | 11(3): 97-103 Crit Rev |
| | | Oncol Hematol. 2004 March; |
| | | 49(3): 187-98 |
| Caspase | Protein, agonists | Oncology (Williston Park). |
| | | 2004 November; |
| | | 18(13 Suppl 10): 11-20 |
| uPA | Enzyme inhibitor | Neoplasma. 2005; 52(3): |
| | | 185-92. |
| Aggrecan and | Potential for all, | Mol Cell Proteomics. |
| aggrecanase | enzyme inhibitors | 2005 Jun. 21 |

In another embodiment, compositions of the present invention can be combined with peptides, antibodies, siRNA, DNA or RNA sequences that encode other immune stimulants shown in Table 6. Some of the ECM proteins are referenced with names for potential therapeutic and/or diagnostic use, such as Canvaxin and Provenge. For most therapeutic indications the compounds are administered to inhibit diseases such as cancer and inflammation but may be used to augment host biological responses in certain disease conditions.

TABLE 6
Immune Stimulants

| | Antibodies, Peptide | |
|---|---|---|
| Protein | or Mimetics | Reference |
| Canvaxin | Cell-based active | Dev Biol (Basel). |
| | immunotherapy | 2004; 116: 209-17; |
| | | discussion 229-36 |
| Provenge | Peptide loaded APC | Prostate. 2004 Aug. |
| | | 1; 60(3): 197-204. |
| Tumor | Several approaches | J Clin Oncol. 2001 Mar. |
| Vaccines | | 15; 19(6): 1848-54 |

In another embodiment, compositions of the present invention can be combined with peptides, antibodies, siRNA, DNA or RNA sequences that encode other proteins shown in Table 7. Some of the RTK proteins are referenced with names for potential therapeutic and/or diagnostic use, such as Tarceva, Gleevec and Iressa. For most therapeutic indications the compounds are administered to inhibit diseases such as cancer but may be used to augment host biological responses in certain disease conditions.

TABLE 7
Tyrosine Kinase Receptor (RTK) Inhibitors

| | Antibodies, Peptide | |
|---|---|---|
| Protein | or Mimetics | Reference |
| EGFR | Tarceva, Gefitinib | Cancer Invest. 2005; |
| | (Iressa) | 23(4): 296-302 J |
| | | Pharmacol Exp Ther. |
| | | 2005 Jul. 7 |
| EGFR | Gleevec | J Pharmacol Exp Ther. |
| | | 2005 Jul. 7 |
| RTK General | Enzyme inhibitor | J Pharmacol Exp Ther. |
| | | 2005 Jul. 7 |

In another embodiment, the compositions of the present invention can be combined with peptides derived from collagen peptide sequences including those shown in Table 8. Some of the collagen peptides are referenced with names for potential therapeutic and/or diagnostic use, such as Arrestin, Canstatin, etc. For most therapeutic indications the compounds are administered to inhibit diseases such as cancer and inflammation but may be used to augment host biological responses in certain disease conditions.

TABLE 8
Collagen-Derived Peptides

| Collagen | | |
|---|---|---|
| Peptide | Parent Protein | Reference |
| Human | NC1 domanin, | O'Reilly, MS et al. Cell |
| Endostatin | α1 chain of | 88, 277-285 (1997). |
| | type XVIII collagen | |
| Human | NC10 domanin, | Ramchandran, R et al, Biochem. |
| Endostatin- | α1 chain of | Biophys. Res. |
| like protein | type XV collagen | Comm 255, 735-739 (1999) |
| Arrestin | NC1 domain, | Kalluri et al. |
| | α1 chain of | Cold Spring Harb Symp Quant |
| | type IV collagen | Biol. 2002; 67: 255-66 |
| Canstatin | NC1 domain, | Kalluri et al. |
| Angiocol | α2 chain of | Cold Spring Harb Symp Quant |
| | type IV collagen | Biol. 2002; 67: 255-66; also |
| | | Biostratum Patent |
| Tumstatin | NC1 domain, | Kalluri et al. |
| | α3 chain of | Cold Spring Harb Symp Quant |
| | type IV collagen | Biol. 2002; 67: 255-66 |
| α6(IV) | NC1 domain, | Kalluri et al. |
| NC1 domain | α6 chain of | Cold Spring Harb Symp Quant |
| | type IV collagen | Biol. 2002; 67: 255-66. |

H. Methods of Monitoring

In one aspect, the present invention provides a method of monitoring the efficacy of one or more of the methods provided herein using any of the assays provided herein or known in the art. The subject being monitored can be a mammal such as a human or a non-human. Monitoring can determine whether the state of an angiogenesis-dependent disease or disorder has been altered by administration of any of the compositions provided herein. For example, a cancer patient can be monitored to determine if a tumor or metastasis has been eliminated, reduced in size, remained static, or grown. Alternatively, if the therapeutic method has had no effect, a determination can be made to adjust the dosage level of a composition, to begin combination therapy, to schedule surgery to remove the tumor or metastasis if not already done, or a combination thereof.

Monitoring can be, for example, any of the in vitro or in vivo diagnostic or imaging methods provided herein.

VIII. Methods of Assaying Tumor Metastasis

Tumor metastasis can be measured by a number of techniques known to those of skill in the art and published in the literature. Methods of assaying tumor metastasis using the chick embryo model (Brooks et al., Meth. Mol. Biol. 1999, 129:257-269; Testa et al., Cancer Res 1999, 59:3812-3820), and the murine model (Vantyghem, et al., Cancer Res 2003, 63:4763-4765) have been described. Subsequent histological and immunofluorescence analyses can be performed as described in the literature (Brooks, et al., Cell 1996, 85:683-693; Brooks, et al., Science 1994, 264:569-571).

IX. Methods of Assaying Angiogenesis

Methods of measuring alterations in angiogenesis are well known in the art. For example, angiogenesis can be measured in the chick chorioallantoic membrane (CAM), in a method referred to as the CAM assay. The CAM assay has been described in detail by others and has been used to measure both angiogenesis and neovascularization of tumor tissues. See Ausprunk et al., Am. J. Pathol., 1975, 79:597-618 and Ossonski et al., Cancer Res. 1980, 40:2300-2309. The CAM assay is a well-recognized assay model for in vivo angiogenesis because it involves the neovascularization of whole tissue with chick embryo blood vessels growing into either the CAM or into the tissue grown on the CAM.

The CAM assay is particularly useful because the system includes an internal control for toxicity. The health of the embryo indicates toxicity since the chick embryo itself is exposed to test reagents.

Another method for measuring alterations in angiogenesis is the in vivo rabbit eye model, referred to as the rabbit eye assay. The rabbit eye assay has been described in detail by others and has been used to measure both angiogenesis and neovascularization in the presence of angiogenic inhibitors such as thalidomide. See D'Amato et al., Proc. Natl. Acad. Sci. 1994, 91:4082-4085.

The rabbit eye assay is a well recognized assay model for in vivo angiogenesis because the neovascularization process, exemplified by rabbit blood vessels growing from the outer rim of the cornea into the cornea, is easily visualized through the naturally transparent corneal membrane. Additionally, both the extent and the amount of stimulation/regression of neovascularization can easily be monitored over time. Finally, this method has an additional benefit of indicating toxicity of the test reagent. Since the rabbit is exposed to test reagents, the health of the rabbit is an indication of toxicity of the test reagent.

Another assay, referred to as the chimeric mouse assay, measures angiogenesis in the chimeric mouse:human mouse model. This assay is described herein, and in detail by others, as a method for measuring angiogenesis, neovascularization, and regression of tumor tissues. See Yan, et al., J. Clin. Invest. 1993, 91:986-996.

The chimeric mouse assay is a useful in vivo model for angiogenesis because the transplanted skin grafts closely resemble normal human skin histologically. Additionally, neovascularization of whole tissue is occurring wherein human blood vessels are growing from grafted human skin into human tumor tissue on the surface of the grafted human skin. The origin of the neovascularization into the human graft can be demonstrated by immunohistochemical staining of the neovasculature with human-specific endothelial cell markers.

The chimeric mouse assay demonstrates regression of neovascularization based on both the amount and extent of new vessel growth. Furthermore, it is easy to monitor effects on the growth of any tissue transplanted upon the grafted skin, such as a tumor tissue. Finally, the assay is useful because there is an internal control for toxicity in the assay system. The health of the mouse is an indication of toxicity when exposed to a test reagent.

To confirm the effects of a compound, e.g., IGFBP-4, on angiogenesis, the mouse Matrigel plug angiogenesis assay can be used. Various growth factors (IGF-1, bFGF or VEGF) (250 ng) and Heparin (0.0025 units per/mL) are mixed with growth factor reduced Matrigel as previously described (Montesano, et al., J. Cell Biol. 1983, 97:1648-1652; Stefansson, et al., J. Biol. Chem. 2000, 276:8135-8141). IGFBP-4 or control BSA (10 to 500 ng) can be included in the Matrigel preparations. In control experiments, Matrigel is prepared in the absence of growth factors. Mice are injected subcutaneously with 0.5 mL of the Matrigel preparation and allowed to incubate for one week. Following the incubation period, the mice are sacrificed and the polymerized Matrigel plugs surgically removed. Angiogenesis within the Matrigel plugs is quantified by two established methods, including immunohistochemical analysis and hemoglobin content (Furstenberger, et al., Lancet. 2002, 3:298-302; Volpert, et al., Cancer Cell 2002, 2(6):473-83; Su, et al., Cancer Res. 2003, 63:3585-3592). For immunohistochemical analysis, the Matrigel plugs are embedded in OCT, snap frozen and 4 μm sections prepared. Frozen sections are fixed in methanol/acetone (1:1). Frozen sections are stained with polyclonal antibody directed to CD31. Angiogenesis is quantified by microvascular density counts within 20 high powered (200×) microscopic fields.

Hemoglobin content can be quantified as described previously (Schnaper, et al., J. Cell Physiol. 1993, 256:235-246; Montesano, et al., J. Cell Biol. 1983, 97:1648-1652; Stefansson, et al., J. Biol. Chem. 2000, 276:8135-8141; Gigli, et al., J. Immunol. 1986, 100:1154-1164). The Matrigel implants are snap frozen on dry ice and lyophilized overnight. The dried implants are resuspended in 0.4 mL of 1.0% saponin (Calbiochem) for one hour, and disrupted by vigorous pipetting. The preparations are centrifuged at 14,000×g for 15 minutes to remove any particulates. The concentration of hemoglobin in the supernatant is then determined directly by measuring the absorbency at 405 nm and compared to a standard concentration of purified hemoglobin. This method of quantification has been used successfully and has been shown to correlate with angiogenesis (Schnaper, et al., J. Cell Physiol. 1993, 256:235-246; Montesano, et al., J. Cell Biol. 1983, 97:1648-1652; Stefansson, et al., J. Biol. Chem. 2000, 276:8135-8141; Gigli, et al., J. Immunol. 1986, 100:1154-1164).

X. Methods of Assaying Cell Adhesion

Cell adhesion can be measured by methods known to those of skill in the art. Assays have been described previously, e.g. by Brooks, et al., J. Clin. Invest 1997, 99:1390-1398. For example, cells can be allowed to adhere to substrate (i.e., an ECM component) on coated wells. Non-attached cells are removed by washing, and non-specific binding sites are blocked by incubation with BSA. The attached cells are stained with crystal violet, and cell adhesion is quantified by measuring the optical density of eluted crystal violet from attached cells at a wavelength of 600 nm.

XI. Methods of Assaying Cell Migration

Assays for cell migration have been described in the literature, e.g., by Brooks, et al., J. Clin. Invest 1997, 99:1390-1398 and methods for measuring cell migration are known to those of skill in the art. In one method for measuring cell migration described herein, membranes from transwell migration chambers are coated with substrate (here, thermally denatured collagen), the transwells washed, and non-specific binding sites blocked with BSA. Tumor cells from subconfluent cultures are harvested, washed, and resuspended in migration buffer in the presence or absence of assay antibodies. After the tumor cells are allowed to migrate to the underside of the coated transwell membranes, the cells remaining on the top-side of the membrane are removed and cells that migrate to the under-side are stained with crystal violet. Cell migration is then quantified by direct cell counts per microscopic field.

XII. Methods of Assaying Tumor Growth

Tumor growth can be assayed by methods known to those of skill in the art, e.g., as described in Xu, et al., J. Cell Biol 2001, 154:1069-1079. An assay for chick embryo tumor growth can be performed as follows: single cell suspensions of CS1 melanoma (5×10⁶ per embryo) or HT1080 fibrosarcoma (4×10⁵ per embryo) are applied in a total volume of 40 μl of RPMI to the CAMs of 10-day-old embryos (Brooks et al., 1998). Twenty four hours later, the embryos receive a single intravenous injection of an inhibitor, or control molecule (100 μg per embryo). For example, if an antibody inhibitor is used, an isotype-matched antibody can serve as a control. Tumors are grown for 7 days, then re-sected and wet weights are determined. Experiments can be performed with five to ten embryos per condition.

Another method for assaying tumor growth makes use of the SCID mouse, as follows: subconfluent human M21 melanoma cells are harvested, washed, and resuspended in sterile PBS (20×10⁶ per mL). SCID mice are injected subcutaneously with 100 μL of M21 human melanoma cell (2×10⁶) suspension. Three days after tumor cell injection, mice are either untreated or treated intraperitoneally (100 μg/mouse) with an antagonist. The mice are treated daily for 24 days. Tumor size is measured with calipers and the volume estimated using the formula V=(L×W²)/2, where V is equal to the volume, L is equal to the length, and W is equal to the width.

XIII. Methods of Assaying Cell Proliferation

Cell proliferation can be assayed by methods known to those of skill in the art. As described herein, subconfluent human endothelial cells (HUVECs) can be resuspended in proliferation buffer containing low (5.0%) serum in the presence or absence of CM (25 μL) from ECV or ECVL cells, and endothelial cells allowed to proliferate for 24 hours. Proliferation can be quantified by measuring mitochondrial dehydrogenase activity using a commercially available WST-1 assay kit (Chemicon). Also, as described herein, proliferation can be quantified by measuring ³H incorporation using standard methods.

XIV. Imaging and Diagnostic Methods

A. Imaging and Diagnosis

Provided herein is a method of imaging or diagnosing angiogenesis or an angiogenic-dependent disease or disorder by contacting any of the compositions provided herein with a sample. The composition can further include an imaging or diagnostic moiety as described elsewhere herein. The sample can be, for example, blood, serum, platelets, biopsy fluid, spinal tap fluid, meninges, and urine. In one embodiment the imaging or diagnosis method is an in vitro assay. In another method, contacting includes administration of the composition to a patient and the angiogenesis or angiogenic-dependent disease or disorder is imaged or diagnosed in vivo using any of the assays and methods described herein.

B. Immunoassays

Any known methods can be used to analyze immunoreactions between the antibodies or functional fragments thereof, peptides, peptidomimetics and a bio-sample. Bio-samples to be used in the immunoassays include, but are not limited to, serum, urine, fluid from tumor biopsies, spinal fluid taps, meninges, etc. Immunoblotting, immunoprecipitation and in situ immunostaining can be used. In addition, the methods can be used in conjunction with other techniques, such as two-dimensional electrophoresis (2-DE), ultra-sensitive mass spectrometry (MS), and other high-throughout functional screening assays (Persidis, Nature Biotechnology, 1998, 16:393-394), in the proteomics studies. The examples of such 2-DE and MS analyses include, but are not limited to, isoelectric focusing followed by mass-based separation (ISO-DALT), non-equilibrium based electrophoresis (NEPHGE), and immobilized first-dimension pH gradients (IPG-DALT) (Humphery-Smith. et al. Electrophoresis, 1997 18:1217.quadrature.1242).

One technology for analyzing the immunoreactions between antibodies or functional fragments thereof and a bio-sample is tissue immunostaining, which technology is well known in the art (Feitelson & Zern, Clinics In Laboratory Medicine, W. B. Saunders Corn., 1996). Preferably, cryosected tissue samples are used to perform the immunostaining assay because the tissue sample fixed with this method can preserve the cellular antigen structure. The data from this assay may well represent the cellular protein expression pattern in the tested tissue. Alternative, paraffin fixed tissue sample can be used for antibody immunostaining because this type of tissue fixation preserves the tissue for long time and also can be easily collected from different medical research resources. There are several techniques which can be used to improve the immunostaining sensitivity when using paraffin fixed tissue samples (Lantis et al., Surgical Endoscopy., 1998, 12(2):170-176).

Another technology for analyzing the immunoreactions between peptides and a bio-sample is tissue immunostaining, which technology is well known in the art. Preferably, cryosected tissue samples are used to perform the immunostaining assay because the tissue sample fixed with this method can preserve the cellular antigen structure. The data from this assay may well represent the cellular protein expression pattern in the tested tissue. Alternative, paraffin fixed tissue sample can be used for peptide immunostaining because this type of tissue fixation preserves the tissue for long time and also can be easily collected from different medical research resources. There are several techniques which can be used to improve the immunostaining sensitivity when using paraffin fixed tissue samples (Lantis et al., Surgical Endoscopy., 1998, 12(2):170-176).

In one aspect, the present invention provides for a method of screening a patient for a collagen-dependent disease or disorder comprising: (a) obtaining a biological sample (e.g., serum, urine, fluid from tumor biopsy, spinal fluid tap, meninges) from said patient, and (b) detecting auto-antibodies to a peptide, which peptide is a fragment of an ECM component in said biological sample wherein the presence of said auto-antibodies signifies that said patient has or is predisposed to the collagen-dependent disease or disorder.

In one aspect, an embodiment provides for a method of screening a patient for a collagen-dependent disease or disorder comprising: (a) obtaining a biological sample (e.g., serum, urine, fluid from tumor biopsy, spinal fluid tap, meninges) from said patient, and (b) detecting auto-antibodies to a peptide consisting essentially of, or consisting of, an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), and variants thereof wherein P is hydroxyproline, in said biological sample wherein the presence of said auto-antibodies signifies that said patient has, or is predisposed to, the collagen-dependent disease or disorder. In one aspect, an embodiment provides for a method of screening a patient for a collagen-dependent disease or disorder comprising: (a) obtaining a biological sample (e.g., serum, urine, fluid from tumor biopsy, spinal fluid tap, meninges) from said patient, and (b) detecting auto-antibodies to a peptide consisting essentially of, or consisting of, an amino acid sequence set forth as GPPGPP (SEQ ID NO: 81) wherein one or more proline residues is hydroxyproline in said biological sample wherein the presence of said auto-antibodies signifies that said patient has, or is predisposed to, the collagen-dependent disease or disorder.

The collagen-dependent disease or disorder can be, for example, fibrocystic diseases (e.g., fibrosis and endometriosis), collagen based skin diseases (e.g., psoriasis, scleroderma, eczema), platelet based disorders associated with collagen (e.g., plaque formation, etc.), type II collagen arthritis, inflammatory diseases (e.g., restenosis, diabetic retinopathy, rheumatoid arthritis), opthalmic uses (e.g., macular degeneration), etc.

C. Microarrays

The present application encompasses microarrays that can be used to assay samples such as biosamples.

A process for identifying physiologically distinguishable markers associated with a physiologically abnormal bio-sample, which comprises: 1) assessing a proteomics profile of said physiologically abnormal bio-sample through the process of claim 1; 2) assessing a proteomics profile of a comparable physiologically normal bio-sample through the process of claim 1; and 3) comparing the proteomics profile obtained in step 1) with the proteomics profile obtained in step 2) to identify physiologically distinguishable markers associated with a physiologically abnormal bio-sample. Methods and microarrays are described, for example, in U.S. Pat. No. 6,951,742, by Mendoza et al., “High-Throughput Microarray-Based Enzyme-Linked Immunosorbent Assay (ELISA),” BioTechniques (1999) 27:778-788, and Blackstock et al., “Proteomics: Quantitative and Physical Mapping of Cellular Proteins,” Trends in Biotechnology (1999) 17(3):121-127, each of which is incorporated in its entirety by reference.

The present invention further encompasses an array of antibodies attached on a solid surface. Preferably, the antibodies used in the array specifically bind substantially to proteins or peptides isolated from a bio-sample. As used herein, antibodies encompass antibodies and functional fragments thereof as defined elsewhere herein.

The present invention further encompasses a method for assessing proteomics profile of a sample (e.g., a biosample), which method comprises: (a) dividing a plurality of antibodies into an unlabelled portion and a labeled portion; (b) attaching the unlabelled antibodies on a solid surface to form an array of unlabelled antibodies on said solid surface; (c) contacting said array of unlabelled antibodies formed in (b) with a biosample to retain antigens contained in said biosample that specifically bind to said unlabelled antibodies; (d) detecting said retained antigens by contacting said retained antigens with said labeled antibodies, thereby proteomics profile of said biosample is assessed.

In another aspect, the present invention provides an array of antibodies attached on a solid surface.

Any antibodies, whether polyclonal, monoclonal, single chain, Fc fragment, Fab fragment, F(ab)₂ fragment, F(ab′)2 fragment, scFv fragment, or a mixture thereof, can be used to produce the antibody arrays. Preferably, the array comprises antibodies that specifically bind substantially to proteins or peptides isolated from a biosample. The peptides can be isolated from physiologically normal or physiologically abnormal cells, tissues or fluids. The antibodies used in the array can be those described elsewhere herein.

The present invention further encompasses an array of peptides or peptidomimetics attached on a solid surface. Preferably, the peptides or peptidomimetics used in the array preferentially bind to an integrin from a bio-sample.

The present invention further encompasses a method for assessing proteomics profile of a biosample, which method comprises: (a) dividing a plurality of peptides or peptidomimetics into an unlabeled portion and a labeled portion; (b) attaching the unlabelled peptides or peptidomimetics on a solid surface to form an array of unlabeled peptides or peptidomimetics on said solid surface; (c) contacting said array of unlabeled peptides or peptidomimetics formed in (b) with a biosample to retain antigens contained in said biosample that specifically bind to said unlabeled peptides or peptidomimetics; (d) detecting said retained antigens by contacting said retained antigens with said labeled peptides or peptidomimetics, thereby proteomics profile of said biosample is assessed.

In another aspect, the present invention provides an array of peptides or peptidomimetics attached on a solid surface.

As used herein, the word “array” shall be taken to mean any ordered arrangement of a plurality of specified integers, including both liner and non-linear arrangements of a plurality of antibodies or functional fragments thereof, peptides, peptidomimetics or derivatives thereof. The array can be arranged on a grid, such as in microtiter wells, on a membrane support or silicon chip, or on a grid comprising a plurality of polymeric pins.

The array can be produced on any suitable solid surface, including silicon, plastic, glass, polymer, such as cellulose, polyacrylamide, nylon, polystyrene, polyvinyl chloride or polypropylene, ceramic, photoresist or rubber surface. Preferably, the silicon surface is a silicon dioxide or a silicon nitride surface. Also preferably, the array is made in a chip format. The solid surfaces may be in the form of tubes, beads, discs, silicon chips, microplates, polyvinylidene difluoride (PVDF) membrane, nitrocellulose membrane, nylon membrane, other porous membrane, non-porous membrane, e.g., plastic, polymer, perspex, silicon, amongst others, a plurality of polymeric pins, or a plurality of microtiter wells, or any other surface suitable for immobilizing proteins and/or conducting an immunoassay.

The antibodies and peptides can be attached to the solid surface by any methods known in the art (see generally, WO 99/39210, WO 99/). For example, the antibodies or peptides can be attached directly or through linker(s) to the surface. The antibodies or peptides can be attached to the surface through non-specific, specific, covalent, non-covalent, cleavable or non-cleavable linkage(s). The cleavable linkage can be cleavable upon physical, chemical or enzymatic treatment. The arrays can be arranged in any desired shapes such as linear, circular, etc.

In one example, antibody array or peptide array can be printed on a solid surface using pins (passive pins, quill pins, and the like) or spotting with individual drops of solution (WO 99/40434). Passive pins draw up enough sample to dispense a single spot. Quill pins draw up enough liquid to dispense multiple spots. Bubble printers use a loop to capture a small volume which is dispensed by pushing a rod through the loop. Microdispensing uses a syringe mechanism to deliver multiple spots of a fixed volume. In addition, solid supports, can be arrayed using piezoelectric (ink jet) technology, which actively transfers samples to a solid support. In addition, the methods disclosed in WO 95/35505 can also be used. The method and apparatus described in WO 95/35505 can create an array of up to six hundred spots per square centimeter on a glass slide using a volume of 0.01 to 100 nL per spot. Suitable concentrations of antibody range from about 1 ng/μL to about 1 μg/μL. Further, other methods of creating arrays, including photolithographic printing (Pease, et al., PNAS 91(11):5022-5026, 1994) and in situ synthesis can be used.

Methods for covalent attachment of antibodies and peptides to a solid support are known in the art. Examples of such methods are found, for example, in Bhatia, et al., Anal. Biochem. 178(2):408413, 1989; Ahluwalia, et al., Biosens. Bioelectron. 7(3):207-214, 1992; Jonsson, et al., Biochem. J. 227(2):373-378, 1985; and Freij-Larsson, et al., Biomaterials 17(22):2199-2207, 1996, all of which are incorporated by reference herein in their entirety.

Methods of reducing non-specific binding to a solid surface are well known in the art and include washing the arrayed solid surface with bovine serum albumin (BSA), reconstituted non-fat milk, salmon sperm DNA, porcine heparin, and the like (see Ausubel, et al., Short Protocols in Molecular Biolog, 3rd ed. 1995).

A method for assessing proteomics profile of a biosample is also provided herein, which method comprises: 1) dividing a plurality of antibodies into an unlabelled portion and a labeled portion; 2) attaching the unlabelled antibodies on a solid surface to form an array of unlabelled antibodies on said solid surface; 3) contacting said array of unlabelled antibodies formed in step 2) with a biosample to retain antigens contained in said biosample that specifically bind to said unlabelled antibodies; and 4) detecting said retained antigens by contacting said retained antigens with said labeled antibodies, thereby proteomics profile of said biosample is assessed.

Preferably, the plurality of antibodies used in the above methods are produced and characterized against a denatured collagen. Exemplary antibodies include, but are not limited to antibodies that preferentially bind to a polypeptide having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), and variants thereof wherein P is hydroxyproline. Exemplary antibodies include, but are not limited to antibodies that preferentially bind to a polypeptide having an amino acid sequence set forth as GPPGPP (SEQ ID NO: 81) wherein one or more proline residues is hydroxyproline.

A method for assessing proteomics profile of a biosample is also provided herein, which method comprises: 1) dividing a plurality of peptides or peptidomimetics into an unlabeled portion and a labeled portion; 2) attaching the unlabeled peptides or peptidomimetics on a solid surface to form an array of unlabeled peptides or peptidomimetics on said solid surface; 3) contacting said array of unlabeled peptides or peptidomimetics formed in step 2) with a biosample to retain antigens contained in said biosample that specifically bind to said unlabeled peptides or peptidomimetics; and 4) detecting said retained antigens by contacting said retained antigens with said labeled peptides or peptidomimetics, thereby proteomics profile of said biosample is assessed.

Preferably, the plurality of peptides are fragments of a denatured ECM component, such as, for example, collagen or a denatured collagen. Exemplary peptides have an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36), and variants thereof wherein P is hydroxyproline. Exemplary peptides have an amino acid sequence set forth as GPPGPP (SEQ ID NO: 81) wherein one or more proline residues is hydroxyproline. In one non-limiting embodiment, the peptide or peptidomimetic consists essentially of, or consists of, an amino acid sequence as described herein.

A method for identifying physiologically distinguishable markers associated with a physiologically abnormal bio-sample is further provided herein, which method comprises: 1) assessing proteomics profile of said physiologically abnormal bio-sample through the above-described method; 2) assessing proteomics profile of a comparable physiologically normal bio-sample through the above-described method; and 3) comparing the proteomics profile obtained in step 1) with the proteomics profile obtained in step 2) to identify physiologically distinguishable markers associated with a physiologically abnormal bio-sample. Preferably, the physiologically abnormal bio-sample is isolated from an organism, preferably mammals or humans with a disease or disorder, and the method is used in prognosis, diagnosis, or monitoring treatment of such diseases or disorders. The exemplary the diseases or disorders that can be monitored by the present methods include angiogenesis-dependent disorders, collagen-dependent disorders, and cell proliferative disorders as described supra.

The above described processes, methods and antibody arrays can be used for identifying physiologically distinguishable markers associated with a physiologically abnormal bio-sample, or for identifying substances that modulate proteomics profile of a biosample.

D. Methods of Selecting Cells

Flow cytometry is the first single cell analysis method with the potential to identify and isolate, enrich or purify, the distribution of cellular properties within a large number of cells. In flow cytometry, cells travel in a liquid stream, and each single cell, as it passes the exciting light and the measuring optics, sends out a number of signals, including the size and structure related forward and side scatter and the fluorescent signals, which in turn are dependent on the staining procedure that has been used. These signals are measured and stored for each individual cell. Flow cytometry allows for rapid analysis, identification and enrichment/purification of millions of cells and is useful for identifying and isolating rare populations of cells based on a cell surface moiety.

Cell sorting can be used for any technique that separates cells according to their properties. Such techniques include panning, fluorescence activated cell sorting (FACS) or magnetic cell sorting (MACS). Panning, FACS and magnetic cell sorting can be used for the selection of cells according to the expression of a surface molecule. Such methods are known in the art and can be found as described by Mattanovich and Borth. Microbial Cell Factories 2006, 5:12.

In one non-limiting embodiment, a sample can be contacted with a labeled antagonist of the present invention. Labels are well known in the art and can include imaging and/or therapeutic labels such as, but not limited to, fluorescent labels (e.g., PE, FITC, Texas Red, cytochrome C, propidium iodide (PI), etc) for visualization. Samples from patients to be diagnosed can be labeled with such labeled antagonists and analyzed using any of the visualization assays described herein, such as FACS or flow cytometry in combination with cell sorting to enrich cell populations. The presence of a ligand to an antagonist of the present invention above the level of negative controls can indicate the likelihood of a collagen-dependent disease or disorder, neovascularization, a cell-proliferative disorder or an angiogenesis-dependent disorder.

In the event that a patient is diagnosed as having a positive level of ligand in the sample, the diagnosis can further be confirmed using any accepted means in the art. Following diagnosis, the patient can be administered any of the antagonists provided herein. Optionally, the antagonist can be labeled with a therapeutic moiety to selectively kill cells at a site of neovascularization or angiogenesis.

XV. Methods of Stimulating Growth

Molecular alterations that occur in both tumor and stromal cells are thought to potentiate angiogenesis in part by modifying expression and bioavailability of angiogenic growth factors as well as altering expression of matrix-degrading proteases. Collectively, these and other molecular changes help to create a microenvironment conducive to new blood vessel growth, one factor that contributes to cell and tissue growth. There is evidence for the importance of numerous molecular regulators that contribute to new blood vessel growth, including matrix-degrading proteases such as MMP-9, angiogenesis inhibitors such as TSP-1 and angiogenic growth factors such as VEGF (see, e.g., Yu, et al., Proc. Natl. Acad. Sci. USA 1999, 96:14517-14522 and Dameron, et al., Science 1994, 265:1582-1584). These molecular regulators, the proteins that in turn regulate them, and any of a number of other molecules potentially affect angiogenesis and metastasis.

Studies have suggested that angiogenesis requires proteolytic remodeling of the extracellular matrix (ECM) surrounding blood vessels in order to provide a microenvironment conducive to new blood vessel development (Varner, et al., Cell Adh. Commun. 1995, 3:367-374; Blood, et. al., Biochim. Biophys. Acta. 1990, 1032:89-118; Weidner, et al., J. Natl. Cancer Inst. 1992, 84:1875-1887; Weidner, N. et al., N. Engl. J Med. 1991; 324:1-7; Brooks, P. C. et al. J Clin. Invest. 1995; 96:1815-1822; Brooks, P. C. et al., Cell 1994; 79:1157-1164). The extracellular matrix protein, collagen, makes up over 25% of the total protein mass in animals and the majority of protein within the ECM. Proteolytic exposure of unique matrix immobilized cryptic epitopes and subsequent cellular interactions with these epitopes, which serve as regulatory sites, play crucial roles in angiogenesis, tumor growth and metastasis.

Extracellular matrix components include, e.g., collagen, fibronectin, osteopontin, laminin, fibrinogen, elastin, thrombospondin, tenascin, and vitronectin. Studies have identified cryptic sites, including those are preferentially bound by the antibody HUI77 and fragments and fragments thereof, that regulate proliferation of melanoma cells, and by extension, angiogenesis, neovascularization and angiogenic-dependent disorders (U.S. application Ser. No. 10/011,250 the subject matter of which is incorporated by reference herein in its entirety). Methods of generating synthetic or bioartificial tissues, such as skin, are described, for example, in U.S. Pat. Nos. 7,169,382 and 6,960,427, each of which is incorporated by reference herein.

Thus, one embodiment of the present invention relates to generating in vitro preparations of synthetic skin that can be used in wound healing and to treat burn victims. Such preparations and methods include coating one or more plates with collagen to promote growth of skin and adding peptides with other agents and factors such as, for example, keratinocytes. Exemplary peptides include, but are not limited to polypeptides having an amino acid sequence set forth as PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) or FPGPPGPDGLPGSMGPPG (SEQ ID NO: 36) or a variant thereof, wherein P is hydroxyproline. Exemplary peptides include, but are not limited to polypeptides having an amino acid sequence set forth as GPPGPP (SEQ ID NO: 81) wherein one or more proline residues is hydroxyproline. In one non-limiting embodiment, the peptide or peptidomimetic consists essentially of, or consists of, an amino acid sequence as described herein. Such peptides can be added to the plates in combination with other known growth factors to generate synthetic skin.

### EXAMPLES

The present invention is further illustrated by the following examples, which should not be construed as limiting in any way.

### Reagents and Chemicals

Human collagen type IV, proteomics grade trypsin, bovine serum albumin (BSA), streptavidin, phosphate buffer saline, adenosine, cytosine, thymidine, guanosine, Tween-20, 2N sulfuric acid and dimethyl sulfoxide were all obtained from Sigma-Aldrich (St. Louis, Mo.). Methanol was purchased from EMD Chemicals (Gibbstown, N.J.). Pre-cast polyacrylamide gels, sample loading buffer and 10× electrophoresis buffer were purchased from Biorad (Hercules, Calif.). Secondary anti-human IgG conjugated to horseradish peroxidase was purchased from Jackson ImmunoResearch (West Grove, Pa.). Tetramethylbenzidine (TMB) substrate was purchased from Pierce Chemicals (Rockford, Ill.).

### Methods

1. Elisa to Measure Binding of Anti-Denatured-Collagen Antibodies

Binding of D93 to human dn-collagen IV was evaluated using a D93 ELISA for measuring binding to denatured-collagen).

Human dn-collagen IV was prepared using standardized procedures. Briefly, the protocol entailed incubation of a 10 μg/mL solution of human collagen IV at 56° C. for 30 minutes and dispensing 12 mL aliquots to 300 15 mL conical plastic tubes. The tubes were stored at −80° C. Random tubes were selected and thawed for use.

The ELISA was conducted as follows: wells of a 96-well microtiter plate (Immulon, 4 HBX flat-bottomed, Thermo Electron Corporation, Waltham, Mass.) were coated with 100 μL/well of dn-collagen IV at 10 μg/mL in Phosphate Buffered Saline (PBS) overnight at 4° C. The plate was washed 3 times (3×) with PBS containing 0.05% (v/v) Tween-20 (PBST) using an automated plate washer.

The plate was blocked by adding 300 μL/well of 1% BSA (w/v) in PBS for 1 hour at 25° C. followed by washing 3× in PBST as above.

Solutions of D93 or HUI77 were prepared with various concentrations of peptide or antibody inhibitor and incubate for 15 minutes. D93 or HUI77 was prepared at 200 ng/mL in 1% BSA/PBS.

For antibody blocking studies using synthetic peptides: peptide inhibitor stock solutions were prepared at 2 mM, 0.2 mM, 0.02 mM, and 0.002 mM in 1% BSA/PBS. 500 μL of a 2 mM peptide solution in 1% BSA/PBS was prepared and diluted 1:10 in 500 μL a total of three times to make peptide inhibitor concentrations.

For D93 antibody cross-blocking of HUI77: D93 inhibitor solutions were prepared using 100, 20, and 10 μg/mL in 1% BSA/PBS.

200 μL of D93 or HUI77 at 200 ng/mL solution was combined with 200 μL of peptide or antibody inhibitor solution and incubated for 15 minutes at room temperature.

D93 or HUI77 with various concentrations of inhibitors (2 mM, 0.2 mM, 0.02 mM, and 0.002 mM) were added to microplate wells and incubated for 1 hour at 25° C. The plate was washed 3× in PBST as above.

To each microtiter plate well, 100 μL of diluted anti-human IgG or anti-mouse IgM conjugated to horseradish peroxidase (HRP) in PBST was added and incubated for one hour at 25° C.: (a) for D93: Goat anti-human IgG-HRP (Jackson ImmunoResearch, Catalog No. 109-035-088) was used at 1:20,000 and (b) for HUI77: Goat anti-mouse IgM-HRP was used at 1:4,000. Plates were washed 3× in PBST as above.

Tetramethylbenzidine (TMB) substrate (100 μL; Pierce Chemicals)) was added to wells and the plates were allowed to develop at room temperature for 15 minutes in the dark. Substrate development was stopped by adding 50 μL/well of 2N H2SO4. The microtiter an end-over-end mixer. A plates were read at 450 nm within five minutes of adding the H2SO4. Reactivity was measured with a microplate reader (Molecular Devices, Sunnyvale, Calif.) at a wavelength of 450 nm and analyzed using SOFTMax® PRO4.0 (Molecular Devices). All samples were tested in triplicate.

2. Preparation of Human Collagen Fragments by Proteolytic Digestion with Trypsin or Thermal Denaturation

Human collagen IV was dissolved in 50 mM acetic acid at 5 mg/mL overnight at 4° C. in a 15 mL plastic tube using buffer exchange of the dissolved human collagen IV/acetic acid solution was performed using a 10K molecular weight cut-off dialysis cassette and dialyzing against 2×1 L volumes of 10 mM Tris pH 7.5 for a total of 18 to 24 hours at 2 to 8° C. The dialyzed collagen IV solution was stored at −20° C.

Alternatively, human collagen IV was dissolved in 50 mM acetic acid at 1 mg/mL for 1-2 hours at room temperature using an end-over-end mixer. The dissolved collagen IV solution was diluted 1:100 in 1 L of PBS pre-warmed to 56° C. in a 1 liter plastic vesicle to achieve a final 10 μg/mL concentration. The collagen IV/PBS solution was incubated for 30 minutes at 56° C., aliquoted in conical tubes and stored at −80° C.

In a pilot study, five different ratios (w/w) of collagen and proteomics-grade trypsin were used to titrate the optimum conditions for collagen fragment preparation. One vial of proteomics grade trypsin (20 μg) was dissolved in dH20 (20 μL) to achieve a final concentration of 1 mg/mL trypsin solution. Dialyzed collagen IV solution (50 μL) was pipetted into six 1.7 mL microfuge tubes using a P-200 pipettor. Trypsin was serial diluted into the individual microfuge tubes containing the dialyzed collagen solution by transfer of 5 μL trypsin stock into Tube No. 1 and followed by serial transfer of 5 μL the collagen/trypsin mixture into the subsequent microfuge tubes as shown in Table 9. Trypsin digestion of collagen was conducted at 37° C. for 18 hours in using a heat block.

TABLE 9
Titration of Trypsin for Preparation
of Collagen Peptide Fragments

| | COLLAGEN/TRYPSIN TITRATION TUBE NO. | | | | | |
|---|---|---|---|---|---|---|
| | 1 | 2 | 3 | 4 | 5 | 6 |
| Collagen | 50 μL | 50 μL | 50 μL | 50 μL | 50 μL | 50 μL |
| Solution | | | | | | |
| (5 mg/mL) | | | | | | |
| Trypsin | 5 μL of | 5 μL | 5 μL | 5 μL | 5 μL | none |
| Solution | trypsin | of Tube | of Tube | of Tube | of Tube | |
| (1 mg/mL) | solution | No. 1 | No. 2 | No. 3 | No. 4 | |

Based on SDS/PAGE and Western blot analysis (see below) of the titration of trypsin digestion of collagen, the optimum condition for scale up of 1 mL (˜5 mgs) of trypsin-digested collagen fragments was selected from conditions in Tube No. 1 (see Table 1). For the larger scale preparation of trypsin-digested fragments, 1 mL of dialyzed collagen stock solution (5 mg/nL) and 50 μL of trypsin solution (1 mg/mL) were mixed in a 1.5 mL microfuge tube and incubated at 37° C. for 18 hours in a heat block. Following the incubation period, 5 μL was subjected to SDS/PAGE for confirmation of collagen, and 5 μL was tested for reactivity to D93 by Western blot analysis.

3. SDS-PAGE

SDS-PAGE electrophoresis of collagen peptides was performed according to conventional procedures for size separation of proteins. In separate 1.5 mL microfuge tubes, 20 μL of the collagen-trypsin mixture described in Table 1 was combined with 40 μL 2× Tris/tricine sample buffer with 5% (v/v) 2-mercaptoethanol. The microfuge tubes were heated for ten minutes in a 100° C. heat block and then briefly centrifuged for ten seconds at 12,000 rpm in a microfuge to place the contents at the bottom of the tube. A 10-16.5% PAGE pre-cast gel was assembled in the electrophoresis tank, and the tank was filled with 1×SDS/PAGE Tris/Tricine electrophoresis buffer (˜750 mL). The individual wells of the pre-cast gel were flushed with electrophoresis buffer using a P-200 pipettor/with tip to remove potential gel fragments from the well prior to loading the samples. To each well 15 μL of trypsin-digested collagen IV peptides sample (˜5 μg collagen/trypsin) or blank 1× sample buffer was loaded. Pre-stained protein molecular weight markers were loaded in lanes adjacent to the samples. Electrophoresis was conducted for 1 to 1.5 hours at a constant 100V. Following completion of the electrophoresis step, the pre-cast gel was disassembled and soaked in 2×500 mL volumes of deionized water in a glass dish for ten minutes each on a rotating platform at 50 to 60 rpm to remove the SDS. The protein bands were visualized by immersing the gel in 200 mL of Coomassie Blue (BioSafe, BioRad) for 30 to 60 minutes at room temperature on a shaking platform at 50 to 60 rpm. The gel was de-stained with two changes of 500 mL volumes of deionized water for ten minutes for each incubation. A picture of the gel was captured using a digital camera (VersaDoc™ system), and the image was stored as a .tif file on a computer.

4. Electrophoretic Transfer of Proteins to PVDF Membrane and Western Blot Analysis

Samples of trypsin-digested collagen IV were subjected to SDS/PAGE using methods stated in above. Following electrophoresis, the gel was disassembled and placed into a 10″×10″ glass dish containing electrophoresis blot transfer buffer. A standard electrophoresis transfer blot “sandwich” was prepared to transfer proteins from an acrylamide gel to a PVDF membrane using the Protein 3 mini-gel/transfer blot system (Egger, D, and Bienz, K (1994) Protein (western) blotting. Mol. Biotechnol. 1, 289-305). A piece of PVDF membrane (Immobilon-P, Millipore) was cut using scissors to the same dimensions as the acrylamide gel and pre-wetted in a glass dish containing 100 mL of methanol for five minutes. Following the pre-wetting step, the PVDF membrane was transferred to a glass dish containing deionized water for five minutes, then to a separate glass dish containing 100 mL electrophoresis blot transfer buffer. The acrylamide gel was placed oriented on top of the PVDF membrane and sandwiched between two pieces of filter paper that were placed between two pre-cut sponges and fitted into an electrophoresis blot cassette. The blot cassette was placed into an electrophoresis tank containing ˜750 mL electrophoresis blot buffer, and transfer was conducted at 100V constant for one hour at room temperature. Following transfer, the transfer cassette was disassembled, and the PVDF membrane was rinsed in 500 mL deionized water in a clean glass dish. The PVDF membrane was either air-dried or processed immediately for Western blot analysis or protein sequencing.

For total protein staining, a section of the membrane was stained with 50 mL of Coomassie Blue Staining Solution in a small plastic container for one minute, followed by destaining for two to five minutes with 40% methanol (v/v) in deionized water. For Western blots, membranes were incubated with 50 mL of 5% non-fat milk in PBS/0.1% sodium azide (BLOTTO) in a clean plastic container for 12 to 18 hours at 2 to 8° C. on a rocker platform at low-speed (just fast enough to keep the membrane floating). The membrane was transferred to a fresh plastic container and washed three times with 100 mL PBST for five minutes each on a platform shaker at 50 to 60 rpm at room temperature. Blots were probed with antibody at 10 μg/mL in 50 mL BLOTTO in a small plastic container on a shaking platform for one hour at room temperature. Membranes were washed 3× with PBST and incubated with a goat anti-human IgG-peroxidase (1:2,000) in PBST for one hour at room temperature on a platform shaker. Membranes were washed 3× with PBST and incubated with DAB peroxidase substrate solution for ten minutes until protein bands appeared. A picture of the developed membrane was captured using a digital camera (VersaDoc™ system), and the image was stored as a .tif file on a computer.

5. N-Terminal Sequencing of Collagen Peptides by Edman Degradation and Protein Sequence Alignments

PVDF membranes containing collagen peptides were stained with Coomassie Blue Staining Solution (BioRad) R-250 (0.5% in 50% methanol) for one minute in a clean plastic dish to visualize the proteins. The PVDF membrane was removed to clean glass dish containing 100 mL Coomassie Blue Destaining Solution and allowed to destain until the protein bands were barely visible (membrane was still dark blue). The membrane was air-dried on filter paper (protein bands became more visible and background less blue). Protein sequencing was performed by Edman degradation (Deutzmann, R. (2004) Structural characterization of proteins and peptides. Methods Mol. Med. 94, 269-297; Edman, P. (1970) Mol. Biol. Biochem. Biophys. 8, 211-255) directly from the PVDF membranes at the Department of Biological Sciences, University of California, San Diego, La Jolla, Calif., using a Procise Sequencer (Applied Biosystems, Foster City, Calif.). Approximately 10 to 15 amino acid residues were obtained from each peptide per sequencing reaction.

Sequences determined from results of protein sequencing reactions were compared to the GenBank® database at the world wide website “ncbi.nlm.nih.gov/entrez” using the BLAST comparison program. Alignment of collagen chains from different species was performed using the Clustal W program from the world wide website “ebi.ac.uk.” Referenced post-translation modifications such as the location of hydroxyproline of collagens was obtained from the Integrated Protein Classification Database (iProClass) located on the Protein Information Resource (PIR) database website at “pir.georgetown.edu”.

6. Screening of Synthetic Peptides Reacting to D93 and HUI77

The synthesis of short peptide arrays or single peptides was contracted to New England Peptide, Gardner, Mass. A peptide array (primary array) was initially constructed which corresponded to specific locations in the protein sequences that were identified in by N-terminal sequencing reactions as described above. The peptide array consisted of a 96-well microplate template containing individual, overlapping peptides (˜1 to 10 mg/well) corresponding to a contiguous sequence of the human collagen type alpha I chain (between residues G1,064 and T1,669) with an N-terminal biotin and 50% hydroxyproline or proline mixture for each proline residue, with a length of 16 amino acids and overlap of 10 amino acids with the adjacent protein sequence. Peptides were dissolved in 400 μL DMSO to achieve a stock peptide concentration of 2.5 to 25 mg/mL.

The ELISA assay described above was used screen peptides for D93 binding activity. Briefly, 100 μL of streptavidin at 10 μg/mL in PBS was coated onto 96-well microtiter plates from 12 to 18 hours at 2 to 8° C. Plates were blocked with 1% BSA/PBS as above. D93 was diluted to 10 μg/mL in 1% BSA/PBS, and 100 μL per well was added, in duplicate, as described above and incubated for 1 hour at room temperature. The secondary anti-human IgG-HRP, substrate development and plate reader steps were as described above. Peptides which were bound by D93 were further tested for inhibition of D93 binding to human heat-denatured-collagen IV using the ELISA described above. Peptides and D93 antibody were prepared at a 2× concentration in 1% BSA/PBS of the final test concentration and pre-mixed in 1.7 mL microfuge for 15 minutes prior to adding to the microplate wells.

A second peptide array was constructed based on permutations of the protein sequence (collagen IV alpha I chain residues 1337-1352, termed “peptide 40” of the primary array) in the primary array that reacted with D93. The secondary array consisted of individual peptides with either proline or hydroxyproline at each proline residue in the peptide PGAKGLPGPPGPPGPY (SEQ ID NO: 1).

Individual peptides with amino acid substitutions and deletions were also synthesized based on the primary and secondary peptide arrays. These peptides were dissolved in deionized water to a concentration of 10 mM since DMSO was observed to inhibit both D93 and HUI77 binding to dn-collagen IV. The inhibitory activity of the individual peptides was tested by ELISA as previously described to measure binding of D93 or HUI77 to denatured collagen where peptides and antibodies were prepared at a 2× concentration and pre-incubated for 15 minutes prior to adding to microtiter plates coated with dn-collagen.

7. Competition Assay for Screening Synthetic Peptides that Block Binding of D93 and HUI77 to Human Heat Denatured Collagen IV

96-well plates were coated with 100 μL/well of denatured (dn) collagen IV at 10 μg/mL in phosphate buffered saline (PBS) overnight at 4° C. followed by washing the plates in an automated plate washer 3 times in PBS containing 0.05% (v/v) Tween-20 (PBST). Non-specific binding was prevented by blocking the wells by adding 300 μL/well of 1% bovine serum albumin (BSA) (w/v) in PBS for 1 hour (hr) at 25° C. The plates were washed as above.

Solutions of D93 and HUI77 antibodies were prepared with various concentrations of inhibitor and incubated for 15 minutes. Briefly, 20 mLs of D93 or HUI77 were prepared at 200 ng/mL in 1% BSA/PBS. Solutions of peptide inhibitors (2 mM, 0.2 mM, and 0.002 mM) were prepared in 1% BSA/PBS. Equal volumes of antibodies and peptide inhibitors were combined: 200 μL of D93 or HUI77 (200 ng/mL solution) with 200 μL of each peptide solution.

100 μL/well of D93 or HUI77 at 100 ng/mL with various concentrations of inhibitors were added to the wells in triplicate and incubated for 1 hour at 25° C. The plates were washed as described above.

100 μL/well of secondary horseradish peroxidase (HRP)-conjugated antibody in PBST was added to each well and incubated for 1 hour (hr) at 25° C. For D93, goat anti-human IgG-HRP (Jackson ImmunoResearch) was used at 1:20,000; for HUI77, goat anti-mouse IgM-HRP (Jackson ImmunoResearch) was used at 1:4,000. The plates were washed as above.

100 μL/well of TMB substrate (Pierce) was added to each well for 15 minutes and allowed to develop in the dark. 50 μL/well of 2N sulfuric acid (H₂SO₄) was added to each well to stop the reaction and the plate was read at 450 nm within 4 minutes of adding sulfuric acid.

8. Kinetic Measurement of D93 Antibody Binding to Denatured Collagen by Surface Plasmon Resonance

Denatured-collagen was immobilized onto a research grade CM5 sensor chip using standard amine coupling. Each of three surfaces was first activated for seven minutes using a 1:1 mixture of 0.1 mM N-hydroxysuccinimide (NHS) and 0.4 mM 1-ethyl-3-(3-dimethylaminopropyl)-carbodimide (EDC). Then, the dn-collagen sample was diluted 1- to 50-fold in 10 mM sodium acetate, pH 4.0, and exposed to the activated chip surface for different lengths of time (ten seconds to two minutes) to create three different density surfaces of collagen. Each surface was then blocked with a seven-minute injection of 1 M ethanolamine pH 8.2. Biotinylated peptide was diluted 100-fold and injected for different amounts of time to be captured at three different surface densities (60 RU, 45 RU, 12 RU; Response Unit (RU) is termed by Biacore and relates to target molecule per surface area) onto a streptavidin-containing sensor chip. All experiments were performed on a Biacore® 2000 or T100 optical biosensor. D93 or DP28 antibody supplied at 100 μg/mL (or 1.32 μM based on a molar concentration of 2 Fab regions and a predicted molecular weight for D93 of ˜150 kDa) and tested in a 3-fold dilution series in Sample Running Buffer over the three collagen or peptide surfaces. Each of the D93 concentrations (five 3-fold dilutions of dn-collagen-coupled sensors, six 3-fold dilutions for peptide-coupled sensor) was tested three times to assess reproducibility of the assay. Each test was injected at a flow rate of 100 μL/minute for 60 seconds, followed by a three-minute dissociation phase. Bound D93 or DP28 was removed using a five-second pulse with sensor regeneration solution. All data were collected at a temperature-controlled 20° C. The kinetic responses for the MAb injections were analyzed using the non-linear least squares analysis program CLAMP (Myszka, D. G. and Morton, T. A. (1998) Trends Biochem. Sci., 23: 149-150). Calculations of multivalent interactions were determined using a model to fit the avidity of the bivalent interaction of D93 with dn-collagen or the synthetic peptides (Drake et al. (2004) Anal. Biochem., 328: 35-43; and Muller et al., (1998) Anal. Biochem., 261: 149-158).

### Example 1

### Monoclonal Antibody HUI77

This example describes the generation of a denatured collagen specific monoclonal antibody, Mab HUI77.

Mab HUI77 was generated and isolated by the immunological technique termed subtractive immunization (S.I). The subtractive immunization technique allows one to experimentally manipulate the immune response within mice to selectively enhance an immune response to a rare and/or low abundant epitope within a mixture of common highly antigenic epitopes. Briefly, female BALB/c mice were injected intraperitoneally with either native human triple helical collagen type-I or type-IV. At 24 and 48 hours following the injections of triple helical collagen, the mice were injected with the tolerizing agent cyclophosphamide to kill activated B-cells that would produce antibodies directed to common immunodominant epitopes within native triple helical collagen type-I and type-IV. Following the tolerization protocol, the mice were next injected with thermally denatured human collagen type-I or type-IV to stimulate an immune response to epitopes exposed following thermal denaturation. Collagen was denatured by boiling for 15 minutes. The injections of thermally denatured collagen type-I and type-IV were given every three weeks for a total of 4 to 5 injections. Serum from each mouse was tested for immunoreactivity with both native triple helical and denatured collagens. The mice demonstrating the highest titer for reactivity to denatured collagen as compared to triple helical collagen were used for the production of hybridomas. Spleen cells from the selected mice were fused with myeloma cells by standard techniques. Individual hybridoma clones were tested for the production of antibody to either triple helical or denatured collagen type-I and type-IV. Hybridoma clones were selected that produced antibodies that demonstrated a selective reactivity to denatured collagen type-I or type-IV as compared to native triple helical collagens type-I and type-IV. Mabs were purified by standard techniques.

HUI77 was shown to specifically recognize denatured collagens type-I and type-IV but binds to native triple helical collagens type-I and type-IV with substantially reduced affinity (data now shown). In particular, HUI77 binds to denatured collagen type-I with an apparent reactivity of at least about 10-fold higher than that of native collagen type-I as measured by ELISA. HUI77 also binds to denatured collagen type-IV with an affinity of about 10-fold higher than for native collagen type-IV. In addition, HUI77 does not bind substantially to other matrix components such as laminin, fibronectin, vitronectin or fibrinogen, thus demonstrating its specificity to a cryptic epitope within collagens type-I and type-IV.

HUI77 also is specific for other denatured collagens and binds the native forms of these collagens with substantially reduced affinity. HUI77 also binds denatured collagens III, IV and V with about 7-fold, about 8-fold, and about 10-fold more tightly than the respective native forms of these collagens using ELISA (data not shown).

The amino acid sequence of HUI77 is provided as SEQ ID NO: 94.

### Example 2

### Generation of CDR Variant Libraries of the HUI77 Antibody

This example describes the generation of CDR variant libraries of the HUI77 antibody for CDR optimization.

The CDR3 regions of antibodies HUI77 were optimized by generating a library of CDR variants. Primers for light chain CDR3 and heavy chain CDR3 were used to generate a library of CDR3 variants, where the primer was synthesized to encode more than one amino acid one or more positions in CDR3. Following synthesis of primers encoding CDR3 variants, the variant CDR3 regions were assembled into light chain (VL) and heavy chain (V_{H}) regions.

Briefly, humanized V_{L} and V_{H} genes of HUI77 antibody were assembled with primers using PCR or primer-elongation-ligation as described in U.S. Ser. No. 10/011,529, which is incorporated herein in its entirety by reference. Variable region genes containing CDR3 mutations were assembled by replacing the wild type CDR3 primer (IV26-17, IV26-h7, I77-17 or I77-h7) with the group of mutant primers corresponding to that CDR. The assembled variable regions were then amplified and asymmetrically biotinylated on plus strand by PCR using primers B-pelB and 224 for V_{L} and B-phA and 1200a for V_{H} genes.

The assembled V_{L} and V_{H} regions were introduced into a Fab expression vector by mutagenesis. Briefly, the non-biotinylated minus strands were isolated after binding the PCR products to NeutrAvidin-conjugated magnetic beads and introduced into the Fab expression vector IX-104CSA by hybridization mutagenesis (Kristensson et al., Vaccines 95, pp. 39-43, Cold Spring Harbor Laboratory, Cold Spring Harbor (1995); Kunkel, Proc. Natl. Acad. Sci. USA 82:488-492 (1985); Wu et al., J. Mol. Bio. 294:151-162 (1999)).

Three humanization-CDR3-mutation libraries were constructed for each the HUI77 antibody. The three libraries introduced random mutations but differed in CDR3 mutations. One library had mutations only in LCDR3, the second library had mutations only in HCDR3, and the third library had mutations in both LCDR3 and HCDR3.

Methods essentially the same as those described above for CDR3 mutagenesis were also performed on CDR1 and CDR2 of the HUI77 antibody. After assembling into a Fab expression vector, the Fabs containing HUI77 variant CDRs were expressed in bacteria and tested for binding to denatured collagen. The mutant libraries were screened with filter lift screening and ELISA. The assays were performed essentially as described previously (Huse et al., J. Immunol. 149:3914-3920 (1992); Watkins et al., Anal. Biochem. 253:37-45 (1997)). Briefly, nitrocellulose membranes were pre-coated with heat-denatured human collagen I or IV and used to lift E. coli-expressed variant Fabs from phage plates. The membranes were then incubated with antibodies, either anti-human kappa chain or anti-hemaglutinin (HA) tag conjugated to alkaline phosphatase, to detect bound variant Fabs. Positive clones were screened again by single point ELISA (Watkins et al., supra, 1997) for binding to denatured-biotinylated human collagen I and IV, correspondingly. Beneficial variants were characterized for binding to both collagens in native and heat-denatured forms by ELISA. Beneficial mutations were determined as those having higher affinity binding to denatured collagen relative to the corresponding wild type Fab, as demonstrated by ELISA.

D93 represents one variant of HUI77 and has a heavy chain CDR1 referenced as SEQ ID NO: 94; a heavy chain CDR2 referenced as SEQ ID NO: 95; a heavy chain CDR3 referenced as SEQ ID NO: 96; a light chain CDR1 referenced as SEQ ID NO: 97; a light chain CDR2 referenced as SEQ ID NO: 98; and a light chain CDR3 referenced as SEQ ID NO: 99.

Other variants of HUI77 are as described in U.S. Ser. No. 10/011,250, which is incorporated herein in its entirety by reference.

### Example 3

### Identification of Binding Sites on Denatured Collagen

Several binding sites for D93, a recombinant humanized IgG₁ kappa antibody targeting denatured-collagen have been identified on collagen type IV. Proteolytic fragments of collagen IV were identified by Western blot analysis and subjected to protein sequencing by Edman degradation. Three peptides with an approximate size of 23, 35, and 57 kDa were shown to have N-terminal sequences consistent within the α1 chain of collagen type IV. Using D93 as a probe, a peptide with the sequence Hyp-G-A-K-G-L-P-G-P-Hyp-G-P-Hyp-G-P-Y (SEQ ID NO: 2) was identified by direct binding of a synthetic peptide array of the C-terminal region of the triple-helical region of collagen type IV (Hyp=hydroxyproline). Amino acids found to be important for maximum inhibition of D93 binding to denatured-collagen were identified as G-P-Hyp-G-P-Hyp-G-P-Y (SEQ ID NO: 30), with a strong dependence on the presence of hydroxyproline. The same peptide sequence was shown to inhibit HUI77 binding to denatured-collagen type IV. A database search of homologous peptide sequences to the peptide array sequence that bound D93 and HUI77 indicates that both the alpha 1 and alpha 2 chains of collagen type IV have multiple potential binding sites. Potential D93 and HUI77 binding sites with identical and homologous peptide sequences to those identified in collagen type IV are also observed in other collagen types. In addition, the same identical and homologous sequences were observed on collagen types from other species, including chicken and mouse.

These data support the observation that D93 blocks the binding of HUI77 to human denatured-collagen IV. The presence of these peptide sequences in collagens of several species support the observation that D93 and HUI77 bind to denatured-collagens from several species as shown by ELISA. Binding inhibition of both D93 and HUI77 by monomeric G-X-Hyp-G motif suggests that many permutations of this sequence may be involved in binding of these antibodies to denatured-collagens. The location of these sequences may be important in determining the mechanism(s) by which D93 inhibits potential cellular and protein interactions within the interstitial and basement membrane ECM of tumors.

D93 and the murine IgM anti-denatured-collagen antibody HUI77 were shown to bind specific peptide sequences within the triple-helical region of collagen type IV. The sequence of peptides identified by Western blot analysis using D93 was identified in a synthetic peptide array of the collagen type IV alpha I chain. The sequences associated with D93 and HUI77 binding were also shown to be present in both the alpha 1 and alpha 2 chains of collagen types I and IV of multiple species. These binding data support the observed anti-angiogenic effects of both D93 and HUI77 in animal models of several species, including mouse and chicken.

### Example 4

### Blocking of HUI77 Binding to Denatured-Collagen by D93

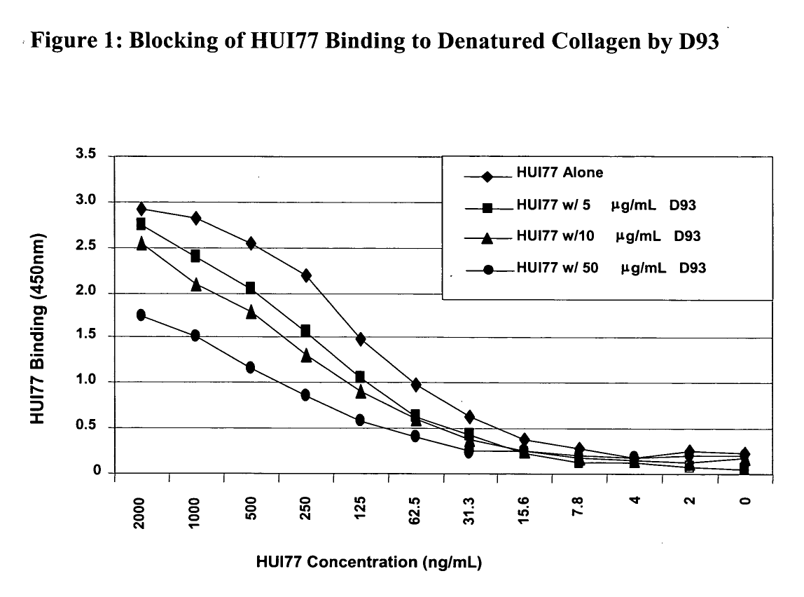

D93 was shown to compete with HUI77 for binding to human denatured-collagen IV. As shown in FIG. 1, concentration-dependent inhibition of HUI77 binding to dn-collagen IV by D93 was observed over a range HUI77 concentrations from 2 to 2,000 ng/mL. This suggests that D93 competes for the same binding region or inhibits HUI77 binding by steric hindrance. Complete inhibition of binding of HUI77 by D93 was not observed at the 50 μg/mL D93 inhibitor concentration and may be due to high avidity binding of HUI77 on dn-collagen IV.

### Example 5

### Western Blot Analysis of Trypsin-Digested Collagen IV Using D93

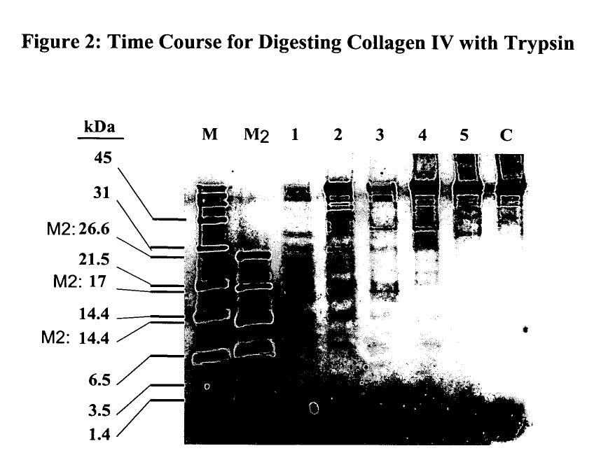

A common approach to determine antibody binding sites in a protein is to digest the target protein with an enzyme, such as trypsin, into small peptides and then identify which peptides react with the antibody (Deutzmann, R. (2004) Structural characterization of proteins and peptides. Methods Mol Med. 94, 269-297). An initial study was performed to determine the concentration of trypsin required to generate a series of peptide fragments suitable for Western blot analysis and for scale-up of peptide fragments for protein sequencing. Proteomics grade trypsin was used to prepare the fragments since it is resistant to auto-digestion. Shown in FIG. 2 is a SDS-PAGE gel of collagen IV digested with trypsin at different ratios of collagen to enzyme concentrations over an 18-hour time course. The optimum collagen/trypsin concentration ratio for generating fragments of collagen IV was determined to be 5 mg/mL collagen IV/100 μg/mL trypsin, or 50:1 (w/w), based on the range and concentration of different size fragments of collagen IV.

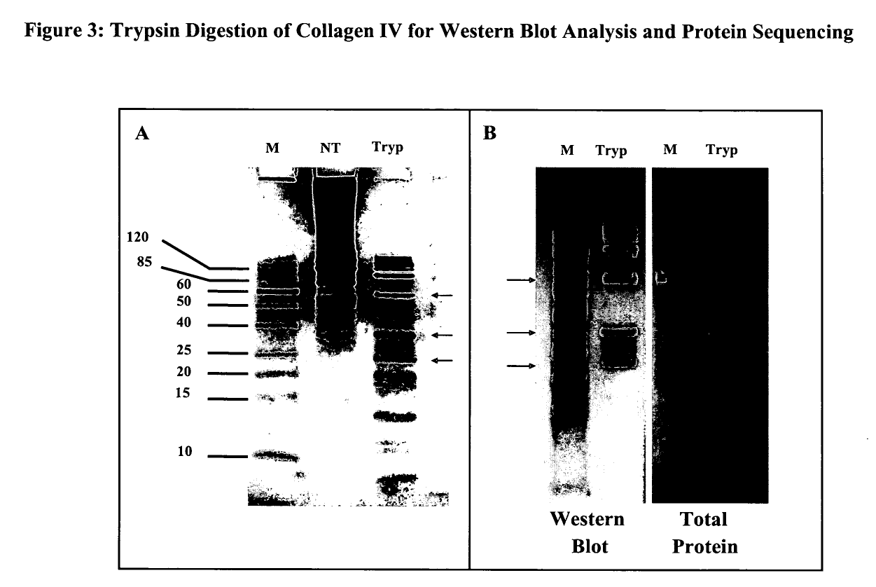

A larger quantity of trypsin-digested collagen IV was prepared based on data from the titration study shown in FIG. 2. Collagen IV (5 mg) was digested with 40 μg trypsin for 18 hours at 37° C. Analysis of collagen IV pre- and post-trypsin-digestion is shown in FIG. 3 (panel A). A sample of the trypsin-digested collagen IV contained multiple peptide fragments with a size range from less than 10 kDa to over 100 kDa. This range of peptide sizes was consistent with those observed in the titration study shown in FIG. 2. A sample of the trypsin-digested collagen IV was also subjected to SDS/PAGE with reducing agent followed by Western blot analysis using D93 antibody as a probe.

Results are shown in FIG. 3 (panel B). Western blot analysis of trypsin-digested human collagen IV shows binding of D93 to multiple collagen protein fragments. D93 bound several protein bands and had strong reactivity to protein fragments with approximate molecular weights of 23, 35, and 57 kDa (indicated by arrows). These bands have N-terminal sequences that place the binding sites at distinct regions of the triple-helical region of collagen IV suggesting that each of collagen peptide fragments contain at least one sequence that is bound by D93.

Binding of D93 to these the proteins bands suggests that the epitope(s) are a linear protein sequence since the digested collagen was subjected to SDS/PAGE in reducing conditions prior to Western analysis. The 23, 35, and 57 kDa bands were not only bound by D93, but also were well-isolated from other peptide fragments when separated by SDS-PAGE.

### Example 6

### Protein Sequencing of Collagen IV Peptides

Based on the Western blot analysis using D93 antibody, several peptides were chosen for peptide N-terminal sequencing. As shown in FIG. 3, the 23, 35, and 55 kDa were bound by D93 antibody and appeared isolated from other peptides which would reduce the chance for obtaining a mixed peptide sequence. The first peptide sequenced was the 55 kDa peptide since it had clearer separation from other peptides compared to the 23 and 35 kDa peptides. Peptide sequence information from the 23 and 35 kDa were obtained in subsequent analyses. The amino terminal sequences were determined by Edman degradation, and the results are summarized in Table 10. A clean signal was obtained for the 57 kDa peptide. The 23 and 35 kDa peptides had primary and weaker secondary signals. Some ambiguous amino acid signals were observed in the secondary signals for the 23 and 35 kDa peptides. No amino acid was detected in the third position of the secondary signal of the 23 kDa peptide. The likely reason for a blocked signal is the presence of a glycosylation or hydroxylation site on lysines (K) in collagen IV (Brown, J C and Timpl, R. (1995) “The collagen superfamily.” Int Arch Allergy Immunol. 107, 484-490; Myllyharju, J and Kivirikko, K I. (2001) Collagens and collagen-related diseases. Ann. Med. 33, 7-21).

TABLE 10
Protein Sequencing of Trypsin-digested Human Collagen IV Peptides

| COLLAGEN IV | | |
|---|---|---|
| TRYPTIC PEPTIDE | | |
| FRAGMENT | | LOCATION OF PEPTIDE |
| MOLECULAR WEIGHT | NH₂ TERMINAL SEQUENCE OF PEPTIDE | SEQUENCE |
| 23 kDa | Primary signal: | α1 (IV) G176-Hyp 190 |
| | G-F-Hyp-G-I-Hyp-G-T-Hyp-G-P-Hyp-G-L-Hyp | |
| | (SEQ ID NO: 83) | |
| | Second signal: | α1 (IV) G1064-Y1087 |
| | G-E-X-G-D-Q-G-I-A G-F-P/Hyp-G-S-Hyp | |
| | (SEQ ID NO: 84) | |
| 35 kDa | Primary signal: | α1 (IV) G1239-M1253 |
| | G-P-Q-G-Q-P-G-L-Hyp-G-L-Hyp-G-P-M | |
| | (SEQ ID NO: 85) | |
| | Second signal: | α1 (IV) G176-T183 |
| | G-F-Hyp-G-I-Hyp-T | |
| | (SEQ ID NO: 86) | |
| 57 kDa | G-D-T-G-P-Hyp-G-P-Hyp-G-Y | α1 (IV) G598-Y608 |
| | (SEQ ID NO: 87) | |

Amino acids are indicated as single letters: Hyp is hydroxyproline; a backslash indicates that other possible amino acids were detected for a given position; X is designated as no detection, which was predicted as hydroxylysine.

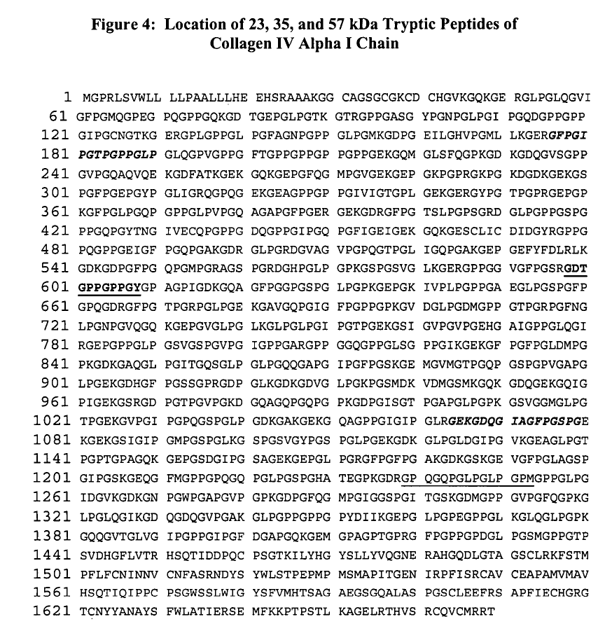

All of the amino terminal peptide sequences identified in the 23, 35, and 57 kDa collagen fragment samples shown in Table 10 were located on the alpha I chain of collagen IV. The location of the peptide sequences shown in Table 10 in the protein sequence of the alpha I chain of collagen IV are shown in FIG. 4.

### Example 7

### Screening of a Collagen IV Synthetic Peptide Array Using D93

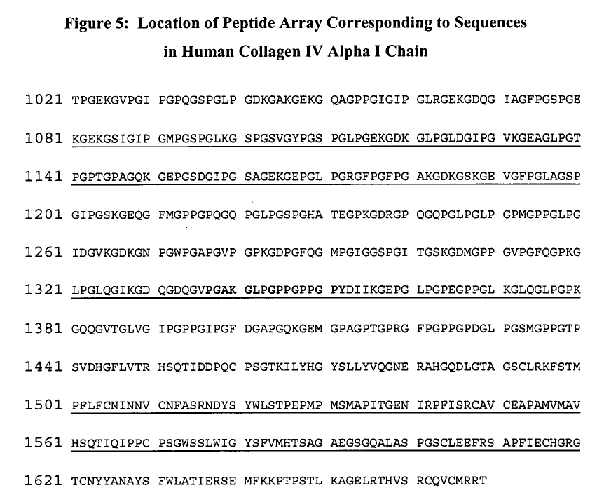

Small peptide sequences located in close proximity to peptides were identified by Edman sequencing was performed by screening a synthetic peptide array consisting of the C-terminal 1/3 of human collagen IV alpha 1 chain. This region was chosen based on the location of the N-terminal sequences of the 23 (secondary sequence) and 35 (primary) kDa peptides. The region selected for synthesis of the peptide array is shown in FIG. 5.

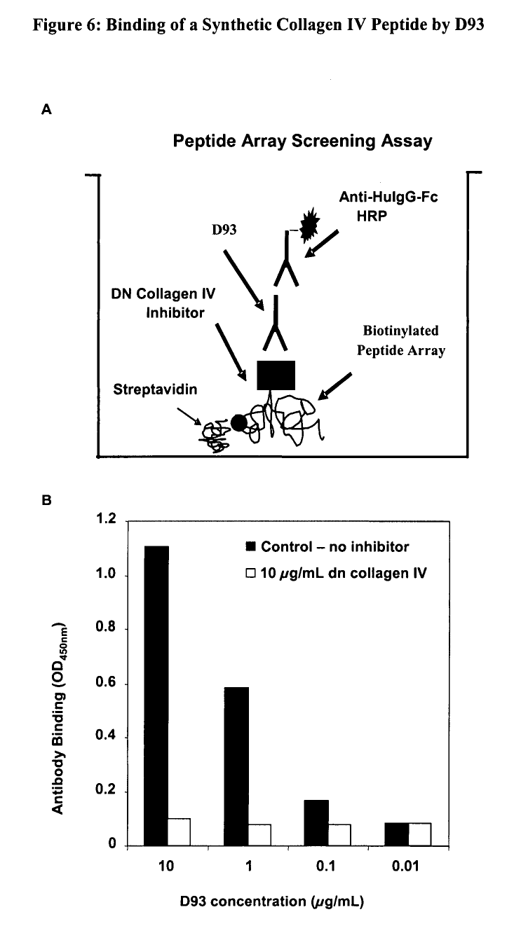

A binding assay was conducted to screen the peptide array with 10 μg/mL D93. The majority of the peptides had a low level of binding activity by D93 using conditions where D93 readily bound human heat dn-collagen IV (data not shown). Three peptides were shown to be bound by D93 with higher activity than the others and are designated as peptides 13, 40, and 58 of the peptide array. To confirm specificity of D93 binding of collagen to peptides 13, 40, and 58, D93 was pre-incubated with human heat-denatured-collagen prior to reacting to the peptides. One peptide, in the peptide array (shown in FIG. 5), showed reproducible binding by D93 and binding was inhibited by heat-denatured-collagen IV over a range of D93 concentrations (FIG. 6). Data is shown as the average of duplicate samples. Inhibition of D93 binding to the peptide by denatured-collagen suggests that the antigen-binding site of D93 is involved in binding the peptide and not the framework or constant regions of the antibody.

The core sequence of the peptide is PGAKGLPGPPGPPGPY (SEQ ID NO: 1). Several permutations of this sequence were present in the peptide array since each proline residue is a 50/50 mixture of proline and hydroxyproline. A second peptide array was synthesized to examine reactivity of D93 to individual permutations of the peptide sequence with either proline or hydroxyproline at each position. A summary of the peptides that were either bound or no bound by D93 is shown Table 11.

TABLE 11

Effect of Hydroxyproline on D93 Binding
to Synthetic Collagen Peptides

| | | MAB BINDING |
|---|---|---|
| SEO ID NO | PEPTIDE SEQUENCE | (OD_{450nM}) |
| 1 | PGAKGLPGPPGPPGPY | 0.136 |
| 3 | PGAKGLPGPPGPPGPY | 0.984 |
| 4 | PGAKGLPGPPGPPGPY | 0.328 |
| 5 | PGAKGLPGPPGPPGPY | 0.430 |
| 6 | PGAKGLPGPPGPPGPY | 0.483 |
| 7 | PGAKGLPGPPGPPGPY | 0.820 |
| 8 | PGAKGLPGPPGPPGPY | 0.847 |
| 9 | PGAKGLPGPPGPPGPY | 0.311 |
| 10 | PGAKGLPGPPGPPGPY | 1.263 |
| 11 | PGAKGLPGPPGPPGPY | 0.961 |
| 12 | PGAKGLPGPPGPPGPY | 0.313 |
| 13 | PGAKGLPGPPGPPGPY | 0.348 |
| 14 | PGAKGLPGPPGPPGPY | 0.656 |
| 15 | PGAKGLPGPPGPPGPY | 1.573 |
| 16 | PGAKGLPGPPGPPGPY | 0.182 |

P=Proline; P=Hydroxyproline. Data is represented as the average binding of D93 at 10 μg/mL in duplicate tests using the peptide array screening ELISA method. Underlined OD values indicate high binding activity.

Peptides having at least two-fold binding over the peptide of SEQ ID NO: 1 are indicative of peptides that bind to D93. Peptides that lacked or contained two adjacent hydroxyprolines had reduced D93 binding activity, thus, D93 binding to synthetic collagen peptides depends on hydroxyproline. The peptide motif G-P-Hyp-G showed increased D93 binding activity compared to peptides without hydroxyproline. D93 had the highest binding activity with a peptide having two hydroxyprolines in the motif “G-P-Hyp-G-P-Hyp” (residues 8-13 of SEQ ID NO: 15) as part of the peptide amino acid sequence. Data is represented as the average binding of D93 at 10 μg/mL in duplicate tests using the peptide array screening ELISA method. Underlined OD values indicate high binding activity.

A synthetic peptide, designated as peptide number 40, was identified by screening a 16 amino acid array of peptide sequences for inhibition of HUI77 or D93 binding to denatured-collagen IV, and had the sequence Hyp-G-A-K-G-L-P-G-P-Hyp-G-P-Hyp-G-P-Y (SEQ ID NO: 2). The amino acids correlating to increased inhibition of D93 binding to dn-collagen type IV were the motif G-P-Hyp-G-P-Hyp-G-P-Y (SEQ ID NO: 30) found in the above peptides.

The presence of hydroxyproline was required for increased inhibition of D93 binding to dn-collagen.

Inhibition of binding of D93 was observed with peptides containing the tetrapeptide motif G-X-Hyp-G but with reduced binding compared to the motif G-P-Hyp-G-P-Hyp (residues 8-13 of SEQ ID NO: 15). Some preference was associated with a P in the X position. Hydroxyproline was also required for enhanced inhibition.

A repeat of the G-X-Hyp-G sequence in the same peptide was shown to enhance the inhibition of D93 binding to dn-collagen type IV.

The G-P-Hyp-G-P-Hyp-G-P-Y (SEQ ID NO: 30) sequence was also shown to inhibit binding of HUI77 to denatured-collagen IV. The presence of hydroxyproline was required for maximum inhibition of HUI77 binding to dn-collagen IV.

The G-X-P-G and GPPGPPG (SEQ ID NO: 81) sequences are also located in multiple copies on the alpha 1 and alpha 2 chains of collagen type IV, and other collagen types, including type I, from multiple species. The presence of these sequences supports the observation that HUI77 and D93 bind to dn-collagens from multiple species by ELISA.

Since the initial peptide array contained a mixture of proline and hydroxyproline at each site, D93 was tested for binding activity to individual peptides of the P1337-Y1352 region containing homogenous sequences. As shown in Table 11, the binding of D93 to the P1337-Y1352 sequence was dependent on the presence and sequence position of hydroxyproline residues (P). Increased binding of D93 was observed with peptides containing single hydroxyproline residues compared to peptides containing a hydroxyproline-hydroxyproline dipeptide which did not bind D93 as efficiently.

Additional studies were performed to determine which amino acids of α1(IV) P1337-Y1352 were recognized by D93, and if HUI77 also bound to the same peptide sequences. Peptides of varying length within the α1(IV) P1337-Y1352 sequence were tested as inhibitors for D93 or HUI77 binding to denatured collagen IV. Based on published protein sequences, peptides were synthesized using hydroxyproline (P) at residues P1337, P1346 and P1349 of α1(IV) P1337-Y1352. As shown in Tables 13 and 14, binding of D93 to denatured collagen IV was inhibited by α1(IV) P1337-Y1352 peptide in a dose-dependent manner. A series of smaller peptides corresponding to α1(IV) L1342-Y1352 were shown to also inhibit D93 binding to denatured collagen IV at concentrations similar to peptide α1(IV) P1337-Y1352. Residues P1346 and P1349 were shown to be important for inhibiting D93 binding since the shortest peptides inhibitors where GPPG (SEQ ID NO: 34) or GPPGPPG (SEQ ID NO: 32) where P is hydroxyproline. Peptides containing residue Y1352 exhibited greater inhibition of D93 binding to denatured collagen IV. HUI77 binding to denatured collagen IV was also inhibited by the same panel of synthetic peptides, although a higher concentration of peptides was required for inhibiting HUI77 binding compared to D93.

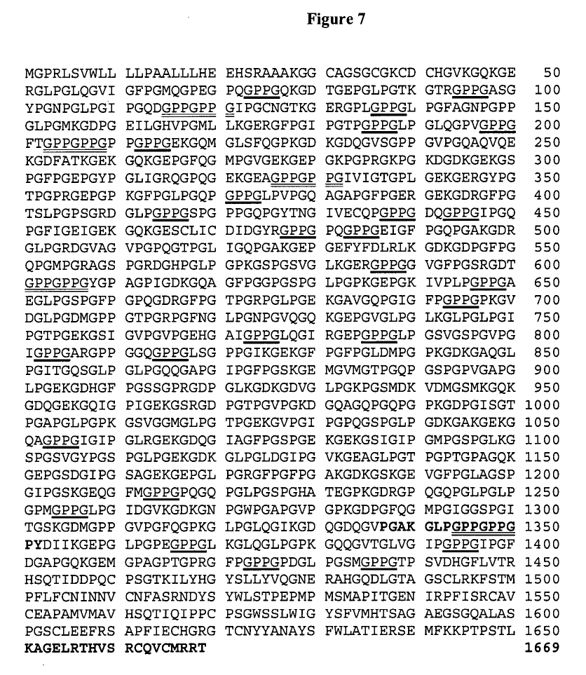

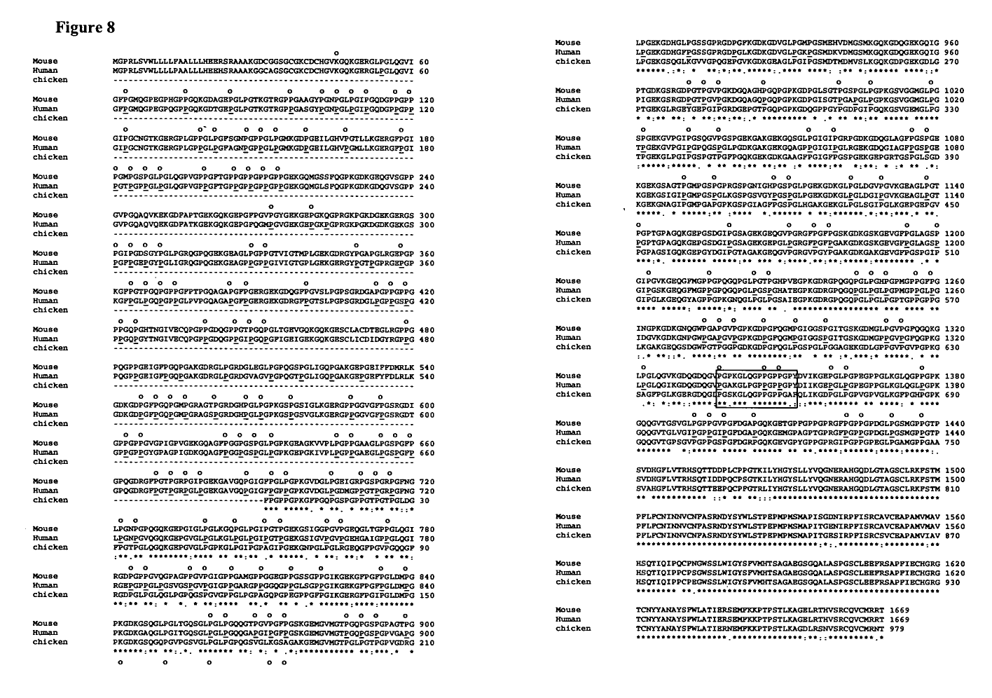

The location of GPPG (SEQ ID NO: 35) and GPPGPPG (SEQ ID NO: 81) peptide sequences within the helical domain of the al chain of collagen IV are shown in FIG. 7 and FIG. 8. The number of repeats and their location support the observation that D93 is binding to 23-, 35- and 57-kDa tryptic peptides. These sequences are also highly repeated in collagens from multiple species, including chicken, human and mouse (FIG. 8), which explains the anti-angiogenic activity of D93 in models based on these species (Pernasetti, F. et al., Int. J. Oncol. 2006, December; 29(6): 1371-1379). In addition, single or multimerized GPPG sequences are present in numerous copies in many human collagen types as shown in Table 12, which supports the observation that D93 and HUI77 are binding to collagen types I through V (Xu, J. et al., (2001) J. Cell Biol. 154, 1069-1079; Xu, J. et al., (2000) Hybridoma 19, 375-385; and Pernasetti, F. et al., Int. J. Oncol. 2006, December; 29(6): 1371-1379). The number of potential collagen binding sites for D93 also extends beyond the GPOG sequence since D93 binding to denatured collagen IV was also inhibited by peptides containing the tetramer sequence GXOG, where X could be P, L, F or T (data not shown).

TABLE 12
Multiple D93 and HU177 Binding Sites
on Different Human Collagen Chains

| Collagen | | | Number of GPPG |
|---|---|---|---|
| Type and | Sequence | Number of GPPG | Multimer Sequences |
| Chain | Reference¹ | Sequences Per Chain² | Per Chain² |
| α1(I) | P02452 | 26 | 7 |
| α2(I) | P08123 | 18 | 5 |
| α1(II) | P02458 | 24 | 4 |
| α1(III) | Q8N6U4 | 18 | 6 |
| α1(IV) | P02462 | 26 | 5 |
| α2(IV) | P08572 | 25 | 3 |
| α1(V) | P20908 | 24 | 8 |

¹References for sequences are accession numbers from the “pir.georgetown.edu website.
²Single and multimerized GPPG sequences were obtained by scanning the collagen chain sequences using the listed accession numbers. The presence of hydroxyproline was not determined since the databases lacked sufficient information for all chains.

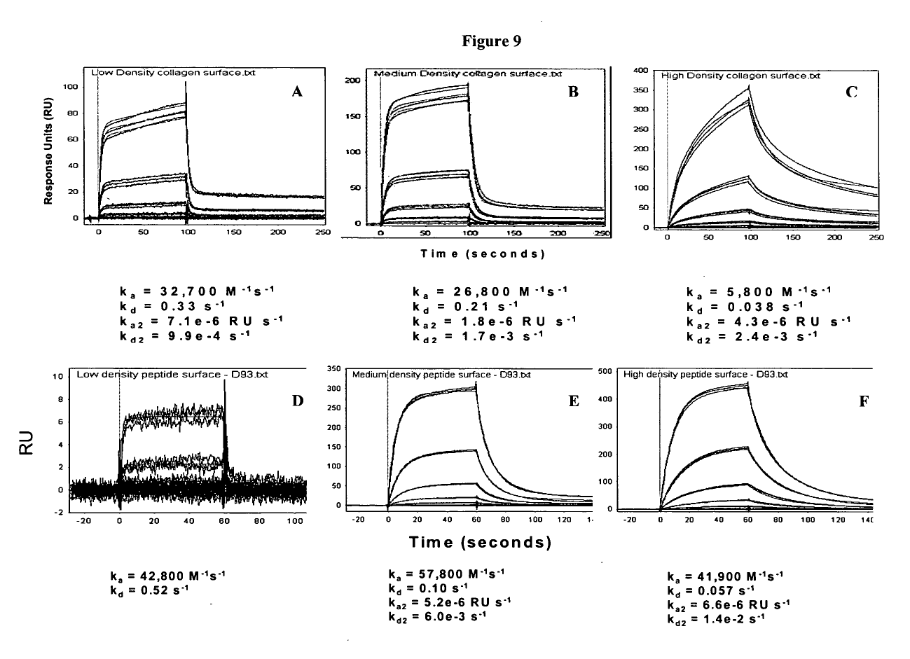

A D93 binding signal was obtained with three different densities of dn-collagen IV coupled to a plasmon resonance sensor chip, and with a synthetic collagen peptide representing a region containing a D93 epitope. Binding activity positively correlated with increased D93 antibody concentration in solution and increasing surface density of dn-collagen. Triplicate determinations showed the reproducibility of measurements for each of the D93 antibody and dn-collagen concentrations. The low, medium, and high chip surface density of dn-collagen showed for monomeric (one Fab region) binding of D93 an equilibrium dissociation constant (KD) of 10 μM, 7.8 μM, and 6.5 μM, respectively (FIG. 9). The low, medium, and high chip surface density of a biotinylated collagen peptide α1(IV) corresponding to sequence position P1337-Y1352 revealed a KD for the monomeric D93 Fab of 11 μM, 1.7 μM, and 1.3 μM, respectively.

Using sensor chips with a low or medium density of dn-collagen or coated with synthetic peptide, KD values for bivalent antibody binding were obtained with values between 30 nM and 63 nM. No binding of control antibody DP28 to dn-collagen or biotinylated synthetic collagen peptide was detectable (data not shown).

Specific binding sites for D93 (and parental mAb HUI77) on collagen type IV have been identified. These binding sites are cryptic because they are exposed on subendothelial basement membranes from tumors and normal tissue undergoing neovascularization, but not on membranes from normal tissues (Pernasetti et al. (2006) Int. J. Oncol. 29(6): 1371-1379).

GPHyp repeat motifs of collagens were recognized as binding epitopes for humanized antibody D93. Binding of D93 to cryptic sites of collagen IV may occur via a change in conformation of triple-helical collagen following proteolysis or other denaturation, or via the exposure of otherwise hidden epitopes. Crystal structures of collagen peptides containing GPHyp repeats showed that the hydroxylate imino group of 4-hydroxyproline residues are directed to the outside of the triple helix (Bella et al. (1994) Science, 266: 75-81), where they may be involved in intra- and inter-chain bonding with adjacent triple helixes through hydrogen bonding with water. D93 may, therefore, be binding to un-tethered hydroxyproline residues of GPHyp sequences of collagen chains or peptide fragments that are no longer participating in intra- or inter-chain interactions as a consequence of, for example, thermal denaturation or proteolytic degradation by enzymes such as MMPs. In the present application, D93 was shown to bind a synthetic collagen peptide containing a tandem GPHypGPHyp sequence corresponding to α1 (IV) P1337-Y1352 located in the C-terminal region of the triple-helical domain homologous in sequence and location to proposed nucleation sites of collagen I.

Affinity measurement for D93 binding to denatured collagen and the synthetic α1 (IV) P1337-Y1352 peptide containing hydroxyprolines in the natural sequence was shown to be similar over a range of different target densities. The affinity of D93 for both denatured collagen and synthetic peptides was directly proportional to target density. An increase in bivalent interaction of both D93 Fab regions, therefore, likely results in an increased avidity for the target binding sites. Multi-valent binding may, in this way, increase the specificity of D93 for denatured collagen which is largely present in areas of neo-vascularization or matrix remodeling, but not in established vessels present in healthy tissues, where slow turnover of collagen occurs. Therefore, in vivo binding of D93 to cryptic epitopes such as those identified in the present application to blood vessels of tumor sites has therapeutic applications (Pernasetti et al. (2006) Int. J. Oncol. 29(6): 1371-1379).

### Example 8

### Inhibition of D93 Binding to Denatured-Collagen IV by Specific Amino Acid Deletions and/or Substitutions in Synthetic Collagen Peptides

A series of peptides was synthesized to determine the specific amino acid residues within the peptide sequence PGAKGLPGPPGPPGPY (SEQ ID NO: 1) that targets the D93 binding. These sequences showing the greatest D93 binding activity contained hydroxyproline residues in the correct positions in the collagen IV alpha I chain as referenced in the Protein Information Database at the website “pir.georgetown.edu.” In addition, specific substitutions were made in peptides that closely matched the sequence of the peptide and were represented at least once in the collagen IV alpha I chain. Additional peptides were synthesized without the N-terminal biotin and used inhibitors at 1,000, 100, 10, and 1 μM in an ELISA to block D93 binding to heat-denatured-collagen IV. Shown in Table 13 is data reported as the % inhibition of D93 binding to human denatured-collagen compared to 1% BSA/PBS alone. A non-collagen peptide, CTWPRHHTTDALL (SEQ ID NO: 39), was used as a control for non-specific inhibition of D93 binding.

Maximum levels of inhibition of binding of D93 to denatured-collagen IV were observed at 1000 μM and 100 μM of peptide Hyp-G-A-K-G-L-P-G-P-Hyp-G-P-Hyp-G-P-Y (SEQ ID NO: 2) and decreased to 81% and 30% at 10 μM and 1 μM, respectively. The blocking activity of this peptide was located in C-terminal 9 amino acids with the sequence G-P-Hyp-G-P-Hyp-G-P-Y (SEQ ID NO: 30) and had inhibitory activity similar to that observed with the peptide. The presence of the terminal Y residue in SEQ ID NO: 30 further enhanced this inhibitory activity compared to the peptide of SEQ ID NO: 31 in which the terminal Y residue is not present. D93 blocking activity was dependent on the presence of hydroxyproline in the known positions referenced in the collagen sequence. A smaller peptide with the sequence G-P-Hyp-G (SEQ ID NO: 34) also showed D93 blocking activity, but only at the 1000 μM and 100 μM concentrations. Similarly, the presence of hydroxyproline in the peptide enhanced inhibition of the D93 binding. The addition of L-Hyp to the G-P-Hyp-G sequence (SEQ ID NO: 34) enhanced the D93 blocking activity at 1000 μM from 52% to 90%. Substitution of P in X position of the sequence L-Hyp-G-X-Hyp-G (SEQ ID NO: 90) had greater D93 blocking activity compared to T, S, L, and F when tested at 1000 μM. Multiple repeats of the G-P-Hyp-G sequence (SEQ ID NO: 34), as in the sequence F-Hyp-G-P-Hyp-G-P-D-G-L-P-G-S-M-G-P-Hyp-G (SEQ ID NO: 36), had increased D93 blocking activity compared to a single G-P-Hyp-G sequence (SEQ ID NO: 34).

TABLE 13

Identification of Specific Amino Acids of Synthetic Peptide that
Inhibit Binding of D93 to Human Denatured-collagen IV

| | |
|---|---|
| | % INHIBITION OF BINDING OF D93 TO |
| | HUMAN DENATURED-COLLAGEN IV |
| PEPTIDE ID | PEPTIDE INHIBITOR |
| NO. (SEQ ID | CONCENTRATION |

| | | | | | |
|---|---|---|---|---|---|
| NO.) | PEPTIDE SEQUENCE | 1000 μM | 100 μM | 10 μM | 1 μM |

| | | | | | |
|---|---|---|---|---|---|
| 12 (2) | PGAKGLPGPPGPPGPY | 93.5 | 93.5 | 81 | 30 |
| 13 (17) | PGAKGLPGP | 11.5 | 4 | 6 | 11 |
| 2 (18) | LPGPPGPPGPY | 46 | 7 | 2 | 6 |
| 11 (19) | LPGPPGPPGPY | 93.5 | 93.5 | 83 | 32 |
| 5 (20) | LPGPPGPPGP | 92.5 | 89 | 41.5 | 12 |
| 20 (21) | LPGPPGPPG | 92.5 | 85.5 | 35.5 | 13 |
| 18 (22) | LPGPPGPPG | 17.5 | 3 | 5 | 6 |
| 19 (23) | LPGFPG | 32 | 8 | 6 | 10.5 |
| 16 (24) | LPGPPG | 90 | 52.5 | 12 | 6.5 |
| 14 (25) | LPGLPG | 46.5 | 6.5 | 6.5 | 8 |
| 10 (26) | LPGSPG | 13.5 | 2.5 | 3.5 | 9.5 |
| 8 (27) | LPGTPG | 24 | 4.5 | 2 | 7 |
| 9 (37) | LPGFPGC | 6.5 | 1 | 3.5 | 8 |
| 24 (38) | LPGFPG | 11.6* | 8.3* | 7.8* | 5.4* |
| 4 (28) | PGPPGPPGPY | 93.5 | 93.5 | 83 | 33.5 |
| 1 (29) | PGPPGPPGP | 93.5 | 87.5 | 40.5 | 11.5 |
| 3 (30) | GPPGPPGPY | 94 | 94, 90.3* | 82.5, | 33 |
| | | | | 79.9* | |
| 17 (31) | GPPGPPGP | 92.5 | 77 | 21 | 9.5 |
| 15 (32) | GPPGPPG | 92.5 | 64 | 17 | 8 |
| 23 (33) | GPPGPPG | 6.5 | 1.5 | 4.5 | 5 |
| 21 (34) | GPPG | 52 | 11 | 6.5 | 9.5 |
| 22 (35) | GPPG | 7.5 | 3.5 | 6 | 7 |
| 7 (36) | FPGPPGPDGLPGSMGPPG | 93.5 | 89.5 | 43 | 11 |
| 6 (39) | CTWPRHHTTDALL | 3.5, 4.9* | 0 | 1 | 4.5 |

Data shown is the average percent inhibition of D93 (prepared by AppTec; 12 mg/mL) binding to denatured-collagen from two studies. Each peptide concentration was tested in triplicate in each study. D93 and peptide inhibitor were pre-incubated for 15 minutes at room temperature prior to adding to microtiter plate wells coated with denatured-collagen. P indicates hydroxyproline at that amino acid residue. Asterisk (*) indicates data was obtained from a separate assay. Percentage of (%) inhibition of binding of D93 to denatured collagen IV of at least 2-fold greater than negative controls (i.e., CTWPRHHTTDALL (SEQ ID NO: 39), LPGFPG (SEQ ID NO: 38), and LPGFPGC (SEQ ID NO: 37)) is indicative of a peptide that inhibits binding of D93 to human denatured collagen IV. Percentage (%) of inhibition of binding of D93 to denatured collagen IV of less than 2-fold greater than negative controls (i.e., CTWPRHHTTDALL (SEQ ID NO: 39), LPGFPG (SEQ ID NO: 38), and LPGFPGC (SEQ ID NO: 37)) is indicative of a peptide that does not inhibit binding of D93 to human denatured collagen IV.

### Example 9

### Inhibition of HUI77 Binding to Denatured-Collagen IV by Specific Amino Acid Deletions and/or Substitutions in Synthetic Collagen Peptides

In FIG. 1, D93 was shown to block binding of HUI77 to dn-collagen IV. Since the D93 antibody was derived by humanization of the murine monoclonal antibody HUI77, it seemed likely that similar synthetic peptides that blocked the binding of D93 (as shown in Table 13 above) would also block the binding of HUI77 to dn-collagen IV in a similar manner. Peptide blocking of HUI77 was performed using the same peptides that are shown in Table 13, and the results are shown Table 14. HUI77 binding to denatured-collagen was inhibited by the sequence Hyp-G-A-K-G-L-P-G-P-Hyp-G-P-Hyp-G-P-Y (SEQ ID NO: 2) by 86% at 1000 μM. The inhibitory activity of this peptide was also located in the C-terminal 9 amino acids with the sequence G-P-Hyp-G-P-Hyp-G-P-Y (SEQ ID NO: 30) and had similar inhibitory activity compared to the larger 16-mer peptide at each of the concentrations tested. Inhibition of HUI77 binding to denatured-collagen by these peptides, as with D93, was also dependent on the presence of hydroxyproline. Only partial inhibition of HUI77 binding to denatured-collagen was demonstrated using 1000 μM peptides tested compared to the maximum inhibition of D93 at both 1000 μM and 100 μM concentrations for the same peptides. This is likely due to a lower binding affinity of HUI77 to the peptides as compared to D93. Similar to D93, removal of the C-terminal Y on peptide 40 reduced the inhibition of HUI77 binding to denatured-collagen (compare to Table 13). In contrast to D93, the sequence L-Hyp-G-F-Hyp-G (SEQ ID NO: 23) had similar inhibitory activity compared to L-Hyp-G-P-Hyp-G (SEQ ID NO: 24) when blocking HUI77 binding to denatured-collagen (compare to Table 13).

TABLE 14

Synthetic Peptides Used for Inhibition of
HU177 Binding to Denatured-collagen IV

| | | |
|---|---|---|
| | | % INHIBITION OF BINDING OF |
| | | HUI77 TO DENATURED |
| | | COLLAGEN IV |
| | | PEPTIDE INHIBITOR |
| (SEQ ID | MOLECULAR | CONCENTRATION |

| | | | | | |
|---|---|---|---|---|---|
| NO) | WEIGHT | PEPTIDE SEQUENCE | 1000 μM | 100 μM | 10 μM |

| | | | | | |
|---|---|---|---|---|---|
| 12 (2) | 1508 | PGAKGLPGPPGPPGPY | 86.5 | 18 | 7.5 |
| 13 (17) | 809 | PGAKGLPGP | 10 | 10.5 | 8 |
| 2 (18) | 1048 | LPGPPGPPGPY | 4.5 | 6.5 | 5.5 |
| 11 (19) | 1081 | LPGPPGPPGPY | 87 | 18.5 | 6.5 |
| 5 (20) | 918 | LPGPPGPPGP | 73 | 14 | 10 |
| 20 (21) | 821 | LPGPPGPPG | 86 | 18 | 6.5 |
| 18 (22) | 788 | LPGPPGPPG | 6 | 9 | 6.5 |
| 19 (23) | 619 | LPGFPG | 75 | 14.5 | 7 |
| 16 (24) | 569 | LPGPPG | 64 | 8 | 3 |
| 14 (25) | 584 | LPGLPG | 17.5 | 9.5 | 6 |
| 10 (26) | 558 | LPGSPG | 8.5 | 10.5 | 8 |
| 8 (27) | 572 | LPGTPG | 6 | 4.5 | 3.5 |
| 9 (37) | 690 | LPGFPGC | 8.5 | 7.5 | 7 |
| 24 (38) | 588 | LPGFPG | 3.7* | 0* | 10.2* |
| 4 (28) | 968 | PGPPGPPGPY | 86.5 | 16 | 7 |
| 1 (29) | 804 | PGPPGPPGP | 63 | 12.5 | 10.5 |
| 3 (30) | 871 | GPPGPPGPY | 78, 63* | 12, 0* | 8.5 |
| 17 (31) | 707 | GPPGPPGP | 44 | 12 | 7.5 |
| 15 (32) | 610 | GPPGPPG | 77 | 7.5 | 6 |
| 23 (33) | 579 | GPPGPPG | 1 | 2 | 1.5 |
| 21 (34) | 343 | GPPG | 14 | 9 | 6.5 |
| 22 (35) | 326 | GPPG | 3.5 | 8 | 2.5 |
| 7 (36) | 1699 | FPGPPGPDGLPGSMGPPG | 62 | 9 | 4.5 |
| 6 (39) | 1551 | CTWPRHHTTDALL | 6.5, 11.1* | 6.5 | 6.5 |

Data shown is the average percent inhibition of HUI77 binding to denatured-collagen from two studies. Each peptide concentration was tested in triplicate for each study. HUI77 and peptide inhibitor were pre-incubated for 15 minutes at room temperature prior to adding to microtiter plate wells coated with denatured-collagen. P indicates hydroxyproline at that amino acid residue. Asterisk (*) indicates data was obtained from a separate assay. Percentage of (%) inhibition of binding of HUI77 to denatured collagen IV of at least 2-fold greater than negative controls (i.e., CTWPRHHTTDALL (SEQ ID NO: 39), LPGFPG (SEQ ID NO: 38), and LPGFPGC (SEQ ID NO: 37)) is indicative of a peptide that inhibits binding of HUI77 to human denatured collagen IV. Percentage (%) of inhibition of binding of HUI77 to denatured collagen IV of less than 2-fold greater than negative controls (i.e., CTWPRHHTTDALL (SEQ ID NO: 39), LPGFPG (SEQ ID NO: 38), and LPGFPGC (SEQ ID NO: 37)) is indicative of a peptide that does not inhibit binding of HUI77 to human denatured collagen IV.

### Example 10

### Homology of D93 Binding Sequences of Collagen IV Alpha I Chain of Different Species and of Other Collagen Types

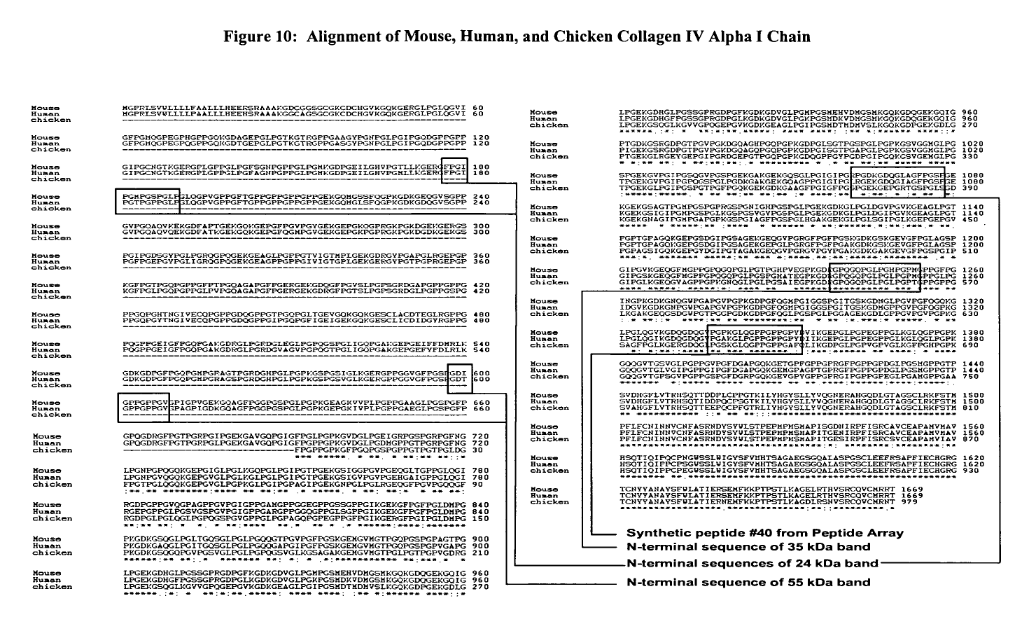

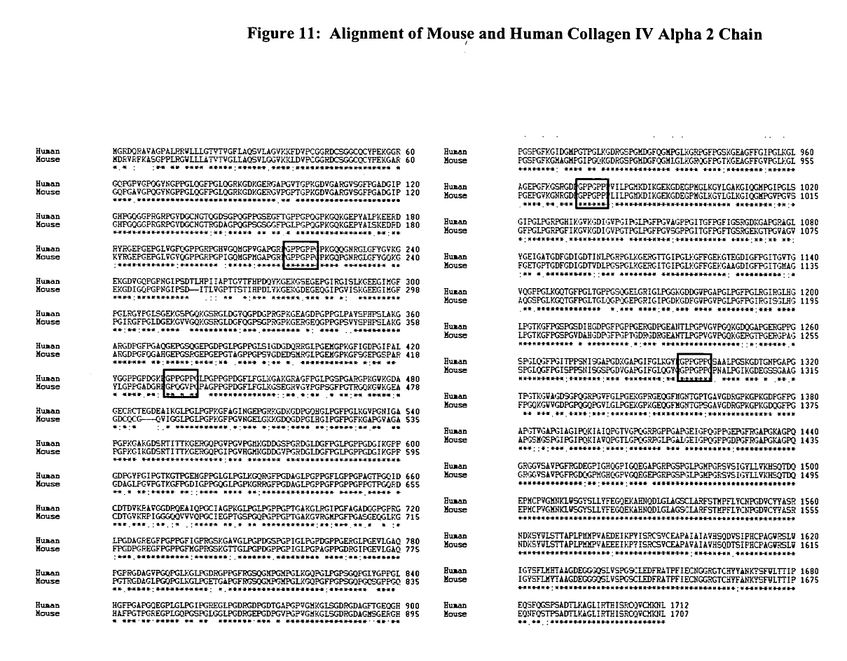

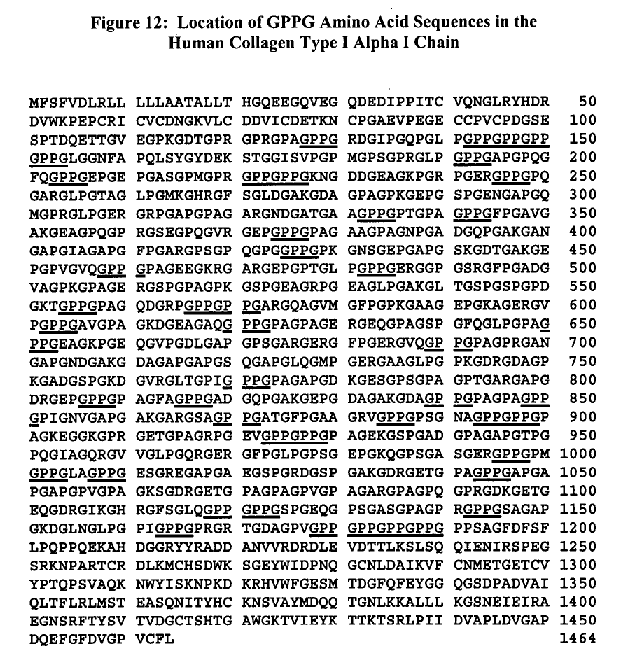

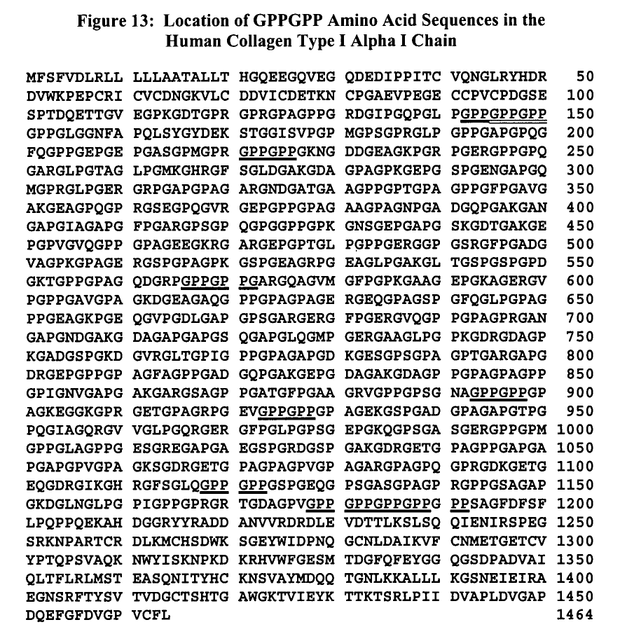

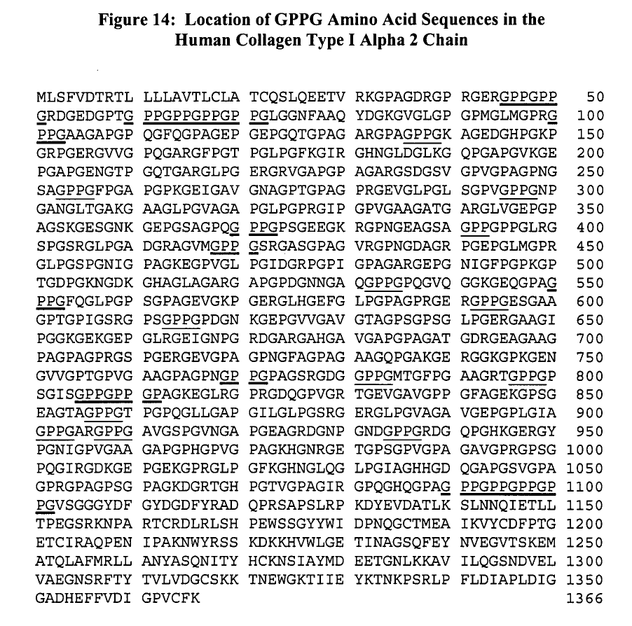

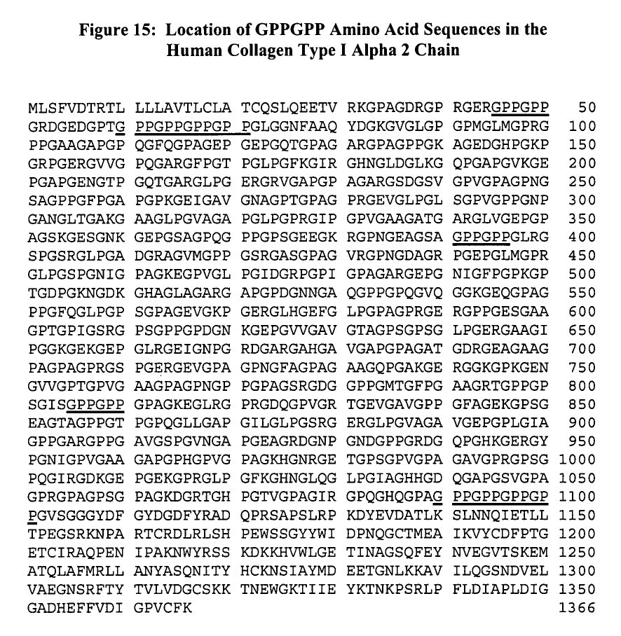

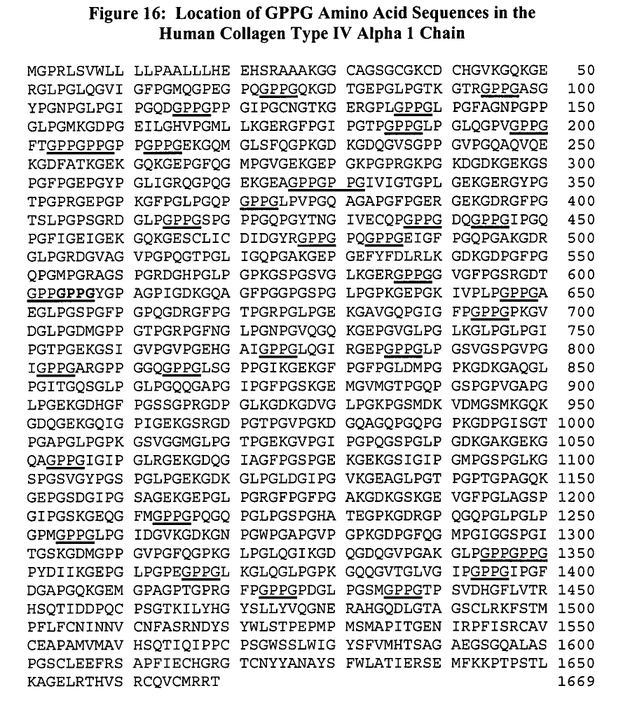

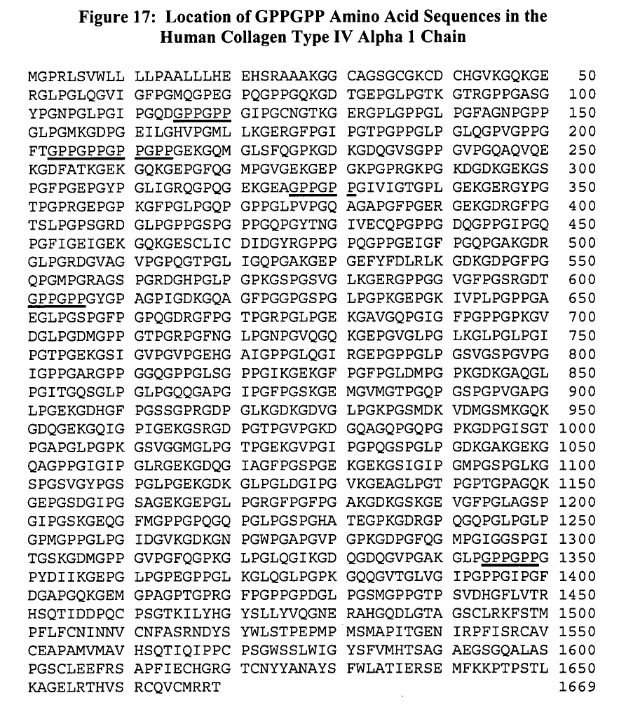

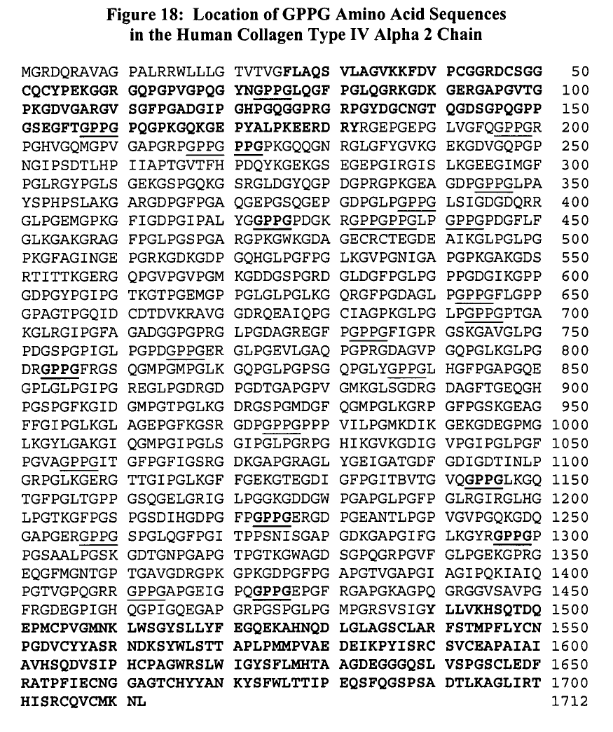

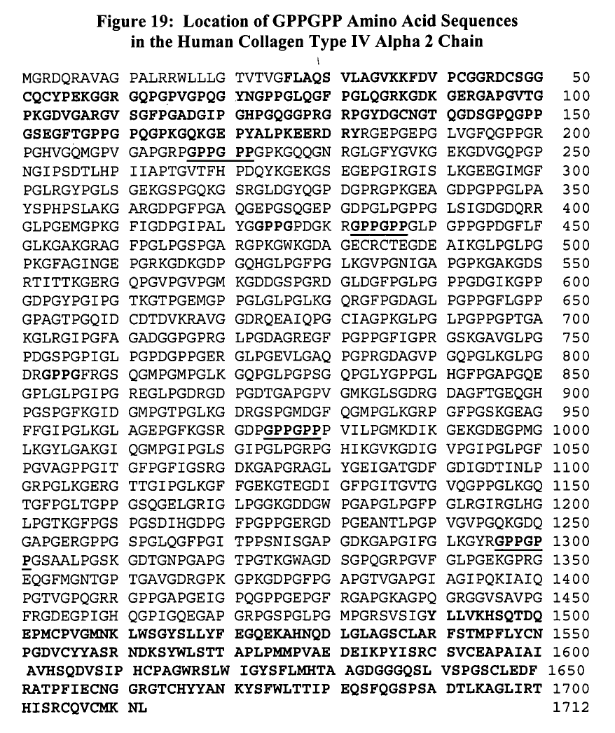

D93 and HUI77 have been shown to bind mouse and human collagen types I and IV and to have anti-angiogenic properties as characterized in vivo by a CAM assay. An alignment of amino acid sequences of mouse, chicken and human collagen is shown in FIG. 10. The presence of the sequence G-P-P-G-P-P (SEQ ID NO: 81) is observed in all three species and in multiple locations. Similar sequences are observed in the collagen IV alpha 2 chain of humans and mice as shown in FIG. 11. In addition, similar sequences are located on both the alpha 1 and alpha 2 chain of human collagen type I. Thus the reactivity of D93 and the parent murine antibody, HUI77, for collagens I, II, III, IV, and V is likely due to a conserved peptide sequence similar in structure to G-X-Y-G where the preferred sequence for X is P for reactivity to D93 and Y is hydroxyproline. Multimers of the G-X-Y-P sequence either adjacent to one another or within short peptide stretches are present in multiple copies in both the alpha 1 and 2 chains of collagen types I and IV. The location of the G-P-P-G (SEQ ID NO: 35) and G-P-P-G-P-P (SEQ ID NO: 81) sequences on the alpha 1 and alpha 2 chains of collagen types I and IV are shown in FIG. 12, FIG. 13, FIG. 14, FIG. 15, FIG. 16, FIG. 17, FIG. 18 and FIG. 19.

### Example 11

### Antagonists Inhibit Endothelial Cell Adhesion and Migration

This example demonstrates that certain antagonists of the invention can inhibit human endothelial cell adhesion to denatured collagens.

HUI77 showed the capacity to inhibit human endothelial cell adhesion to denatured collagen type-I by approximately 40% as compared to control antibody (data not shown). These findings suggest that HUI77 binds to a cryptic epitope within collagen type-I that is at least partially involved in endothelial cell adhesion to denatured collagen-I. Since endothelial cell adhesive processes are thought to play a role in tumor growth and angiogenesis, this function blocking antibody may have an effect on angiogenesis and tumor growth in vivo.

HUI77 also showed the capacity to inhibit human endothelial cell migration on denatured collagen-I by approximately 80% as compared to either control antibody or no treatment (data not shown). These findings suggest that HUI77 binds to a cryptic epitope within collagen type-I that plays a significant role in cellular migration on denatured collagen I. Given that cell migration is thought to play an important role in tumor metastasis and angiogenesis, and that denatured collagen was detected in association with malignant tumor cells and angiogenic blood vessels, this function blocking antibody may have a significant impact on angiogenesis and tumor growth and metastasis in vivo.

### Example 12

### Inhibition of Angiogenesis by HUI77

This example shows that antagonists of the invention effectively inhibit angiogenesis in a chick CAM assay.

Systemic administration of HUI77 inhibited βFGF induced angiogenesis by approximately 90% as compared to controls (data not shown). Angiogenic index was measured by counting the number of blood vessel branch points in the chick CAM assay. Importantly, no toxic side effects were noted in the embryos during the assay period. Moreover, few if any effects from HUI77 were noted on normal quiescent blood vessels

### Example 13

### Inhibition of Tumor Growth by HUI77

This example shows that antagonists of the invention effectively inhibit tumor growth in melanoma tumors in vivo.

Systemic administration of HUI77 inhibited Melanoma tumor growth by approximately 53% as compared to controls (data now shown). Importantly, no toxic side effects were noted in the embryos during the assay period. Moreover, little if any effects from HUI77 were noted on adjacent tissue. These findings indicate that HUI77 is a potent anti-tumor reagent.

### Example 14

### Effect of Monoclonal Antibodies D93 in Orthotopic Human Breast Tumor Model

The effect of antibodies D93 in an orthotopic human breast tumor model was evaluated. MDA-MB-435 tumor cells (0.4×10⁶ cells/mouse) in 50 μl PBS were orthotopically implanted in the mammary fat pad of female nude mice (five to six weeks old). When tumors reached a mean volume of approximately 50-80 mm³, mice were randomized (at least 10/group) and intravenous treatment with D93 at 1 μg (0.05 mg/kg) per dose, 10 μg (0.5 mg/kg), 100 μg (5 mg/kg) or 200 μg (10 mg/kg), or 100 μg control antibody DP28 in 100 μl PBS, or vehicle PBS 100 μl twice per week was initiated; in some studies, an untreated group was also evaluated.

| Treatment Groups | Animals/Group |
|---|---|
| 1. PBS | 12 |
| 2. D93 100 μg i.v. 2×/week | 12 |
| 3. D93 10 μg i.v. 2×/week | 12 |
| 4. D93 1 μg i.v. 2×/week | 12 |
| 5. DP-28 (Control Ab) 100 μg i.v. 2×/week | 12 |

Tumor sizes were measured every 7-20 days starting at the first day of treatment and continued until day 50-80 post-treatment. Tumor (L) and width (W) were measured with calipers and tumor volumes were calculated using the formula; (L×W²)/2, where L was the length of the tumor or the largest diameter, and W was the width or shortest diameter. Animals were euthanized at the end of the study and final tumor measurements were recorded.

TABLE 15
Percent Inhibition of Tumor Growth by D93 vs. Controls
in Orthotopic MDA-MB-435 Human Breast Tumor Model
in Mice in Three Independent Studies

| Study | Dose | % Inhibition vs. PBS | | % Inhibition | |
|---|---|---|---|---|---|
| Number | (μg/dose) | Control | | vs. DP28 | |
| A | 200 | 36% | (p < 0.05) | Not applicable | |
| | 100 | 35% | (p < 0.05) | Not applicable | |
| B | 100 | 39% | (ns) | 43% | (p < 0.05) |
| | 10 | 17% | (ns) | 22% | (ns) |
| | 1 | 0% | (ns) | 0.5% | (ns) |
| C | 100 | 52% | (p < 0.05) | 52% | (p < 0.05) |
| | 10 | 20% | (ns) | 20% | (ns) |
| | 1 | 17% | (ns) | 17% | (ns) |

Table 15 summarizes D93 dose evaluated and percent inhibition of tumor growth compared to the specified control in three independent experiments. The control used in studies B and C, DP28, was a humanized IgG₁k antibody constructed from D93 that was evaluated as a negative control for tumor model investigations. DP28, although structurally similar to D93, does not bind to denatured or native collagens. D93 at 100 μg/dose (5 mg/kg) administered twice weekly inhibited tumor growth 35-52% compared to the PBS control and approximately 43-52% compared to the DP28 monoclonal antibody control.

TABLE 16
Tumor Volumes Measured in Three Independent Studies
Using Orthotopic MDA-MB-435 Human Breast Tumor
Model in Mice after Treatment with D93

| | Day | | | | | | |
|---|---|---|---|---|---|---|---|
| | 1 | 12 | 28 | 42 | 53 | 70 | 74 |
| PBS | 57 | 103 | 329 | 524 | 966 | 1648 | 1805 |
| D93-100 | 57 | 104 | 237 | 394 | 662 | 842 | 872 |
| D93-10 | 56 | 132 | 291 | 487 | 789 | 1400 | 1451 |
| D93-1 | 56 | 124 | 334 | 654 | 952 | 1420 | 1507 |
| DP-28 | 57 | 147 | 393 | 563 | 976 | 1675 | 1815 |

As indicated in Table 16, although D93 at 10 or 1 μg/dose twice per week did not significantly inhibit tumor growth, a dose-dependent effect was observed in two studies since percent inhibition was greatest for 100, then 10 and 1 μg/dose. No difference in percent inhibition of tumor growth was observed for mice treated with 100 or 200 μg/dose administered twice per week over the dosing period, indicating that the maximum inhibitory effect was achieved at 100 μg/dose administered twice per week.

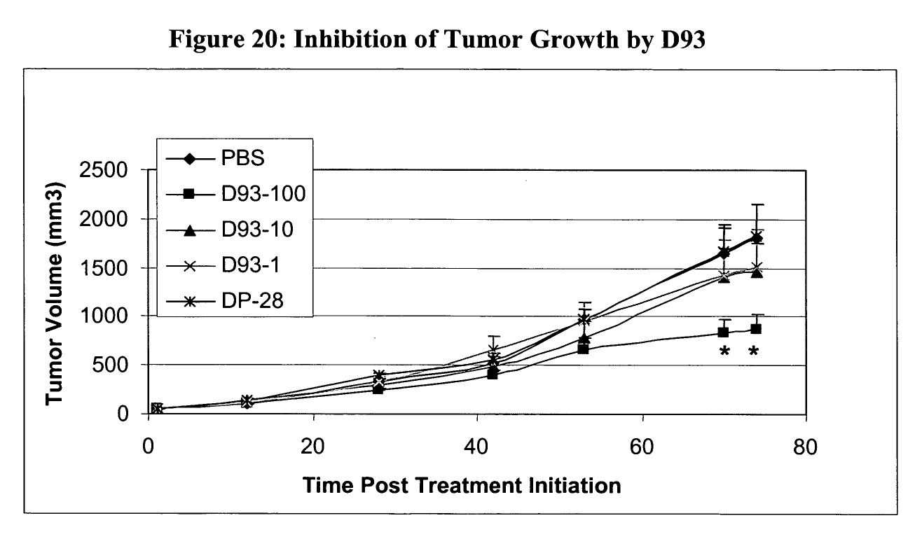

Table 16 shows the tumor volume data calculated for tumor growth over time after treatments of up to 100 μg of D93 (FIG. 20). The effect of D93 on tumor growth was statistically significant as compared to control groups.

Deposit Information

A deposit of the monoclonal antibodies HUI77 disclosed above has been made with the American Type Culture Collection (ATCC), 10801 University Boulevard, Manassas, Va. 20110. The date of deposit of monoclonal antibody HUI77 was Feb. 2, 2005. HUI77 was assigned PTA-6551.

The deposits will be maintained in the depository for a period of 30 years, 5 years after the most recent request for a sample, or for the effective life of the patent, whichever is longer, and will be replaced as necessary during that period.

What is claimed is:

1. An antagonist that preferentially binds to a binding site on a denatured collagen, wherein said binding site consists essentially of, a polypeptide having an amino acid sequence set forth as SEQ ID NO: 81 (GPPGPP) wherein one or more proline residues is hydroxyproline.

2. The antagonist of claim 1, wherein said antagonist is an antibody or functional fragment thereof.

3. The antagonist of claim 2, wherein said antibody, or functional fragment thereof, is selected from among a polyclonal antibody, a monoclonal antibody, a chimeric antibody, a humanized antibody, a human antibody, a labeled antibody, a Fab, a F(ab)₂, a F(ab′)₂, a scFv and a genetically engineered antibody.

4. The antagonist of claim 1, wherein said antagonist inhibits angiogenesis.

5. The antagonist of claim 1, wherein said antagonist prevents, inhibits, or treats an angiogenesis-dependent disorder, a cell proliferative disorder or a collagen-dependent disorder.

6. A composition comprising a pharmaceutically acceptable carrier or excipient and an antagonist of claim 1.

7. The composition of claim 6, further comprising a moiety selected from among a therapeutic moiety, an imaging moiety, a diagnostic moiety and a combination thereof.

8. The composition of claim 6, wherein the composition is substantially free of pyrogens.

9. A pharmaceutical package or kit comprising a composition of claim 6 and one or more packaging materials.

10. The pharmaceutical package or kit of claim 9, further comprising a label indicating use of the composition for inhibiting angiogenesis or an angiogenesis-dependent disease or disorder.

11. The pharmaceutical package or kit of claim 10, wherein the angiogenesis-dependent disease or disorder is selected from among ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and haemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

12. The pharmaceutical package or kit of claim 9, further comprising instructions for use.

13. An antagonist that preferentially binds to a binding site on a denatured collagen, wherein said binding site consists essentially of an isolated polypeptide having an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

14. The antagonist of claim 13, wherein said antagonist is an antibody or functional fragment thereof.

15. The antagonist of claim 14, wherein said antibody, or functional fragment thereof, is selected from among a polyclonal antibody, a monoclonal antibody, a chimeric antibody, a humanized antibody, a human antibody, a labeled antibody, a Fab, a F(ab)₂, a F(ab′)₂, a scFv and a genetically engineered antibody.

16. The antagonist of claim 13, wherein said antagonist inhibits angiogenesis.

17. The antagonist of claim 13, wherein said antagonist prevents, inhibits, or treats an angiogenesis-dependent disorder, a cell proliferative disorder or a collagen-dependent disorder.

18. A composition comprising a pharmaceutically acceptable carrier or excipient and an antagonist of claim 13.

19. The composition of claim 18, further comprising a moiety selected from among a therapeutic moiety, an imaging moiety, a diagnostic moiety and a combination thereof.

20. The composition of claim 18, wherein the composition is substantially free of pyrogens.

21. A pharmaceutical package or kit comprising a composition of claim 18 and one or more packaging materials.

22. The pharmaceutical package or kit of claim 21, further comprising a label indicating use of the composition for inhibiting angiogenesis or an angiogenesis-dependent disease or disorder.

23. The pharmaceutical package or kit of claim 22, wherein the angiogenesis-dependent disease or disorder is selected from among ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and haemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

24. The pharmaceutical package or kit of claim 21, further comprising instructions for use.

25. An antagonist that preferentially binds to a binding site on a denatured collagen, wherein said binding site consists of an isolated polypeptide having an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

26. The antagonist of claim 25, wherein said antagonist is an antibody or functional fragment thereof.

27. The antagonist of claim 26, wherein said antibody, or functional fragment thereof, is selected from among a polyclonal antibody, a monoclonal antibody, a chimeric antibody, a humanized antibody, a human antibody, a labeled antibody, a Fab, a F(ab)₂, a F(ab′)₂, a scFv and a genetically engineered antibody.

28. The antagonist of claim 25, wherein said antagonist inhibits angiogenesis.

29. The antagonist of claim 25, wherein said antagonist prevents, inhibits, or treats an angiogenesis-dependent disorder, a cell proliferative disorder or a collagen-dependent disorder.

30. A composition comprising a pharmaceutically acceptable carrier/excipient and an antagonist of claim 25.

31. The composition of claim 30, further comprising a moiety selected from among a therapeutic moiety, an imaging moiety, a diagnostic moiety and a combination thereof.

32. The composition of claim 30, wherein the composition is substantially free of pyrogens.

33. A pharmaceutical package or kit comprising a composition of claim 30 and one or more packaging materials.

34. The pharmaceutical package or kit of claim 33, further comprising a label indicating use of the composition for inhibiting angiogenesis or an angiogenesis-dependent disease or disorder.

35. The pharmaceutical package or kit of claim 34, wherein the angiogenesis-dependent disease or disorder is selected from among ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and haemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

36. The pharmaceutical package or kit of claim 33, optionally, further comprising instructions for use.

37. An antibody, or functional fragment thereof, that preferentially binds to a binding site on a denatured collagen, wherein said binding site consists essentially of a polypeptide having an amino acid sequence set forth as SEQ ID NO: 81 (GPPGPP), wherein one or more proline residues is hydroxyproline.

38. The antibody, or functional fragment thereof, of claim 37, wherein said antibody, or functional fragment thereof, is selected from among a polyclonal antibody, a monoclonal antibody, a chimeric antibody, a humanized antibody, a human antibody, a labeled antibody, a Fab, a F(ab)₂, a F(ab′)₂, a scFv and a genetically engineered antibody.

39. The antibody, or functional fragment thereof, of claim 37, wherein said antibody, or functional fragment thereof inhibits angiogenesis.

40. The antibody, or functional fragment thereof of claim 37, wherein said antibody, or functional fragment thereof prevents, inhibits, or treats an angiogenesis-dependent disorder, a cell proliferative disorder or a collagen-dependent disorder.

41. A composition comprising a pharmaceutically acceptable carrier/excipient and an antibody, or functional fragment thereof, of claim 37.

42. The composition of claim 41, further comprising a moiety selected from among a therapeutic moiety, an imaging moiety, a diagnostic moiety and a combination thereof.

43. The composition of claim 41, wherein the composition is substantially free of pyrogens.

44. A pharmaceutical package or kit comprising a composition of claim 41 and one or more packaging materials.

45. The pharmaceutical package or kit of claim 44, further comprising a label indicating use of the composition for inhibiting angiogenesis or an angiogenesis-dependent disease or disorder.

46. The pharmaceutical package or kit of claim 45, wherein the angiogenesis-dependent disease or disorder is selected from among ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and haemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

47. The pharmaceutical package or kit of claim 44, further comprising instructions for use.

48. An antibody, or functional fragment thereof, that preferentially binds to an isolated polypeptide consisting essentially of an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

49. The antibody, or functional fragment thereof, of claim 48, wherein said antibody, or functional fragment thereof, is selected from among a polyclonal antibody, a monoclonal antibody, a chimeric antibody, a humanized antibody, a human antibody, a labeled antibody, a Fab, a F(ab)₂, a F(ab′)₂, a scFv and a genetically engineered antibody.

50. The antibody, or functional fragment thereof, of claim 48, wherein said antibody, or functional fragment thereof inhibits angiogenesis.

51. The antibody, or functional fragment thereof of claim 48, wherein said antibody, or functional fragment thereof prevents, inhibits, or treats an angiogenesis-dependent disorder, a cell proliferative disorder or a collagen-dependent disorder.

52. A composition comprising a pharmaceutically acceptable carrier/excipient and an antibody, or functional fragment thereof, of claim 48.

53. The composition of claim 52, further comprising a moiety selected from among a therapeutic moiety, an imaging moiety, a diagnostic moiety and a combination thereof.

54. The composition of claim 52, wherein the composition is substantially free of pyrogens.

55. A pharmaceutical package or kit comprising a composition of claim 52 and one or more packaging materials.

56. The pharmaceutical package or kit of claim 55, further comprising a label indicating use of the composition for inhibiting angiogenesis or an angiogenesis-dependent disease or disorder.

57. The pharmaceutical package or kit of claim 56, wherein the angiogenesis-dependent disease or disorder is selected from among ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and haemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

58. The pharmaceutical package or kit of claim 55, further comprising instructions for use.

59. An antibody, or functional fragment thereof, that preferentially binds to an isolated polypeptide consisting of an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

60. The antibody, or functional fragment thereof, of claim 59, wherein said antibody, or functional fragment thereof, is selected from among a polyclonal antibody, a monoclonal antibody, a chimeric antibody, a humanized antibody, a human antibody, a labeled antibody, a Fab, a F(ab)₂, a F(ab′)₂, a scFv and a genetically engineered antibody.

61. The antibody, or functional fragment thereof, of claim 59, wherein said antibody, or functional fragment thereof inhibits angiogenesis.

62. The antibody, or functional fragment thereof of claim 59, wherein said antibody, or functional fragment thereof prevents, inhibits, or treats an angiogenesis-dependent disorder, a cell proliferative disorder or a collagen-dependent disorder.

63. A composition comprising a pharmaceutically acceptable carrier/excipient and an antibody, or functional fragment thereof, of claim 59.

64. The composition of claim 63, further comprising a moiety selected from among a therapeutic moiety, an imaging moiety, a diagnostic moiety and a combination thereof.

65. The composition of claim 63, wherein the composition is substantially free of pyrogens.

66. A pharmaceutical package or kit comprising a composition of claim 63 and one or more packaging materials.

67. The pharmaceutical package or kit of claim 66, further comprising a label indicating use of the composition for inhibiting angiogenesis or an angiogenesis-dependent disease or disorder.

68. The pharmaceutical package or kit of claim 67, wherein the angiogenesis-dependent disease or disorder is selected from among ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; Sjogren's disease; acne rosacea; systemic lupus; retrolental fibroplasia; rubeosis; capillary proliferation in atherosclerotic plaques, and osteoporosis; cancer-associated disorders, e.g., solid tumors, tumor metastases, angiofibromas, Kaposi's sarcoma, benign tumors such as hemangiomas, acoustic neuromas, neurofibromas, as well as other tumors which require neovascularization to support tumor growth; hereditary diseases such as Osler-Weber Rendu disease and haemorrhagic teleangiectasia; plaque neovascularization; hemophiliac joints and wound granulation; fibrocystic diseases e.g., fibrosis and endometriosis, collagen based skin diseases e.g., psoriasis, scleroderma, eczema, platelet based disorders associated with collagen e.g., plaque formation, type II collagen arthritis, inflammatory diseases e.g., restenosis, diabetic retinopathy, rheumatoid arthritis, opthalmic uses e.g., macular degeneration and the like.

69. The pharmaceutical package or kit of claim 66, further comprising instructions for use.

70. A method of inducing an immune response in a patient, comprising administering to the patient the composition of any one of claims 6, 18, 30, 41, 52 and 63, wherein the composition comprises an anti-human antibody or fragment thereof that induces an effective host immune response against the binding site of said antibody or fragment thereof.

71. A method of blocking binding of a ligand to an extracellular matrix component comprising administering the composition of any one of claims 6, 18, 30, 41, 52 and 63, to a subject in need thereof.

72. A method of inhibiting angiogenesis or an angiogenesis-dependent disease or disorder in a subject comprising administering the composition of any one of claims 6, 18, 30, 41, 52 and 63, to a patient.

73. A method of preventing or treating a cancer or metastasis in a subject comprising administering the composition of any one of claims 6, 18, 30, 41, 52 and 63, to the subject.

74. A method for preventing or treating a cancer or a metastasis, comprising surgical removal of the cancer and concurrent administration of an anti-cancer agent and the composition of any one of claims 6, 18, 30, 41, 52 and 63, to a subject suffering from cancer.

75. A method of inhibiting angiogenesis or an angiogenic disease or disorder, comprising contacting a cell or tissue with a therapeutically effective amount of the composition of any one of claims 6, 18, 30, 41, 52 and 63.

76. A method, comprising contacting a cell with an antagonist or antibody or functional fragment thereof of any one of claims 1, 13, 25, 37, 48 and 59, wherein contacting inhibits binding of an integrin to an extracellular matrix component.

77. A method, comprising contacting a cell with a composition of any one of claims 6, 18, 30, 41, 52 and 63, wherein contacting inhibits binding of an integrin to an extracellular matrix component.

78. A method of preventing or treating a cell proliferative disorder, comprising administering to a subject having or at risk of having a cell proliferative disorder a therapeutically effective amount of the composition of any one of claims 6, 18, 30, 41, 52 and 63.

79. A method for treating diabetic retinopathy, macular degeneration or neovascular glaucoma in a patient comprising administering to the patient a therapeutically effective amount of the composition of any one of claims 6, 18, 30, 41, 52 and 63.

80. A method of monitoring the efficacy of the methods of any one of claims 70 to 79.

81. An antagonist that preferentially binds to a ligand of a denatured type IV collagen, wherein binding of the antagonist to the ligand blocks binding of the ligand to denatured type IV collagen, and said antagonist is a peptide.

82. The antagonist of claim 81, wherein said peptide consists essentially of an amino acid sequence set forth as SEQ ID NO: 81 (GPPGPP) wherein one or more proline residues is hydroxyproline.

83. The antagonist of claim 81, wherein said peptide consists essentially of an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

84. The antagonist of claim 81, wherein said peptide consists of an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

85. An isolated peptide consisting essentially of an amino acid sequence set forth as SEQ ID NO: 81 (GPPGPP) wherein one or more proline residues is hydroxyproline.

86. An isolated peptide consisting essentially of an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

87. An isolated peptide consisting of an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline.

88. A composition comprising a pharmaceutically acceptable carrier or excipient and an antagonist of any one of claims 81 to 84.

89. A composition comprising a pharmaceutically acceptable carrier or excipient and a peptide, or variant or peptidomimetic thereof, of any one of claims 85 to 87.

90. A vaccine comprising a composition selected from among one or more of:
a) an antagonist or a peptide of any one of claims 81 to 87;
b) an antagonist of claim 1, 13, or 25;
c) an antibody of claim 37, 48 or 59; and
d) an anti-human antibody (Ab1) that binds to a composition of claim 88 or 89.

91. A method of vaccinating a subject comprising administering the vaccine of claim 90.

92. A pharmaceutical package or kit comprising a composition of claim 88 or 89.

93. A pharmaceutical package or kit comprising a vaccine of claim 90.

94. A method for inducing a host immune response in a patient against an isolated peptide consisting essentially of, or consisting of, an amino acid sequence selected from among PGAKGLPGPPGPPGPY (SEQ ID NO: 2), PGAKGLPGPPGPPGPY (SEQ ID NO: 3), PGAKGLPGPPGPPGPY (SEQ ID NO: 4), PGAKGLPGPPGPPGPY (SEQ ID NO: 5), PGAKGLPGPPGPPGPY (SEQ ID NO: 6), PGAKGLPGPPGPPGPY (SEQ ID NO: 7), PGAKGLPGPPGPPGPY (SEQ ID NO: 8), PGAKGLPGPPGPPGPY (SEQ ID NO: 9), PGAKGLPGPPGPPGPY (SEQ ID NO: 10), PGAKGLPGPPGPPGPY (SEQ ID NO: 11), PGAKGLPGPPGPPGPY (SEQ ID NO: 12), PGAKGLPGPPGPPGPY (SEQ ID NO: 13), PGAKGLPGPPGPPGPY (SEQ ID NO: 14), PGAKGLPGPPGPPGPY (SEQ ID NO: 15), LPGPPGPPGPY (SEQ ID NO: 18), LPGPPGPPGPY (SEQ ID NO: 19), LPGPPGPPGP (SEQ ID NO: 20), LPGPPGPPG (SEQ ID NO: 21), LPGPPGPPG (SEQ ID NO: 22), LPGFPG (SEQ ID NO: 23), LPGPPG (SEQ ID NO: 24), LPGLPG (SEQ ID NO: 25), LPGSPG (SEQ ID NO: 26), LPGTPG (SEQ ID NO: 27), PGPPGPPGPY (SEQ ID NO: 28), PGPPGPPGP (SEQ ID NO: 29), GPPGPPGPY (SEQ ID NO: 30), GPPGPPGP (SEQ ID NO: 31), GPPGPPG (SEQ ID NO: 32), GPPGPPG (SEQ ID NO: 33), GPPG (SEQ ID NO: 34), GPPG (SEQ ID NO: 35) and FPGPPGPDGLPGSMGPPG (SEQ ID NO: 36) or a variant or peptidomimetic thereof, wherein P is hydroxyproline, comprising administering to the patient a vaccine composition of claim 65, wherein the composition comprises an anti-human antibody or fragment thereof that induces an effective host immune response against the binding site of said antibody or fragment thereof.

95. A method for inducing a host immune response in a patient against an isolated peptide consisting essentially of an amino acid sequence set forth as SEQ ID NO: 81 (GPPGPP) wherein one or more proline residues is hydroxyproline.

96. A method of blocking integrin binding to an ECM component comprising administering the composition of claim 88 or 89 to a subject.

97. A method of inhibiting angiogenesis or an angiogenesis-dependent disease or disorder in a subject comprising administering the composition of claim 88 or 89 to a patient.

98. A method of preventing or treating a cancer or metastasis in a subject comprising administering the composition of claim 88 or 89 to the subject.

99. A method for preventing or treating a cancer or a metastasis, comprising surgical removal of the cancer and concurrent administration of an anti-cancer agent or treatment and the composition of claim 88 or 89 to a subject.

100. A method of inhibiting angiogenesis, comprising contacting a cell or tissue with a therapeutically effective amount of an antagonist of claim 81 sufficient to inhibit angiogenesis.

101. A method of inhibiting angiogenesis, comprising contacting a cell or tissue with a therapeutically effective amount of a peptide of any one of claims 85 to 87 sufficient to inhibit angiogenesis.

102. A method, comprising contacting a cell with an antagonist of claim 81, wherein contacting inhibits binding of an integrin to an extracellular matrix component.

103. A method, comprising contacting a cell with a peptide of claim 88 or 89, wherein contacting inhibits binding of an integrin to an extracellular matrix component.

104. A method of treating a cell proliferative disorder, comprising administering to a subject having or at risk of having a cell proliferative disorder an amount of the composition of claim 88 or 89 effective to treat the cell proliferative disorder.

105. A method of monitoring the efficacy of the methods of any one of claims 91 and 94 to 104.

106. A method of imaging or diagnosing angiogenesis or an angiogenic-dependent disease or disorder comprising contacting the composition of any one of claims 6, 18, 30, 41, 52, 63, 88 and 89, with a sample wherein said composition comprises an imaging moiety or a diagnostic moiety.

107. A method for assessing proteomics profile of a sample, comprising:
a) dividing a plurality of antibodies into an unlabelled portion and a labeled portion;
b) attaching the unlabelled antibodies on a solid surface to form an array of unlabelled antibodies on said solid surface;
c) contacting said array of unlabelled antibodies formed in b) with a biosample to retain antigens contained in said biosample that specifically bind to said unlabelled antibodies; and
d) detecting said retained antigens by contacting said retained antigens with said labeled antibodies,
wherein proteomics profile of said biosample is assessed.

108. A method for assessing proteomics profile of a biosample, comprising:
a) dividing a plurality of peptides or peptidomimetics into an unlabeled portion and a labeled portion;
b) attaching the unlabelled peptides or peptidomimetics on a solid surface to form an array of unlabeled peptides or peptidomimetics on said solid surface;
c) contacting said array of unlabeled peptides or peptidomimetics formed in b) with a biosample to retain antigens contained in said biosample that specifically bind to said unlabeled peptides or peptidomimetics; and
d) detecting said retained antigens by contacting said retained antigens with said labeled peptides or peptidomimetics,
wherein proteomics profile of said biosample is assessed.

109. A method of selecting one or more cells comprising contacting a sample containing cells with an antagonist that preferentially binds to a binding site on a denatured collagen, wherein said binding site comprises an isolated peptide of any one of claims 85 to 87.
